SEQUENCE LISTING (50 sequences)

SEQ ID NO: 1 - DNA, 8497 bp, Streptococcus salivarius
gattctcaacattattgtcattgcttatggagcttgtacagggcaaggcgctgaatggttttatggtagcgccacaggtcttttatttgccttcacctacctttactcagctatcaatacgattttcgattttgatcaacgtttgtatgggtggtttagtttatttgtggcaattaatacgctaccagcagggattctttgcttaacatctggatacggtggtaatgcttggtatggtattatttggttcttgtggggtattctatggctaactgcctttattgaaattaaccttaagaagaacctaggaaaatttgtcccttacctagctatttttgaaggaattgtaacagcttggattccggggcttttgatgctttggggcaagtggtaagcattgatttaaggaggaaaaacgatgcaattgacaatgcgtgagcagaaaaaaatgatgattagccttgcggctatgattgctcaacgacgtaaagataaaggaatcaaattgaatcatccagaagcggttgctttgattacagactatgtgcttgaaggtgcaagagaaggtaaaacggttgcccaattgatggatgaagctcgcaatcttttaacacgtgaagatgttatggaaggtattgcggaaatgattccaatgattcaagttgaagctacttttacggacagtacaaaactggttactgttcatgatcctattcagtaaggagaaatgtaatgattccaggtgaataccatgtggcgagtgagccaattgattataacggtggttacgaagccattagtcttgaagtgaaaaatgtgggtgaccgtgctgctcaagtgggctctcactaccatttttatgaagcaaacgaatctggtcttcagtttgatcgtgaaaaggcgcgaggcaaacgtctagatattccagcaggtacagccattcgttttgagccaggtgaaacgaaaacagtacaacttattgactttggagggaaacgtcgtatttttggtttcaataacaaggtcaatggtttcttagactagaaagaggacaaatcgatgagttttaaaatggatcgtgaagagtatgctcaacactatggaccaactgtaggtgatagcgtacgtcttggagataccaatctttttgcagccattgaaaaagactttactgtttatggacaggaatctaagttcggtggcggtaaagttttgcgtgatggtatgggtgttagtgctacggaaacacgtgacaatccatcagttgttgataccattattacaggtgcaaccatcattgactatacaggtattattaaagcagatatcggtattcgtgatggtaagattgttgctatcggtcgcggtggtaacccagatacaatggacaatgtggactttgttgtgggtgctagtacagaagccattgctgctgaaggtttgattgtgactgctggtggtattgaccttcacgtgcactatatttctgccgaccttcctgaatttggtttggataacgggattactaccctctttggtggtggtactggtcctgctgatggaagtaatgcgacaacttgtacaccaggtaaattccatattactcgtatgttgcaagctgttgatgatatgcctgctaactttggtttccttgccaaaggtgttggttctgagactgaagtggttgaagagcaaattaaggccggtgcagcaggaattaaaacacacgaggactggggtgcgacttacgcaggtattgataattcccttaaagttgcggataaatacgatgtttcctttgcggttcacactgactctttgaatgagggtggatttatggaaaatactttggaatccttccaaggtcgtactgttcataccttccacaccgaaggttcaggtggtggacatgctccagatatcatggtttttgctggtaaggaaaatattttgccatcatcaactaacccaatcaacccatacaccacaaatgctattggtgagttgttagatatggttatggtttgccaccacttggatccaaaaattccagaagacgtctcttttgctgaatcacgtgtacgtaaacaaactgtagctgcagaagacgttcttcacgatatgggtgcccttagtatcatgacttcagatgccatggcaatgggacgtgtcggtgaagtggccatgcgttgttggcaactggctgataagatgaaggctcagcgtggtccacttgaaggggattcagagtttaacgataataaccgtatcaaacgttacgtggctaaatatacaattaaccctgccatcaccaatggtattgcagactatatcggttctgtagaagttggtaaatttgcagatttggttatctgggaaccagctcaatttggtgcaaaacctaagttggtgcttaagggtggtatgctaacttatggtgttatgggtgacgctggttcaagtcttccaacacctcaaccacgtatcatgcgtaaattatatggtgcttacggtcaagcggttcatgaaacaaatcttacatttgtttctcaatatgcttatgatcacggtatcaaagaagaaattggtttgaataagattgttcttcctgttaagaatacgcgtaacttgactaagcgtgatatgaagcttaatgactacgctccaaaaacaatccgtatcgatccacagacctttgatgtctttattgatgatgagttggttacttgtgaaccaatccatacgacatcattgtctcaacgttatttcttgttctaaggaagacgctatataaatgaggctggaatttttcctccaacctcttttgtattttatagccataacgtttttagtgctttattaagttgctatatgagtttgatgctagatttttaaaatgtaatagaaaaggaaaaagtatgatttttacaaaagtagatgctcttgttaaagatatctatgtggacaaataccatattgaaacagtcattctttcgagcgatgaccttaacaaaaaaattattcgtgtgaagagtgatcatgggaatgaatttggtattcgtcttgataagggacaaaaattgcaaaatggctctgccttttttatcgatgatcaccatgtcctagctattggtgttgagtcacaggatttgattgtcatttcacctaaagatatggatgaaatgggaatcacagctcacattcttgggaatactcataaaccgattgaggtgaaagacgccaagatttatttagaggttgatccagttgtagagcaagtcttgactcaaaaagagattgcctacacgattgaagaagtggtccttgataagcccctacgccatgtgaatttaactgcccatgaacattaatccctttgctaatgtgtctttgcaagattatcttgaaattgtgcaaattgtcgattcaacctttccaattggatcatttaaccactcttttgggatggaaaattatctgcgcgaagacactgtaacagatgataaaggttacgaggagtggcaagaagcctatttagctagtcagtttaaatatggtgaaggtcttgtaatcaaattggtttatgatgctatggctacagacaacttagagcaggtttggcattatgataaggtcttgacagtttcgacgcaagcgcgtgaaacaagacaggggactaaaatgattgctaaacaaatgcttcgacttattcaaaggctccatgctattccggtattggatgactatcagtccaaaatacgtaagggtgaggtcttcggcaatccagctattgtctttgcactctatgtgtttaacaagggcttgggatgtagtgaagctattgcactttatggctatagcgtgatttcgacgatggttcaaaatgctgttcgtgccattccacttggacagtttgctggacaagagattgttttacgtagcttttcacaattagaaaaaatgacacaagaaattcaagaactggatgcgtcctaccttggggccaatacgcctggtcttgaattagctcagatgaaacatgaaacacaggtattccgcctattcatgtcctaaaatatcaacaaggttgagaggaaaaaacaatgacaaaacgtactgtaattattggagttggtggacctgttggttcaggtaaaacccttttgcttgagcgtcttacacgacgtatgtccgacttaaatttagcagttattactaacgatatctatacaaaagaagatgctcttttcttggctaaaaattcaagcttagatgaagaccgtatcattggtgtagaaactggtggatgtcctcatactgctattcgtgaagatgcctctatgaactttgaagcgattgaaactcttcaagagcgctttaaccatgatttggatgttattttccttgaaagtggtggggataacttggctgcgaccttcagtcctgatttggttgatttcaccatttatattattgacgttgctcagggtgaaaaaatcccacgtaaggctggtcaagggatgattaagagtgatttgttcttgatcaataagactgaccttgctccttatgttggagccaacctagaccgtatgcgtgaagatacccttcatttccgtaacgaagattctttcattttcacaaatttgaataatgatgacaatgttaaggaagtggaagaatggattcgtaagaatttcctactagaggacttgtaagatgacacaagcatacgatggctttgtccatcttggattttcaaaccgaaatggtcgtacaatttcccacaagaaataccaagaaggcaactctcgagtatcggcggataattcagatgccaacggtgttccttactatttcctcattaatatgggtgggggatttgtcgagggtgagcagtatcaagtgaccattgatgttaataaagatgctcatgccttggtaacaacccaaacacctacctatgtttacaagtgtgagaaaggacagttgacacatcagaatacgtccatcacacttgaagaaaatagctatttggagtacatggctgatgaagtcattccctatttgagatcacgctatttccaaacaagtcgtattgatatggataagtctgcccacttgatttattcagatggtgtgacggcaggttggtctcatgaggatttgccgtttcaataccattattttcgtaatttgacacaaatctaccaagatgatgagcttgtttatagcgatcagaccctcttagagcctcagaaacaagatatgtttaaacttggttattttgaaggctggcgtaattataatagtttggtaatggtgtcaccaaatattgacgaggcttttgttaaggctttgcagaagcacttagaaaatctgaatttagagtctgattttgctatttcatccttagatatcccgggtctggtgttacgtatcttaggaaaaactgctgaggataatcgtcgcgtcatttattcttgtgcagactattttagacaagaaatacatggattaacccctttgaatttgagaaaaaatgatatgaggagataaaaaatgcatattcctgaaaattacttaagccctatgacttgtgcggcaatgggggcagttatgttgcctatttggtataaggctgtcaaggaagtgaaggtaaaggttgacactgataaaaaaacgattcctatgttgggaatcgggacttccttgtccttccttatcatgatgtttaatcttccagccccaggtggaacgagtgcccatgctgttggggcagtgctaattgctatattattaggaccttgggcctcctgtttagcagttagtgtggctctagctatgcaggctttgctatttggtgatggtgggattttggcctttggtgcgaatgccttttgtatggctgttgtcatgccatttgtgggttatgctgtttataaactcttgaataagtggacgaagaacaggataattgctagcttttttggaggttatattggaattgtagttgcggccctaactgttgcggttttactaggaattcaaccgattctctttaaagatagcagtggtaatccgctttacaatccataccctttgagagtgacgcttccagtaatgggcttgactcacctgcttatcggcttggtagaaggatttttcacagccggtgttcaagaattcattgaacgtttgaatattgataatactcaggaaataacgactaaaaaactacgtcctttattgctctttatcctagccttaattatcctaacgccacttggtttattggcgacgggaacagcttttgcagaatgggatgtcaaagagttggtagaaaaattgtctcattaccatgtggaagcccaagcgccaaaaggaatgttgaatggtttttcattcaatgccctcttcccagattatagtatcgcaggcattccagaagttttgggttatatcctgagcgctgcctctgctgttttgattttcttcatcctctatcgcttgattttcggtagaaaggttgaaaaatgattctgccagattggatgtcggaagagcgcccagtagtcactaaagtcggtagaaataactttcttatccggaatcgtcaccatctggaagctcttcttcaaaagtttgaaacgcatcctttaaaagtagcatcagtttttcatccaacagctaaggttttacttctctttttcttacttgtttcagtgggaattagccgaaatctcacagttttgtggattgtagccttgtttttaggagctggcgtggcttttttaccgcattctgttttagtaagaactttgaaaaaaactgtagtgttgttgatcttccctttagttctttatctaccgcatctcttacttagcggaggtcaatcgctctttctttttagacttcctttgattgctgtagccattgcttattattcagaaacgagtacaataagtgagatgttggcggcattaaaaggattgcattttcctgatcttgttctgctccagttagatatcaccataaaatatattgatgtccttggaaaacaattgatggatttgctcaaagggattgaagcgcgaagttttggtggcaatcatcgtttccggattggaagtaatatctggggaattttataccttaaagccatacgctatggtgaggaactgactcaagccatggaagcacgttgctttgttggtgagtatgtcaagtcatcacagtcattcacatggaaagactggctggccttgataagtctagtagcagtgattttaggacagattctgttaggaggatgagatgtttcaattgaatcaagtggcctgtgcctatgaacaaaaaaaggtctttactggtcttgatttggagattagacaaggacaatatgtgatgttgatgggggaaaatgggactgggaaatcaagtcttatcagtttattaactggcttcaagcaggaagaatctggacgtattcttttcttagggaaggacctcaaagaatggctgaaggacaaacgtcaaaaacgagatttttatagccgcctcggaatcctctttcaggatgtggatagtcaattatttaatagtactgtctatgatgagattgcttttggtcctcgtcagctaggtctgaccgaagaagaggtctcacagcgggtccaagacacactgtccctgcttaaaattgaagatttaagggatcgcgttccctatcaactgtctggtggagaaaagaagaaagtggcctttgccagtatcatggtaacgaatccagatgtgtatattcttgatgaacccttcaataatctttctaaagaatatgaagaattttttagggaacttctacatgaacttcattcagctgggaaaaccattattatgtctgctcatcacttcaagcaccttcatcatgaaaaggctgatgttcttctttttgaagatggcaaagctgatttcttttctgcccaggaagtgctcaataaccagcaagtgattgagcgtttgtcacattattaataactataagtaggaaatcgttattggttttctactttttctttgtcaaacaattaatacttttaggtgatagtattttcttatcaccttgattatgtttaagtattagttaagcctagtgaattctgttacaataaaaacaatcaatctcaaaggagagtattatgaaacttaaaaaaattcttggaattacaggtgtagctattgcttcagtagctttgcttgctgcatgttcatctaaatcatcaaaagaagcatctaaatcttcaggtgctaaagaaacaattaactttgccactgttgggacaacagcaccattctcatatgaagaaaatggtgaattgactggttacgatgtggaagtggccaaagcagtcttcaaagactctgacaaatatgaagtgaaattccaaaaaacagaatggtcttcagtcttcacaggtgatgactcagctaaatacca

SEQ ID NO: 2 - DNA, 9398 bp, Streptococcus gordonii
taattataggaaattatttgcctaaaaaagtacaagaaaacagacgccccctgaatagaaaactggcttttctttttgttggaataggatttttattgtttgtaattgctatcttctgtttgtaagcagaatacatagatatatctttatttgtaagcgtatttatgatatactgtcattatcaaatataaaagggttattatgattaataaggaacattatcggtttatccgccagcatcctgcatttgagaattttccagtagagtcttttgataaattagccattgaaattcaatttcataaggtttcgaaaggtcaaattatctttttttctggagatcggcgtgatcgcctttttcttctctatcaaggatatgctcgaatagaacagtatgatgctaccgatactttttcttatactgattatataaaaaaggggaatgttttcccttatggtggcatcttctttgatgagcgctatcattatactgcaagtgctgtgacacaggtggaatattttagtattcctatgaaattgtttgaagatttttctaagaaaaatgtgaatcagctgttgtttattacgcagaaactatccagaatcttggagtttcaagaattacgcttgcgaaatgttgttgcggtcagtgcaacagacagagttgttcagtccctttcgatcttatgtatggatttatgcaaaacaggggatgttttgccatttccaattagtatgaaggagttggctaaactaggagctacaacccgtgaaacagttaaccaggttctcaaaaggttgagagaagaaggccgtatcagctatgagcacaaacagttggtttttactgatagagaatattttatgaaatatttcaaagaaagttagtctactggaccgactttctttttttagcaaaaacaaagattttataaaaaaataaatattcgcaaaaatcataaaagatataaaatatgcaaagaaaacgcttcaaaacataaaaaaatttaaaaaaaatctaaaagtgataaaaatttggcatttagagtgtcagtttttttgtgtaagtgttttcaaaaaatgctagaataatatatgtaaacgggcttaggaaaacctaaaccgcaaagaacaaggaggaaagtagatgtctacacatccaattcatgttttctcagaaatcggaaaactgaaaaaagttatgttacatagacctggtaaagagttggaaaacttgatgccagactatctcgaacgtcttctctttgatgatattccgtttttggaagatgcacaaaaagaacacgacaactttgctcaagcgcttcgcaatgaaggtattgaagtgctatatctagaaaaactggctgctgagtctttgacctcaccagaaattcgcgaccaattcatcgaagaatatcttgatgaagcaaatatccgcggacgccaaactaaagtggctattcgtgagttgcttcaaggtattaaagataaccaagaattggttgaaaaaacaatggctggtgtacaaaaagctgaattaccagaaattccagaagcagcaaaaggcttgactgacttggtagaatcagactatccattcgctatcgatccaatgccaaacctttacttcacacgagatccatttgctacaattggtaacgcagtatcactcaaccacatgtatgcagatacacgtaaccgcgaaactttgtatggtaaatatatcttcaaataccatccagtttatggtggaaatgttgagcttgtttacaatcgtgaagaagatactcgtatcgaaggtggagatgagttggttctttctaaagatgtattggcagttggtatctcacaacgtactgatgctgcatcaattgaaaaattgttggtaaacatcttcaagaaaaacgttggcttcaagaaagtattggctttcgaatttgctaacaaccgtaaattcatgcacttggatacagtcttcacaatggtagactacgataaatttactattcacccagaaatccaaggcaatcttcgcgtcttctctgtaacttacgaaaacgaacaattgaagatcgttgaagaaaaaggtgatttggcagaacttcttgctgaaaaccttggtgttgaaaaagtaacattgattccatgtggagatggcaacgctgttgctgcagcacgcgaacaatggaacgatggttcaaacactcttacaatcgctccaggtgttgttgttgtgtatgaccgcaatacagttactaataagaaattagaagaatacggcttacgtttgattaagatccgcggaagtgaattggttcgcggtcgtggtggacctcgttgtatgtcaatgccattcgaacgtgaagaaatctaaacgttcaatatcttaagaaattctaatagatagaaagaggaaataaaagaatgacaaattcagtattccaaggacgtagcttccttgcagaaaaagactttacccgtgcagagttagaataccttattggtctttcagctcacttgaaagatttgaaaaaacgtaacattgagcaccgttaccttgctggtaaaaatatcgctctcttgtttgaaaaaacatctactcgtacgcgtgcagcctttactacagcagctatcgaccttggcgcacatccagaatatcttggtgctaatgatattcagcttggtaaaaaggaatcaactgaagatacagctaaagttttgggccgtatgtttgatggtattgaattccgtggtttcagccaacgtatggttgaagaattggcagaattctcaggtgttccagtttggaatggtttgactgacgaatggcacccaactcaaatgttagctgactacttgacagttcaagaaaactttggtcgcttagaaggcttgacattggtatactgtggtgacggacgtaacaacgttgctaatagcttactagtgactggtgcaattcttggtgttaatgttcacatcttctctccaaaagaactcttcccagaacaagaaattgttgaattggcagaaggatttgcgaaagaaagtggcgctcacatcttaatcactgaagatgctgacgaagctgtgaaaggtgctgatgtactttacactgacgtttgggtatcaatgggtgaagaagacaaatttgcagaacgtgttgctcttttgaaaccataccaagtaaacatggatttggttaagaaagctgataacgaagacttgatcttcttgcactgcttgcctgctttccacgacacaaatactgtttatggtaaggatgttgctgaaaaatttggcgtagaagaaatggaagtaactgacgaagtattccgcagtaaatatgctcgtcatttcgaccaagcagaaaaccgtatgcatacaattaaagcagttatggctgcaactttaggtaatctttacattccaaaagtataaccttaaaaacaattaaactgtaataccaacagctatgagggctgcgactaatagctttagtccgccctcatttttaatagtcaaacagtttgtctttctaaattgaaaaataaactggaggacattattgtaatcaaaattaaaacgcatttactttgcgttgaaggagaattatatggcaaatcgtaaaatcgttgtagccttgggaggaaatgccatcctttcatctgatccgtcagcaaaagcccagcaggaagccctagttgaaactgctaagcatttggtgaaactgattaaaaatggagatgaccttatcattactcacgggaatggtcctcaagtaggaaatctcttgttacaacatttagcagctgattctgaaaagaatcctgcttttccactcgactctctcgtagccatgacagaaggaagcattggctactggttgcaaaatgctttgcaaaacgctctcttggatgaaggaattgacaaaaacgttgcttcagttgtaacacaagtggttgtagataagaacgacccagcttttgttaacctcagcaaaccaatcggacctttctattcagaagaagaagcaaaagcagaagctgagaagagcggagcaactttcaaagaagacgctggacgtggctggcgtaaagtcgttgcttcaccaaaaccggttgacattaaggaaatcgacacaattcgtactcttttaaatgacggccaagtagttgtagctgctggtggtggcggtattcctgtcattaaggaagataacggtcatctttcaggagttgaagctgttatcgataaagactttgcatctcaacgtttggctgagttagttgaagctgacctcttcatcgtgttgacaggagtagactatgtctttgtaaattacaataaacctgatcaagaaaaattagaacatgttaatgttgctcagttggaagaatatatcaaacaagatcaatttgctccaggaagtatgcttccaaaagttgaagcagctattgctttcgttaacggtcgtccagagggtaaagcggttattacttcactagaaaatctgggtgctttgattgagtctgaaagcggaacaattattcaaaaagactgaaatcaattttgaactatagactagtttaaaagatttgctctaaaaaacactggtatttttcatgtcaatatgataaaatattggtgataaataaacattttcttggatatttattcaagaagagagcttggttttttgcactttgttagattttaggaggagaaaacaatgagtgaagaaacaaaaaaagggtttaggatgccttcttcttataccgtcttgattattatcattgctgttatggcagcactaacctggattattccggctggtcagtatgatgtcaacaaagaaggaaacctgattgctggaacatataaagaggttgcttctaaccctcaagggatttgggatgttctcatggcaccgattcgtgcgatgcttggacacgaacctacaaaggcagcgattgacgtttccttctttatcctgatggtaggtggtttccttggtgttgttaatgaaactggaaccttagatgtagggattgcttctatcgtgaagaagtacaaaggccgtgaaaaaatgttgattgtcatccttatgcctctgtttgcccttggtggtacaacttatggtatgggtgaagaaactatggctttctacccacttcttgttcctgttatgatggcggttggttttgatagtattacagccgtagccattatcttacttggatctcaaattggatgtttggcatccactctaaatccatttgcaacagttatcgcttcggatacagcaggcgtgccgacagcagatggtattgtgctacgtcttatcttctggtttgtaatggttgcaatgagcacttactttgtttatcgttatgcggataagattcaaaaagatcctaccaaatctttggtatatagccaaagagaagaagatttgaaacacttcaatgtaacggataacgatgatgcaccttctgtcttgagtaagaaacaaaaacatgttttatatctcttcattgcaacatttgttatcatggttgccagctttatcccttggacagaccttcatatcgatctttttgaaaactttaattcttggttaacaggtcttcctgtaattggtaaaattattggttcttcaactggggctttgggtacttggtacttcccagaaggcgcaatgctctttgcctttatgggtatcttgattggtatcgtttatggtcttaaagaagacaagattatctcagccttcatgaatggtgctgctgacttgctcagtgttgcccttatcgtagcgatcgcgcgtggtatccaagttatcatgaacgatggtatgattactgcgactatccttcactggggtgaacaaggacttaaaggtctgtcatctcaactattcattatcttgacttacattttctacttgccaatgtcattcttaatcccatcttcatctggtcttgccagtgcaacaatgggtatcatggcacctttaggagagtttgtcaatgtgaaaggaagcttgattatcactgcttaccaatcagcttcaggtgttcttaacttggtagcaccaacttcaggtatcgttatgggagctcttgctcttggccgtatcagccttggtacttggtggaaattcattggtaaattgattgtagccattatcgttgtcagcattttgttgcttctcttgggtaccttcattccagctattggttaagaaatgtgaggtgcttccatgaaaaattatctaacagaacaagtaaaaaaagaatttctcgaatctttgaaaactcttatttcctacccatctgttcttaatgaaaatgaaaatggaacaccttttggacaagctattcaagatgtcctagaaaaaactttagaaatttgtcgaggtataggttttacaacttatctcgatcctaaaggctattatggatatgcagaaatcggtcagggagaagaactcctggccgttctctgtcatttggatgttgttccatcaggtgaagaagcagattggcaaacaccaccgtttgtggcaactgaaaaagatggttatctctttggacgcggtgttcaggatgataaaggaccgtctatggcagctttgtatgctgttaaagcattgctggatagtggtgttcgctttaaaaaacgggtacgttttattttcggaacagatgaggaaacgctctggcgttgcatgggtagatacaatgaattagaagaaagggcgactcttggtttcgctcctgattcatcctttccattgacctatgctgaaaagggacttttacaggtcaagttgcatggaccagggtccgattttatcaaacttgaagctggggatgctttcaatgttgtaccagctaaagctagttaccaggggcctttcttagagaaagtaattgcgggcctaagagcaacaggctttgattacgaagtgtcagctaatcaggtgacggttcttggtgtttcaaaacatgctaaagatgctgctgagggagtcaatgcaatcgttcgactggctaaagtactccaagttcttgctccccatcctgctttagcttttattgctgaagcggtaggagaagatgcaacaggagcccacttatttggtcccgtttcagatgaaccatcaggaagtttatcttttaatattgccggattgacagtcagttccgaaaaatcagaaatccgaattgatattcgaattcctgttttagcagacaaagaaaaattagttcaaacattgaccgacaaggcatctgattatcgcttggtttatgaggagtttgattatctagcgcctttatatgtaccaaaagatagcgagttggtcagtactttgatgagtatttatcaagaaaaaactggtgacgatagtccagctatgtcatcgggtggagcaacctttgctcgtactatgccaaactgtgttgcctttggtgctcttttccctggtgctgagcagacggaacatcaggctaatgaacgagctaaactagatgatctttaccgggcaatggatatctatgcggaaacgatcttccgtttggctggagaataaaagaaaaggagttgaaaatctcaactccttttgcttatttactaaagaaaaatggtggggcaaattctttaagcttatcaaaacattcaatggcagacggattatcttcacaaatgatcaaacaaacatcatctccacagacagttgcaacaatttgaggtaattccagtgcatccagaatcgctccaaaagactgggctaggccgggtagagttttcatgaccacttgattctgaacgggccgtagcatgatgagggcgtcttccatataaaaacgcaggcgcttctcccagcgagaaggagcaatgctatttataacatagtaagaaatattgttttcagtgaccttgaccaggttgagggccttcatatctcgagataaggtcgattgggtaacaataacgccattggcttcaaggagttcctgaagttcttgttgagtatggacttttttctccataatcagggaacgaattaaacgatgtctactttctattttattcataaaattacctactatttgataaacatagcgtctccaaagctgaaaaagcggtaacgctcagcaatagcgtgttcgtaagcctttaaggttaggtctcgtccggcaaaggctgagactagcataactaaagttgatttaggtaagtggaaattggtcgaaaaggcatcaacaatttgccactggtagcccggtttgataaaaatattggtccaaccagaatcagcttggatatcgcctttgaacttatttcctatggtttctagagttcgtattgaagttgttccgacagcgataacacggtgaccatttgccttaacctcacggagggtagcggcagcttcttcagacaaagtgtaaaattcagaatgcatttcgtggtcatctagattgtcaacagaaacgggtcggaaggtgccaagtccgacgtgaagggtcaaataaaccaacttaactcccttggcttcaatttgtgcaagcagttctttagtaaaatgaagtccagctgttggagcagcagcggagccattctctttagcgtagacagtttggtagcgctcgcgatctgctaatttttcatgaatataaggaggcagaggcatttcacccaagctctctagcacttctaagaaaatcccttgatagtcaaagcggacaatccgtcccccgtgttctagttcatctacaaccgtagctgtcagtcgaccatccccaaaggaaacttgagcaccaactttcaaacgtttagctggttttgctaagacctcccattgatctccttcagtattctttaaaagtaagagttcaacgtgaccaccagttccaggtttctcaccgtagagacgagcaggcaggacccgagtgttattcatgacaagggcgtcgccaggctggagttgatcaataattgagtcaaaatgctggtcagaaaattctccgcttgatcggtctacaaccaataaacgagaggcatcgcgtttttcaagaggagtttgggcaataagctcctctggtaagtgaaagtcaaaatcggcagtgttcattatttctccttaaacagtccatataatccataaaaagtaagcacaacataaaattggaaagctagttagtaaaattccaatacaaagagctatcattcttaatgttttgatggctctaaaaaataggctgagtacgagaaaaacaacacccagtaaaaataaaagtaataaatacagtaacattcttatattatatcacgattttatctttctttgctggcaaaatttaaaatatggaaatttctatctt

SEQ ID NO: 3 - DNA, 8952 bp, Streptococcus rattus
  misc_feature (1336)..(1336): n is a, c, g, or t
  misc_feature (1353)..(1353): n is a, c, g, or t
  misc_feature (1392)..(1392): n is a, c, g, or t
  misc_feature (1450)..(1450): n is a, c, g, or t
  misc_feature (1698)..(1698): n is a, c, g, or t
  misc_feature (1734)..(1734): n is a, c, g, or t
cctgcccctcggagggtcatctaaagatgattaccaaatgttgcgaaatctagaggaatttcagtacttacctatagattgttttgaccaattaatggcagctacacacttaagaaaagcatctaaaaatcatgttttatttttgtaagggagataatcgggacaagttatttttaatcaaatctggctatgtaaaactagaagatacagatagctctggtacgtttttatatactgattatgtgaaacataatacaatattcccatatggaggtatgtttttagaaaaaacgtatgatttttcagcaaaagccattactgacattgaatatttttatattcctgttgatttgtatgagaactttgtggctaccaacagtacacaaatgagagttttatgtcaaaaattatcacgtttgttaaggattcatgaaatccgtttaaggaacgttgttacttcaagtgctcaggatcgaattgtacaatctttggcgatcctgctttttgatgtatgcgaggataatatcttacctttttctattactacggttgaaattgcagcattaagtgctacgacacgagaaacggtaagtcatgttttaaaatctttaaaagaaaagaacattgtagaattaagtggacgaaaattagtttttttaaacagggattactttttaaattatatttactgattggattgacggaaacagcgcaagaaaagggagtgggaaagaagccaaaaattgaaaattttggttctcaccgctccctaatttctaggcggtgacctgaaacggtctctccccagaccgtttcactcccacccccgcacagttgaatcggtcagctcaagagcgtaaaacgcgaacgaaactgccattcaacaatggttcattaggcactaaagtgcctaatgaactgtgcaggggaaactctaggttagctaaagctgacagagtttctcccaaccactgccttatgatgttaaaacgaactctaattgatgaggttggacttttttgcccaacctttttttaaaatattgatataaatgtctaagagtttttggctattattatcacctcttaaatattaaggaggctgggtctgctatttaacgattaccctgctttatactttttatagtaccaatatttttgtaaaacacttttcctgacaggaggtgatttgggcttcctatttttatttctccgacatctcataaacaggcttacagccaataatatcttatacggtctttttcacaaagtgagtattctgtctaaaaaagcatgtttaacgtatgggctatttcaattattactttgataaagcnaatgctatcaaatttgnttgctctacttgtgtcaaatgacagaaaagctgtaccgnttaatttttcgcataacaaaaggatgatcattacatagagcttagatgaactttttgntcaataagaactctgcggctgatttacaaagctgccttagttttaatagtctaaatgaatttataagttaaatattataaaaattttaattaatatttataaaatatacaggttaaagttaggttttattcgatgtaatcgctttcttttttagtttttataaattctttcatcaaaaaacgtgaaataaataacaattcaaatgcgaaaaaaatcttatttcatttataaaaaacctctwaaatggnaatttgtaataaatgagaaattaatgattctcaaantcgaaaggagtagttaacatgactcaaaaaagccctattcatgttttttctgaaattggaaaattgaagaaggtaatgttacatcgtcctggaaaagaaatcgaaaatcttatgccagattacttagatcgtcttttatttgatgatattccctttttagagaatgctcaaaaggagcatgatgctttcgctgatgcgcttcggcaagaaggtgtagaggttctttatttagaggaacttgcagctgagtcacttgtgaatgatgacattcgggaacaattcattgatgaatacttgtctgaagctaacattcgtggacgagcaactaaaaaagctatccgagaattactgctggaaattaaagataataaagaattaatcgaaaagacaatggccggtgttcaaaagtcggagattgctgacaggttaagtggtgaagaaaagggtctgactgacctagtggaatcagattatccttttgcgattgatccaatgccaaacttatatttcacacgtgatccttttgcagcaattggcaatggtgtatcattaaatcacatgttttctgaaacccgtaaccgtgaaaccctctatggtaaatacatttttacctatcacccagaatacggcggcaaagtgccattggtttataatcgttctgaatccactcgtattgaaggcggcgatgaactggtgctttctaaggatgttttggcagtcggtatttcacagcgtacggatgcagcttcaattgaaaaattattaattaatattttcaaagaaaatcttggatttaaaaaagtacttgcctttgagtttgctaataatcgtaaatttatgcatctagatacagtctttaccatggttgactatgataaatttactattcaccctgaaattgaaggagatcttcgtgtttactccgtaacatatgaagataataatttgcatattcaggaagaaaaaggagatcttgctgaactgctggctgaaaaccttggcgttgaaaaggttgaattgattcgctgcggtggaaataatttggttgctgcaggtcgcgaacaatggaatgatggttcaaataccttggctattgcaccaggtgttgtagttgtttataacagaaataccatcacaaatgccatccttgaatctaaaggtttgagattgataaaaattgagggtactgagctggttcgtggacgtggaggaccacgttgtatgtctatgccatttgaacgcgaatctatttaacagctttgcagcttatggtaatcttattatggatatatagcgtggcttgacaggctttgtactagaatggatttatcaagaactgatgaatctaatgattagcatttttataaaaacgaagcatagcagattgatcctgtttgcttaaaacaatagtaaaggaataatttaaaggagaaaaacatgactcaagtatttcagggacgcagtttcctagctgaaaaagattttacacgtgctgaattagaatatcttattgatttttcagctcatttgaaagatttgaaaaaaagaggggttccccatcattatttagaaggaaaaaatattgcacttttgtttgaaaaaacatctacgcggacacgctctgcttttacgactgcagccattgatttgggagctcaccctgaatacttaggggcgaatgatattcagttaggtaaaaaggaatcaactgaagatactgcaaaagtattgggccgtatgtttgatggtattgaatttcgtggttttagccaaagaaaggttgaagaacttgctgaattttctggtgtcccagtttggaatggcttgacagatgagtggcatccgacacaaatgttggcagattttcttactgttaaagagaattttggaaaattagaaggccttactctggtttactgtggcgatggtcgtaataatatggccaattcactcttggtaaccggtgctattcttggtgttaatgttcgtattttctcacctaaggaactcttcccagcagatgatatcgttaaattggctgaatcatatgctaaagaaagcggagctaaactgctgattacagaagatgcagatgaggctgttagaggagcagatgtcctttacacagacgtttgggtgtccatgggtgaagaatctaagtttgaagaacgtgttaaattattgaaaccttatcaggttaatatggaattaatcaaaaaagccggcaatgaaaaccttattttcctccactgcttaccagcatttcatgacactaacactgtttacggaaaagatatcgaagagaagtttggtgttaaagaaatggaagtgacagatgaggtcttccgcagttcttatgctcggcaatttgatcaagcagaaaaccgcatgcataccattaaagctgtgatggctgcaactctgggtaatttatttatccctaaagtataagtgataacagacagctaggagagctgagactaattttcttagttcagctcccctttttatttggtaataaaggaggcaaaatgacaaatcgtaaaatagtagttgcattaggaggtaatgccattttaacatcggatccatcggccgatgctcaaaaagctgctttagttcagacagctaaacatttagtgaaattaataaaaaatggcgataatttgattattactcatggtaatggtccgcaggttggtaatctgttgttacaaaatttggaagcgaactctgaaaaaaatcccgctcttcctcttgattctttagtggccatgactgaaggttctattggttactggcttcaaaatgctcttgaaaatgagctgattaaagaaggactggacaaggaagtcgcatctgtaataactcaagtcatcgttgataaaaacgatcctgctttcaaagacctaaccaagccgattgggcctttttatagcgaagaagaagctaaagaagaggctaagaaaaccggagcaacgtttaaagcggatgctggccgtggctggagaaaggtcgttgcttctccaaagccagtaagtatcaatgaattaggaacaattaagactctggtcaactctggaggaattgtcattgctagtggtgggggaggtatccctgttgttaaagaggataatggtacccttaaaggagttgaggcagtcatagacaaggactttgcttctgagaaattagcgacttcaattgaagcagatttatttatcattttaactggagtggactatgtttttgttaattacaataaaccaaatcaaaagaagctggaacatgtcactgttgcagagctagaagaatatatccaacaaaaccaatttgcgcctggttcaatgcttccaaaggttgaagcagcaatttcctttgtgaaaagtaggcccaatgctaaagctgtcattacatcgcttgagaatcttggagcattggtagaaaatgaaagtggcactattattgaaagtgttaaaggttaaaagaggagaacattgttatgtcagaaaaacctaaaaaaataggcttagtagccttaacagccttaattatcagttcatctataggttctgggatatttgcgattccaaccgacatggcatctgcagcggctcccggagcggctttaattgcttggttaatcgcaggtctgggtgttttagctttgtgcctgtctattgtcaatattggaagaaaaaagccagaactatctgggattgtcagttatgctgaggatggatttggtccattcagtggctttatcagtggctggggttactggctctcggcgtggttaggtaatgttgcctttgctactatgatgatgaaaacgttaggacgttttttcccaatttttggcgaaggcaataatatcgtttcaataaccgttgcatcagttatactgtggtgtatgtattacattgttaatagaggtgttgaaggggcagcttcactgaatactatcatcaccttatgtaagctcgttcctctcgcactatacattgtactggctatcttattctttgattttgataccttcatgaataatttttggggcactgcttctggaggctttgaatttgggaaaataatggagcaagttcaaaactcaatgatggtcattatgtgggtatttgtcggtgttgaaggggcagccatgatgtctgatcgagcccaaagcaagtctattattgggaaatcaacagttcttggtcttcttggtctcctagtcatatatgtgtctgcttccattttgccttatggtattatgacacaagaacaagttgcagcattacacagtccagcgatgggatatgttcttgtagataaagtgggaaactggttcccagttttagttaatattgcccttattatttctatatttggtagttggctatcttggacaatgttgcctgctgaaacaacattagttatggcgaatcgccacttgcttcctcaaaaatttggtgagttaaatgctgctggcgctcctactttttcacttgtatttatgacagggttaacacaaattttcatgtttactctacttttcacaaatcaagcttaccaatttgcctattcactatgtacagctgctatctttgtttcttggctttatgttaccttgtatcaaacaaaactgtctttcaaacttggcgaactgccacaaacccttgtaggattagttggttcaatcttttatttgtgggctatttgggcttctggcattgattatttccttctgtgtttgattgtctatcttttaggaattattctttatcgtcaagccagaaaggaaaaaggtattacagaaacattttcagctaaagagaaggttcttttagtattgattgtcgccggcgctgttattgcactttttagactctttacaggacagatttcaatttaataaaatcatatgtttagttgtcataactgaaaattggacttaagagagaataagaagtaaagttgatatatagatcgtgttacttgtcaatcagatgaggacgtttcgtaaaatcttctcaggacaggttatttcctgacaagtgctgtttttcacggaaataactgttacaataaaaatgggttgaattagagctagactaataaacagacttttgatgtttaaaaagacgagttgatttagatatggaaaggggtaaacagatgaatattcagccttttggtgttgaagaatggcttaatgtatgggaaaatgatgctatttatgatattgcaggcagttcaatttcttcaatgactttaaaagaaattctttccataggagataaacctcaagaggtcctaattgatgaacttttaaaaaagaaaatgaattatggctggattgagggttctcctgactttaaggaagaggtagctaaattatatgatcatgctaaacctaatcaaattttacaaacaaatggagcgactggagctaattttttagccttgtatgccctgattgaaaggggcgatcacattatttctttatatccgacttatcagcaactttatgatattcctagatcttttggagctgaagttgacttttgggagataaaagaagagaataattggctgccctcacttgatgacttgcagcatttaatcaagccaaatactaaaatgatttgtattaataatgccaataatccaacaggtgctgtcatggatcgctctttcttggaaaagcttgttgagctagcaagagcagctgatatttatatactatcagatgaagtttatcgtcctttagaagaggaacttgatgttccggctatttatgatctttacgataaaggaatttcaacgcacagcctatccaaaacctattcagttcctggtgtgcgcgtggggtggattgttgcaaatgatgagttatctgatttatttagaaaataccgcgattatacgatgatttgtgcaggtgtttttgatgactatatcgcgacgcatattcttaagaataaagacagagttatcgaaagaaataaaaaaatagttagcgagaatttacagattgtaaaagattgggttgccaaagaacctcgcgtatcactggttgttcccaaaaaagtatcaacttcatttattaaacttgatatccctgaggaaacagaaccattttgtatcaggttattaaaggaaaaaggtgttcttttggtacctggaaatagatttgatctacctggttatgctagactgggatattgtactgataaggcaactttaattaagggcttaagtgagttgtctgagtttctaagacaatttgattagctgttatctatgggtgtcacttgtcatatcaaatgtaataagttcactaatgaactagaattctgtgtttaggttgtttggcttactccaagcaaccttttggcattttgtttagtcgttatagagtctttaaactgttatttatttttaagcgatcatcataaataatggaactacattatttaattatgatataattgaattgactatataatatatattgggaatgtttatgaataaattattgcgccaaagtaagataaaaaaaataataaaactaaaatctataggtactcaagaagaattgaagcgtcagcttgaattggaaaaagtgtttgcaactcaggcaaccttatcccgagatatgcgagaactaggcctttttaaatcacgagataaagaaggacgtttgtattatgaaatacctgaaaatagtgtaagcatttttacaccagccatgctttattatattaagaaggtttctcactcagagtcattgctagttcttcatacaaatttaggagaggcagatgttttggctaatttgatcgatgaagccggtagttctgaaattttgggaacagtagctggtgctgatacgcttttagttatctgtcgtgataaagagacggctagccaactagaaaacgatgttctgtccagcttatgagttcatttgaaaaagctcttgaagctctgattgcgctgctgcgagaacacgacagtgtcatcgcttatcaagctgttgaaaaaaagattaagtctctgccggagctcagccatttagtttataaaatgaaagcctatcagcaggatgctgtgctttttcaaaagattgaaaaggcagaagctcaaaaagaagcagaccagcaggcggagaaactgggaaaaaatctagagtcgacccctcgaggggcagcaagc

SEQ ID NO: 4 - DNA, 5801 bp, Streptococcus mutans
ggaatatttccaatttacgggtgttctgcaaaatagtctttgtacaaggaaaagaattgttctcgtttattgatggccatcttgatataaatattgtgaatgtgagtttttactgtgccaacagagaggtaaagttccttagcaatctcttgattttgtttatgttccagcaaaagttggcaaatgtcttgttctcgttctgtcaagtggtaatagtcaaaaaagtcttgaatagcatctttagataaagcctttgtttgtaaagcttggtttcttttttccaagagaggataatcttttaaaaaataataaagaagaagccagcaggcaatgatggaaaagagatcttctgaaacatttcgattcataatttttgtattaaaaatactgtattgatcaacagtgaaaataacaaaagtgtcctcaattaaaatagcaacactggcaatcatagctagcaaagcgattaattttaaatattttttacctaacttgcttacattttccttcttactattgaaaaaggctatcaagcctgtataaaacagaagcaattgatttggtaggtaatagaggtatacctttgtagctgtattgggcagtgatggtagtgatggtaagagcataaagagaaaaatagctagtaagatgaggtattcccaccaaggcattttccgtttacttaaataatgaacaatccataactgacaaaagttgttaacgagaaaaacagttgttttaatgaaagaagttccttgaaaggaactgttgtaattggaagcaaaacttgaaataatttcagtcattgaaataacagaattatctaagataaaaaagagcagataaagagaagtgactaagtaaagtgtcttctttttttctcttataaagaagcttacagaaaaggccatggtaatggcataaagcatcattaataatagattatagatatagatgagttcaattttcatggaagaaccctcctttaatttctctttaatatctatagtaattatatcattcaaataaacttttattgaaaaggctttgtaaaaaaggcctaaaccttgggtttattttttaaaaacggtttatttcataaggtttagttagctaaacctttgtttgtaatcgtttacagctcaaagtttatagtaaactgccctttaaaaaaatatgatggaaatgaaaaaaatagaggaggcttctatgatgaaaaaaacagattatattacgacagaggatttttctaaagaagaattgctaaaattggtagatttatctttaaaaatcaaggcctgtatcaaaaatggctactatccccccttattggaacacaaaagtttagggatgatttttcaacaaacctcaacacgaacacgtgtttcctttgaaacagccatgagccaacttggaggtcatgcacaatatttagcaccgggacaaattcagcttggaggtcatgaaacgattgaggatacgtcaactgttctttcccgattggatgatatcttaatggcccgtgttgaacgtcaccaaagtgtagtagacttggctagatgtgcttctattccagttattaacgggatgtctgattataatcatccaactcaggaacttggagatctctgtacaatgatagaacatttgccagctggtaaaaagttagaagattgcaaagttgtctttgttggagatgcgacacaggtttgtttttctcttgctctaataacgactaaaatgggaatggaatttgttcactttggacctaaaggatttcaattaaacgacatgcataaggaaaagttagataaaatttgtgaacgatctggtggaaaatacactgtaactgataatgaagatgccattgaaggtgctgatttcctttatacagatgtttggtatggtctatacgaagcagaattatctgaggaagaacggatgcaaattttcttccctaaatatcaagtcgatagtcaaatgatggctaaagctggtgcggactgcaaattcatgcattgcttgccagcaactcgtggtgaagagattacagatgaagtgatggacggtcctcattctatttgctttgacgaagcagaaaatcgtttgacttccattagaggattgctcgtttatcttttaagggattatagggaaaagaatccttatgatttagtgaagcaggaaaaggctaaggaagaattagaaacttttttgaagccggaataggtaattatatgagaatgggacagactaccatgcttgatacgcaatcatgtctgtcctaaactcctaacttccctatttaaaagaaaggaagatacctatggaaggaaagaaaaaatttagtttatttagtgcagtactttctgttatttgtgtcgtctttgttgctgaagcggctgctccggtagctgctattgggaattctcaattcttttggtggctctttttattaattgcctttcttctaccttatggtttgatttcatctgaattgggaacaacttatattggtgatggtggtatctatgattgggtgaccaaggcttttggtcataaatggggctctcgagtggcttggtattattggattaattttccactctggctagcttctctggcagttatgacaccgggtttattaacaacagttactggacacaacttttcaactgttacagctattattgttgaactcatttttatttggctggttatttggattagtttttatcccgtgagtgatagtatttggattttaaatggtgcagctgtcattaaaatgttattggccttacttgttggtggcttgggcctttatgtggccctgaccaagggcatggcaaatgaaatgaccttaaagtcactgttgccttcttttaatctgaacagtctctcttatatttcagttattatttttaacctgctcggttttgaggttatttgtacttttgcaggagatatggaaaatcctaaaaagcaaattcctcaatctattattgttgcaggtctggtaattgcagctatctatattttttctgcttttggtattggcgtctcaattccaacggataagatttcaaccagcagtggtatgatggatagttttaaattattaacaggctcaacgggcggttggtttatcatgaccatggcttttctatttttattgaccttgtttggcaatatgatttcttggtctctcggtgttaataatacagcttcttatgctgcagaaaatggagacatgccccaattttttgctaaaagaagtcgcaaaagagatatgccaattggtgctgctcttgctaatggtattgttgctagcattgtggttgttattgccccatttttgcccaatcaagatttattctgggctttcttctccttaaacttagtcatgtttttattgtcttatgttcctgtatttccagcatttttcaagttgagaaaaatagatccggatacaccgcgtccttttaaggttagtggcaatgatagttttttgagattacttgttattttaccaatgattttaattatcatttccttgatttttactgctctaccactggcttttgattctgaaactttagcttcaaaattaccaataacaattggttctcttatttttatagggataggtgaacttattattatcatcaaaaaaataaagaaatgaggtaagaaaatggcaaaacgtattaaaaatacaactccaaaacaagatggctttagaatgccaggtgaatttgaaaaacaaaaacaaatttggatgctttggccttggcgcaatgataattggcggttgggagctaaacctgctcaaaaggcttttttagaagtagctgaggctattagtgagttcgagcctgtctctctttgtgttccgccactgcaatatgaaaatgctttggctcgcgtatcagaattgggtagtcataatattcgaattattgaaatgaccaatgatgatgcttggattcgtgactgtggtccaacatttctggtgaatgacaaaggagatttgcgtgcggttgattgggaattcaatgcctggggaggcttagtcgatggtctttattttccttgggaccaagatgctttagtagcacgtaaggtttgtgaaatagaaggtgtggattcttacaaaacgaaagattttgttcttgaaggaggttctatccatgtggatggcgaaggaaccgttttggtaacagaaatgtgtctgttacatcctagtcgtaatccgcatctgaccaaagaagatattgaagataaattgaaggactatcttaattgtgtaaaggttctttgggtcaaggatggcattgatccttatgaaacgaatggtcatattgatgatgttgcctgctttattcgtccgggggaagttgcctgcatctatacagatgataaggaacatcctttttatcaggaagctaaagcagcttatgacttcttgtctcaacagacagatgccaagggacgtcctttaaaggttcataaaatgtgcgtgaccaaggaaccctgttatctgcaggaagctgcaaccattgactatgttgaaggcagtattccacgtgaagaaggagaaatggcgattgcctcttatttgaatttcttgattgttaatggagggattattttaccgcagtatggggatgaaaatgatcaactagctaaacagcaggtacaggaaatgtttccagatagaaaagtcgttggtgtgagaacagaagaaattgcttacggtggtggcaatattcactgtattacacaacagcaacctgcaacttaaactaattaatcagtgaaaatggagaaaatgtatggcaaaaagaaaaattgtcattgcattagggggaaatgcaattttgtctagagatgcttctgccaaagcacagcaggcagcattggctcagactgccaaatatctggtccaattcattaaaaatggtgatgatttagtcattactcatgggaatggtccgcaggtaggtaatctgttattgcaacaaacagctgctgattcttatgacaatccagcgcttcccttggataccctagtggccatgacagaaggttccattagttattggttacaaaatgccttaatcaatgagttaaggaaacaatccattgataaggaagttgtgtctatggtaacagaagtacttgtatcagccgaagatcccgcttttgaccatcccagtaaacccatcggtccttttcttagtgaggaagaagcctatctgcaagaaaagatgactggtgctacttataaagtagatgcaggcagaggttggcgaaaagtagttgcttctcccaagccaattgccattcaggaaatagcaacgataaaatctttgcttaatacaggagctgttgttattacagcaggtggcggtggcattccggttattgaagaccctaaaacaaaagaattaatgggtgtggaagctgttattgataaagattttgccagtcaattattggccgaaaaaatcaaagctgatttatttattattttgactggtgttgatcatgtttatattcattatggtcaacctaatcaagaaaaattagaaaaagtaacagcaagtcagctaaaagcatggaaggatcaacaacaatttgcagcaggtagcatgctaccaaaagtagaagcagcaattgcctttgttgaagcacatcccagtggaaaagccattattacttctttagaaaatatagcaaatgttatttcagaaggaagtggcacacaaattacggctaattaagatgatgtttggaaa

SEQ ID NO: 5 - DNA, 20 bp, Streptococcus salivarius
gcttatcggcttggtagaag

SEQ ID NO: 6 - DNA, 17 bp, Streptococcus salivarius
gcttcgtgcttcaatcc

SEQ ID NO: 7 - DNA, 23 bp, Streptococcus salivarius
gttctgctccagttagatatcac

SEQ ID NO: 8 - DNA, 19 bp, Streptococcus salivarius
gcaatctcatcatagacag

SEQ ID NO: 9 - DNA, 19 bp, Streptococcus salivarius
ctcaaagaatggctgaagg

SEQ ID NO: 10 - DNA, 20 bp, Streptococcus salivarius
acctagaacgatttcctact

SEQ ID NO: 11 - DNA, 25 bp, Streptococcus salivarius
tagaaagaggacagatctatgagtt

SEQ ID NO: 12 - DNA, 26 bp, Streptococcus salivarius
ctcatttatataggtcgacccttaga

SEQ ID NO: 13 - DNA, 23 bp, Streptococcus salivarius
ctgaatttagagtctgattttgc

SEQ ID NO: 14 - DNA, 18 bp, Streptococcus salivarius
ggcattcgcaccaaaggc

SEQ ID NO: 15 - DNA, 20 bp, Streptococcus salivarius
gcttatcggcttggtagaag

SEQ ID NO: 16 - DNA, 22 bp, Streptococcus salivarius
ggctacaatccacaaaactgtg

SEQ ID NO: 17 - DNA, 23 bp, Streptococcus salivarius
gttctgctccagttagatatcac

SEQ ID NO: 18 - DNA, 19 bp, Streptococcus salivarius
gcaatctcatcatagacag

SEQ ID NO: 19 - DNA, 22 bp, Streptococcus gordonii
  misc_feature (3)..(3): n is a, c, g, or t
  misc_feature (10)..(10): n is a, c, g, or t
  misc_feature (14)..(14): n is a, c, g, or t
ggngatcgcnacgnaatcaatc

SEQ ID NO: 20 - DNA, 24 bp, Streptococcus gordonii
  misc_feature (6)..(6): n is a, c, g, or t
  misc_feature (18)..(18): n is a, c, g, or t
tgcatncgtagtttctcngctctg

SEQ ID NO: 21 - DNA, 27 bp, Streptococcus gordonii
caggtctatgtaacataacttttttca

SEQ ID NO: 22 - DNA, 13 bp, Streptococcus gordonii
  misc_feature (7)..(7): n is a, c, g, or t
ttgtgtntagaat

SEQ ID NO: 23 - DNA, 14 bp, Streptococcus gordonii
agaaaacgcttcaa

SEQ ID NO: 24 - DNA, 14 bp, Streptococcus gordonii
tgtaagtgttttca

SEQ ID NO: 25 - DNA, 18 bp, Streptococcus rattus
caagtatttcagggacgc

SEQ ID NO: 26 - DNA, 18 bp, Streptococcus rattus
catctgtcaagccattcc

SEQ ID NO: 27 - DNA, 22 bp, Streptococcus rattus
gacgatgtaacattaccttctt

SEQ ID NO: 28 - DNA, 22 bp, Streptococcus rattus
ctgtgtatgtctatgccatttg

SEQ ID NO: 29 - DNA, 20 bp, Streptococcus rattus
agctaggaaactgcgtccct

SEQ ID NO: 30 - DNA, 24 bp, Streptococcus rattus
tttagactctttacaggacagatt

SEQ ID NO: 31 - DNA, 24 bp, Streptococcus rattus
tgaatattcatctgtttacccctt

SEQ ID NO: 32 - DNA, 21 bp, Streptococcus rattus
agtgagttgtctgagtttcta

SEQ ID NO: 33 - DNA, 22 bp, Streptococcus rattus
tttatcttactttggcgcaata

SEQ ID NO: 34 - DNA, 27 bp, Staphylococcus aureus
ttgctctagagctctcaaatgacagaa

SEQ ID NO: 35 - DNA, 29 bp, Staphylococcus aureus
ttataaattcgagctccaaaaaacgtgaa

SEQ ID NO: 36 - DNA, 32 bp, Staphylococcus aureus
taaataacaattcgagctcgaaaaaaatctta

SEQ ID NO: 37 - DNA, 30 bp, Staphylococcus aureus
ttttgagtcatggatcctactcctttcgat

SEQ ID NO: 38 - DNA, 25 bp, Streptococcus mutans
cagattatatctagacagaggattt

SEQ ID NO: 39 - DNA, 29 bp, Streptococcus mutans
taccagctgggaatccttctatcattgta

SEQ ID NO: 40 - DNA, 25 bp, Streptococcus mutans
cagattatatctagacagaggattt

SEQ ID NO: 41 - DNA, 29 bp, Streptococcus mutans
taccagctgggaattcttctatcattgta

SEQ ID NO: 42 - DNA, 29 bp, Streptococcus mutans
tacaatgatagaagaattcccagctggta

SEQ ID NO: 43 - DNA, 26 bp, Streptococcus mutans
accgtccatgagctcatctgtaatct

SEQ ID NO: 44 - DNA, 25 bp, Streptococcus mutans
cagattatatctagacagaggattt

SEQ ID NO: 45 - DNA, 29 bp, Streptococcus mutans
taccagctgggaatccttctatcattgta

SEQ ID NO: 46 - protein, 293 aa, Streptococcus mutans
RVFCKIVFVQGKELFSFIDGHLDINIVNVSFYCANREVKFLSNLLILFMFQQKLANVLFSFCQVVIVKKVLNSIFRSLCLSLVSFFQERIIFKIIKKKPAGNDGKEIFNISIHNFCIKNTVLINSENNKSVLNNSNTGNHSQSDFIFFTLAYIFLLTIEKGYQACIKQKQLIWVIEVYLCSCIGQWWEHKEKNSDEVFPPRHFPFTIMNNPLTKVVNEKNSCFNERSSLKGTVVIGSKTNNFSHNNRIIDKKEQIKRSDVKCLLFFSYKEAYRKGHGNGIKHHIIDIDEFNFH

SEQ ID NO: 47 - protein, 350 aa, Streptococcus mutans
MMEMKKIEEASMMKKTDYITTEDFSKEELLKLVDLSLKIKACIKNGYYPPLLEHKSLGMIFQQTSTRTRVSFETAMSQLGGHAQYLAPGQIQLGGHETIEDTSTVLSRLDDILMARVERHQSVVDLARCASIPVINGMSDYNHPTQELGDLCTMIEHLPAGKKLEDCKVVFVGDATQVCFSLALITTKMGMEFVHFGPKGFQLNDMHKEKLDKICERSGGKYTVTDNEDAIEGADFLYTDVWYGLYEAELSEEERMQIFFPKYQVDSQMMAKAGADCKFMHCLPATRGEEITDEVMDGPHSICFDEAENRLTSIRGLLVYLLRDYREKNPYDLVKQEKAKEELETFLKPE

SEQ ID NO: 48 - protein, 452 aa, Streptococcus mutans
MEGKKKFSLFSAVLSVICVVFVAEAAAPVAAIGNSQFFWWLFLLIAFLLPYGLISSELGTTYIGDGGIYDWVTKAFGHKWGSRVAWYYWINFPLWLASLAVMTPGLLTTVTGHNFSTVTAIIVELIFIWLVIWISFYPVSDSIWILNGAAVIKMLLALLVGGLGLYVALTKGMANEMTLKSLLPSFNLNSLSYISVIIFNLLGFEVICTFAGDMENPKKQIPQSIIVAGLVIAAIYIFSAFGIGVSIPTDKISTSSGMMDSFKLLTGSTGGWFIMTMAFLFLLTLFGNMISWSLGVNNTASYAAENGDMPQFFAKRSRKRDMPIGAALANGIVASIVVVIAPFLPNQDLFWAFFSLNLVMFLLSYVPVFPAFFKLRKIDPDTPRPFKVSGNDSFLRLLVILPMILIIISLIFTALPLAFDSETLASKLPITIGSLIFIGIGELIIIIKKIKK

SEQ ID NO: 49 - protein, 369 aa, Streptococcus mutans
MAKRIKNTTPKQDGFRMPGEFEKQKQIWMLWPWRNDNWRLGAKPAQKAFLEVAEAISEFEPVSLCVPPLQYENALARVSELGSHNIRIIEMTNDDAWIRDCGPTFLVNDKGDLRAVDWEFNAWGGLVDGLYFPWDQDALVARKVCEIEGVDSYKTKDFVLEGGSIHVDGEGTVLVTEMCLLHPSRNPHLTKEDIEDKLKDYLNCVKVLWVKDGIDPYETNGHIDDVACFIRPGEVACIYTDDKEHPFYQEAKAAYDFLSQQTDAKGRPLKVHKMCVTKEPCYLQEAATIDYVEGSIPREEGEMAIASYLNFLIVNGGIILPQYGDENDQLAKQQVQEMFPDRKVVGVRTEEIAYGGGNIHCITQQQPAT

SEQ ID NO: 50 - protein, 316 aa, Streptococcus mutans
MAKRKIVIALGGNAILSRDASAKAQQAALAQTAKYLVQFIKNGDDLVITHGNGPQVGNLLLQQTAADSYDNPALPLDTLVAMTEGSISYWLQNALINELRKQSIDKEVVSMVTEVLVSAEDPAFDHPSKPIGPFLSEEEAYLQEKMTGATYKVDAGRGWRKVVASPKPIAIQEIATIKSLLNTGAVVITAGGGGIPVIEDPKTKELMGVEAVIDKDFASQLLAEKIKADLFIILTGVDHVYIHYGQPNQEKLEKVTASQLKAWKDQQQFAAGSMLPKVEAAIAFVEAHPSGKAIITSLENIANVISEGSGTQITAN

(19) United States
(12) Patent Application Publication
(10) Pub. No.: US 2007/0212311 A1
(43) Pub. Date: Sep. 13, 2007

(54) Recombinant Alkalinizing Bacteria

(75) Inventors: Robert A. Burne, Gainesville, FL (US); Yi-Ywan Margaret Chen, Gainesville, FL (US)

Correspondence Address: AKERMAN SENTERFITT, P.O. BOX 3188, WEST PALM BEACH, FL, 33402-3188, US

(73) Assignee: UNIVERSITY OF FLORIDA RESEARCH FOUNDATION, INC., GAINESVILLE, FL (US)

(21) Appl. No.: 10/574,730

(22) PCT Filed: Oct. 7, 2004
(86) PCT No.: PCT/US04/33214

§ 371 Date: Jan. 5, 2007

Related U.S. Application Data
(60) Provisional application No. 60/509,175, filed on Oct. 7, 2003.

(51) Int. Cl. A61K 8/66; C07H 21/00; C12N 1/21

(52) U.S. Cl. 424/50; 435/252.35; 536/22.1

(57) ABSTRACT

Compositions and methods utilizing recombinant alkalinizing bacteria which include isolated nucleic acid constructs. The genetically engineered cells express at least one alkali producing enzyme, such as a urease, an arginine deiminase or an agmatine deiminase, which produce the alkali ammonia. When used in the mouth of a subject, the presence of alkalis raises the pH, reducing the incidence of cavities.

### FIELD OF THE INVENTION

The invention relates generally to the fields of microbiology, molecular biology and genetics. More particularly, the invention relates to recombinant bacteria engineered to express enzymes that produce alkalis such as ammonia.

### BACKGROUND

Dental caries (cavities) is one of the most common infectious diseases of humans. Caries result from changes in the balance of bacteria flora that colonize the teeth in the form of plaque. Because of periodic ingestion of dietary carbohydrates, biofilms colonizing the tissues of the mouth are continually subjected to fluctuations in environmental conditions. Environmental factors shown to have the most profound influence on the pathogenic potential of oral biofilms are pH and the source and availability of nutrients.

Dental biofilms undergo repeated cycles of demineralization of the tooth enamel due to acids produced from bacterial glycolysis. In the healthy state, the enamel-destroying demineralization phases are followed by periods of enamel-promoting alkalinization. Alkalinization can result from several factors, including diffusion of acids away from the biofilms, buffering by salivary components such as bicarbonate and peptides, and by bacterial metabolism resulting in products such as ammonia that raise the extracellular pH. Dental caries occurs when the acidification phases outweigh the alkalization phases, allowing for establishment of a more acidogenic, less alkalinogenic flora. Lower plaque pH values are associated with enhanced and prolonged enamel demineralization.

### SUMMARY

The invention relates to genetically engineered bacteria that promote alkalinization of dental biofilms. The bacteria comprise nucleic acid constructs having components necessary to express functional alkalinizing (alkali-producing) enzymes, such as the ammonia-producing enzymes urease, arginine deiminase and agmatine deiminase. Following introduction into the oral cavity and colonization of the plaque, bacteria according to the invention produce alkalis such as ammonia from urea, arginine, or agmatine.

In a preferred embodiment, the invention provides a recombinant bacterial cell comprising an isolated nucleic acid construct. Preferably, the recombinant cell expresses at least one alkalinizing enzyme and the cell is a bacterial strain that colonizes dental plaque. Preferably, the bacterial strain is at least one selected from the group consisting of Streptococcus mutans, Streptococcus sanguinis, Streptococcus gordonii, Streptococcus parasanguis, Streptococcus vestibularis, Streptococcus oralis, and Streptococcus mitis.

In another preferred embodiment, the alkalinizing enzyme is an ammonia-producing enzyme. Preferred ammonia-producing enzymes include but are not limited to urease, arginine deiminase, agmatine deiminase, and the like.

In another preferred embodiment, the nucleic acid construct comprises at least one gene cluster encoding a urease and/or a nickel transporter. Preferably, the urease is encoded by a construct comprising a gene cluster, such as ureIABCEFGDMQO, variants or fragments thereof.

In another preferred embodiment, the construct comprises at least one gene cluster encoding an arginine deiminase system. Preferably, the arginine deiminase system is encoded by a construct comprising a gene cluster, such as arcABCDTR, variants or fragments thereof.

In another preferred embodiment, the nucleic acid construct comprises at least one gene cluster encoding an agmatine deiminase system. Preferably, the agmatine deiminase system is encoded by a construct comprising a gene cluster, such as aguBDAC and a transcriptional regulator located upstream of the agu gene cluster, variants or fragments thereof.

In one embodiment, a preferred method for reducing acidification of dental plaque in a subject is provided. Preferably, the method comprises the steps of (1) providing a subject having at least one tooth with dental plaque disposed thereon, the tooth being in a fluid environment and the fluid environment at a first pH; (2) contacting said tooth with a composition comprising at least one alkalinizing recombinant bacterial cell; (3) the recombinant bacterial cell colonizes the dental plaque and producing at least one alkali from chemicals in the fluid environment; (4) the alkali raises the pH of the fluid environment to a second pH, wherein the second pH is higher than the first pH.

Preferably, the production of the alkali base by the recombinant bacteria occurs in the absence of exogenous nickel. In accordance with the invention, a alkali includes, but not limited to ammonia. The subject is preferably a mammal, wherein the human is preferred.

In another embodiment, a preferred composition comprises at least one recombinant bacterial cell including an isolated nucleic acid construct, said cell expressing at least one alkalinizing enzyme, in a carrier. Preferably, the nucleic acid construct comprises at least one gene cluster encoding a urease enzyme.

In another preferred embodiment, the nucleic acid construct further comprises a nickel transporter.

In other preferred embodiments, the nucleic acid construct comprises at least one gene cluster encoding an arginine deiminase system and/or an agmatine deiminase system. The recombinant bacterial cell comprising the nucleic acid construct can comprise any one, or combination thereof, of gene clusters.

In another preferred embodiment, the composition further comprises a carrier which can be a pharmaceutical composition, a chewing gum, a toothpaste, a lozenge, a powder, a gel, an ointment, a cream, a liquid, a mouthwash, a rinse, and a candy.

In another preferred embodiment, an isolated nucleic acid is provided, comprising the nucleotide sequence of any one of SEQ ID NO:1, SEQ ID NO: 2, SEQ ID NO: 3 and SEQ ID NO: 4, or combinations thereof. In accordance with the invention, nucleic acid sequences comprise isolated nucleic acid sequences that are about 50% homologous to SEQ ID NO: 1; preferably, the isolated nucleic acid sequences are about 75% homologous to SEQ ID NO: 1; more preferably, the isolated nucleic acid sequences are about 85% homologous to SEQ ID NO: 1; more preferably, the isolated nucleic acid sequences are about 90%, homologous to SEQ ID NO: 1; more preferably, the nucleic acid sequences are 95%, 96%, 97%, 98%, 99%, 99.9% homologous to SEQ ID NO: 1.

In another preferred embodiment, nucleic acid sequences comprise isolated nucleic acid sequences that are about 50% homologous to SEQ ID NO: 2; preferably, the isolated nucleic acid sequences are about 75% homologous to SEQ ID NO: 2; more preferably, the isolated nucleic acid sequences are about 85% homologous to SEQ ID NO: 2; more preferably, the isolated nucleic acid sequences are about 90% homologous to SEQ ID NO: 2; more preferably, the isolated nucleic acid sequences are 95%, 96%, 97%, 98%, 99%, and 99.9% homologous to SEQ ID NO: 2.

In another preferred embodiment, isolated nucleic acid sequences comprise nucleic acid sequences that are about 50% homologous to SEQ ID NO: 3; preferably, the isolated nucleic acid sequences are about 75% homologous to SEQ ID NO: 3; more preferably, isolated nucleic acid sequences are about 85% homologous to SEQ ID NO: 3; more preferably, isolated nucleic acid sequences are about 90% homologous to SEQ ID NO: 3; more preferably, isolated nucleic acid sequences are 95%, 96%, 97%, 98%, 99%, and 99.9% homologous to SEQ ID NO: 3.

In another preferred embodiment, isolated nucleic sequences comprise nucleic acid sequences that are about 50% homologous to SEQ ID NO: 4; preferably, isolated nucleic acid sequences are about 75% homologous to SEQ ID NO: 4; more preferably, isolated nucleic acid sequences are about 85% homologous to SEQ ID NO: 4; more preferably, isolated nucleic acid sequences are about 90% homologous to SEQ ID NO: 4; more preferably, isolated nucleic acid sequences are 95%, 96%, 97%, 98%, 99% and 99.9% homologous to SEQ ID NO: 4.

In another preferred embodiment, the nucleic acid construct comprises a pMJB8 vector, pMC340A vector, a pMC340B vector, a pMC341A vector, a pMC341B vector and/or a pMC321 vector.

In another preferred embodiment, the expression of the alkali producing genes are independent of pH. The compositions disclosed herein, retain the capacity to break down urea at pH 3 and urea can be metabolized from about pH 3 (or a little below) up to pH 10.

In other preferred embodiments, the compositions of the invention can be used to treat disease which lower pH such as vaginal infections, urinary tract infections and the like.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991, and Lewin, Genes VII, Oxford University Press: New York, 1999. Commonly understood definitions of microbiology can be found in Singleton and Sainsbury, Dictionary of Microbiology and Molecular Biology, 3rd edition, John Wiley & Sons: New York, 2002.

Although methods and materials similar or equivalent to those described herein can be used in the practice or testing of the present invention, suitable methods and materials are described below. The particular embodiments discussed below are illustrative only and not intended to be limiting.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic diagram of the ure operon of Streptococcus salivarius 57.I, according to an embodiment of the invention.

FIG. 2 is three graphs showing urease specific activities at pH 7, 6 and 5 in S. salivarius 57.I (wild type) and a UreMQO-deficient strain grown in media at pH 5, 6, and 7 comprising varying concentrations of NiCl₂, according to an embodiment of the invention. The specific activities are expressed as nmol urea hydrolyzed min⁻¹ (mg of total protein)⁻¹. Values shown are averages from multiple reaction sets from three independent samples. All values fell within the linear range of the standard curve. All reactions were performed in triplicate.

FIG. 3 is a schematic diagram showing the gene order and arrangement of the arc operon of S. gordonii DL1, according to an embodiment of the invention. The molecular mass in kDa and the pI of each gene product are shown.

FIG. 4 is a graph showing AD specific activity in wild-type S. gordonii DL1 and in an ArcR-deficient derivative, according to an embodiment of the invention. Cells were grown under growth conditions of 10 mM glucose or galactose, with or without additional 1% arginine.

FIG. 5 is a graph showing the composition of biofilms formed in the presence of urea, according to an embodiment of the invention. Biofilms were cultivated in complete BMMUG medium for the entire 11 day period. Organisms were recovered from the constant depth film fermenter (CDFF) and enumerated by viable counting on selective media. Viable counts are expressed as the average of the log CFU obtained per pocket, which provides the number of organisms that could be recovered from each of the five recessed pockets in the sample pans. Error bars depict standard deviation. The data represent at least two complete runs of 11 days. All platings were performed in triplicate using three separate sample pans.

FIG. 6 is a graph showing composition of biofilms before and after removal of urea from the medium, according to an embodiment of the invention. Biofilms were cultivated in complete BMMUG medium for 7 days and then in BMMG for days 8 through 11. Organisms were recovered from the CDFF and enumerated as described for FIG. 5.

FIG. 7 is a graph showing composition of biofilms formed with a urease-deficient Streptococcus salivarius, according to an embodiment of the invention Biofilms were cultivated in complete BMMUG medium for 7 days and then in BMMG for days 8 through 11, then organisms were recovered and enumerated.

FIG. 8 is a graph showing composition of biofilms formed with a urease-deficient Actinomyces naeslundii, according to an embodiment of the invention. Biofilms were cultivated in complete BMMUG medium for 7 days and then in BMMG for days 8 through 11, followed by recovery and enumeration of organisms.

FIG. 9 is a schematic diagram of a gtfG-based integration vector, according to an embodiment of the invention.

FIG. 10 is a schematic diagram of a mannitol-based integration vector, according to an embodiment of the invention.

FIG. 11 is a schematic illustration of the reconstructed urease gene cluster from S. salivarius 57.I.

FIGS. 12A to 12D are schematic illustrations of novel cloning vectors for introduction of foreign DNA into the mannitol locus of Streptococcus mutans and each panel shows the different restriction enzyme sites. (UA159#, intergenic region between Streptococcus mutans UA159 glmS and mtlA1. UA159*, intergenic region between mtlD and phnA.

FIG. 13 is a schematic illustration of a cloning vector for introduction of foreign DNA into the mannitol locus of Streptococcus mutans. The cat gene was amplified from pC194 with a BamHI site immediately 5′ to the ATG and a SphI site 3′ to the terminator. This PCR product (0.76 kbp) was subsequently cloned into pGEM-3Zf(+) at BamHI-SphI site to generate pMC286.

### DETAILED DESCRIPTION

The invention recombinant cells expressing alkali producing enzymes, such as urease, arginine deiminase, and/or the agmatine deiminase system, which produce ammonia. In particular, these recombinant cells are used for the treatment of dental biofilms, plaque and in other medical conditions which result in the alteration of the normal pH environment.

In preferred embodiments, recombinant cells comprising nucleic acid constructs encoding genes necessary for urease, arginine deiminase or agmatine deiminase expression are described. Host cells comprising such constructs or expression vectors include, but not limited to strains of Streptococcus mutans (S. mutans) or other strains of dental plaque bacteria, such as S. gordonii, S. sanguinis, S. parasanguis, S. vestibularis, S. oralis or S. mitis. Preferred cariogenic or periodonto-pathogenic bacteria may be from the genera Actinobacillus, Actinomyces, Bacteroides, Capnocytophaga, Eikenella, Eubacterium, Fusobacterium, Haemophilus, Lactobacillus, Peptostreptococcus, Porphyromonas, Prevotella, Rothia, Selenomonas, Streptococcus, Treponema, Wolinella.

In a particularly preferred embodiment, a recombinant host cell is exemplified by S. mutans. As described in the examples which follow, recombinant S. mutans bacterial cells express a fully functional urease enzyme which increases extracellular pH without the need for addition of exogenous nickel ions. The nucleic acid expression vector in this strain includes genes encoding a high affinity Ni²⁺ transporter identified from ureolytic streptococci. In other examples, recombinant strains of Streptococcus mutans or other strains of dental plaque bacteria express the genes encoding arginine deiminase and agmatine deiminase.

Dental plaque is a soft material formed of a complex mass of the bacteria that adheres to the very thin pellicle layer, formed primarily of salivary proteins, which surround each tooth. A 5-day-old plaque, if not disrupted as by brushing, can reach a thickness of about 60 μm. A cariogenic plaque containing a high proportion of S. mutans, can often contain 2×10⁸ bacteria per mg wet weight and can rapidly ferment sucrose, glucose, or fructose to generate enough acid to lower the pH of the plaque to 5.5 or lower; whereupon demineralization of surface enamel occurs.

The dental plaque in human oral cavity consists mainly of glucan, a composite of carbohydrates. Glucan is either dextran, water-soluble, or mutan, water-insoluble. Glucan is synthesized from sucrose by the glucosyltransferase secreted from Streptococcus mutans. Mutan has α-1,3 linkages so that it is insoluble in water, and makes the main matrix of the dental plaque. Generally, dental plaque, adherent to the surface of teeth, provides a suitable habitat at which Streptococcus mutans as well as other bacteria proliferate, and cause dental caries because food residues cling to it. Therefore, it is an object of the present invention to provide novel bacteria which moderate the pH drop in plaque from carbohydrates, allow plaque to return to neutral pH values faster after an acid challenge, and to prevent sustained acidification of dental plaque. Enamel begins to be damaged around pH 5.5.

In nutrient limited ecosystems, bacteria have a marked tendency to adhere to surfaces and initiate the formation of a biofilm. A biofilm is a community of microbes, embedded in an organic polymer matrix, adhering to a surface. The biofilm matrix is a collection of microcolonies with water channels in between and an assortment of cells and extracellular polymers (polysaccharides, glycoproteins, proteins). Bacterial extracellular polysaccharides are composed of homo- and heteropolysaccharides of particularly glucose, fucose, mannose, galactose, fructose, pyruvate, mannuronic acid or glucuronic acid based complexes. The different bonds between the saccharides give rise to a multitude of different polysaccharides including levans, polymannans, dextrans, cellulose, amylopectin, glycogen and alginate. Bacteria growing in biofilms are more resistant to antibiotics and disinfectants than bacteria growing in other environments and the resistance increases with the age of the biofilm. Bacterial biofilm also exhibits increased physical resistance towards desiccation, extreme temperatures or light.

The below described preferred embodiments illustrate adaptations of these compositions and methods. However, from the description of these embodiments, those having ordinary skill in the art will recognize that other aspects of the invention can be made and/or practiced based on the description provided below.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Definitions

Prior to setting forth the invention the following definitions are provided:

As used herein, the terms “exon” and “intron” are art-understood terms referring to various portions of genomic gene sequences. “Exons” are those portions of a genomic gene sequence that encode protein. “Introns” are sequences of nucleotides found between exons in genomic gene sequences.

“Amplification” relates to the production of additional copies of a nucleic acid sequence.

As used herein, the singular forms “a”, “an” and “the” include plural referents unless the context clearly dictates otherwise.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

A “vector” is a replicon, such as plasmid, phage or cosmid, to which another DNA segment may be attached so as to bring about the replication of the attached segment.

A “replicon” is any genetic element (e.g., plasmid, chromosome, virus) that functions as an autonomous unit of DNA replication in vivo, i.e., capable of replication under its own control.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. Preferably, the heterologous DNA includes a gene foreign to the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non transcribed strand of DNA (i.e., the strand having a sequence homologous to the mRNA). A “recombinant DNA molecule” is a DNA molecule that has undergone a molecular biological manipulation. The terms “nucleic acid molecule” or “polynucleotide” will be used interchangeably throughout the specification, unless otherwise specified.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

As used herein, the term “oligonucleotide specific for” refers to an oligonucleotide having a sequence (i) capable of forming a stable complex with a portion of the targeted gene, e.g. by either strand invasion or triplex formation, or (ii) capable of forming a stable duplex with a portion of a mRNA transcript of the targeted gene a mechanism also called antisense.

As used herein, the terms “oligonucleotide” or “primers” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorthiorate, methylphosphonate, and the like. Oligonucleotides are capable of specifically binding to a target polynucleotide by way of a regular pattern of monomer-to-monomer interactions, such as Watson-Crick type of base pairing, Hoögsteen or reverse Hoogsteen types of base pairing, or the like.

The oligonucleotide may be composed of a single region or may be composed of several regions. For example, hinge regions comprising different lengths and base composition. The oligonucleotide may be “chimeric”, that is, composed of different regions. In the context of this invention “chimeric” compounds are oligonucleotides, which comprise two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties.

Chimeric oligonucleotides can be formed as mixed structures of two or more oligonucleotides, modified oligonucleotides, oligonucleosides and/or oligonucleotide analogs as described infra. The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N₆-methyladenine, 7-deazaxanthine, 7-deazaguanine, N₄,N₄-ethanocytosin, N₆,N₆-ethano-2,6-diaminopurine, 5-methylcytosine, 5-(C₃-C₆)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophlysica Acta 1489:117-139 (1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-O, 3′-C-linked [3.2.0] bicycloarabinonucleosides (see e.g. N. K Christiensen, et al., J. Am. Chem. Soc., 120: 5458-5463 (1998). Such analogs include synthetic nucleosides designed to enhance binding properties, e.g., duplex or triplex stability, specificity, or the like.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). Preferably a minimum length for a hybridizable nucleic acid is at least about 12 nucleotides; preferably at least about 18 nucleotides: and more preferably the length is at least about 27 nucleotides; and most preferably 36 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence. Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For example, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

An “enhancer,” as used herein, refers to a polynucleotide sequence that enhances transcription of a gene or coding sequence to which it is operably linked. A large number of enhancers, from a variety of different sources are well known in the art and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

A “detectable marker gene” is a gene that allows cells carrying the gene to be specifically detected (e.g., distinguished from cells which do not carry the marker gene). A large variety of such marker genes are known in the art. Preferred examples thereof include detectable marker genes which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., WO 92/08796, published May 29, 1992, and WO 94/28143, published Dec. 8, 1994).

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) [Reeck et al., Cell, 50:667 (1987)].

Accordingly, the term “sequence similarity” in all its grammatical forms refers to the degree of identity or correspondence between nucleic acid or amino acid sequences of proteins that do not share a common evolutionary origin [see Reeck et al., 1987, supra]. However, in common usage and in the instant application, the term “homologous,” when modified with an adverb such as “highly,” may refer to sequence similarity and not a common evolutionary origin.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Methods of alignment of sequences for comparison are well known in the art. Optimal alignment of sequences for comparison may be conducted by the local homology algorithm of Smith and Waterman, Adv. Appl. Math. 2: 482 (1981), by the homology alignment algorithm of Needleman and Wunsch J. Mol. Biol. 48: 443 (1970), by the search for similarity method of Pearson and Lipman, Proc. Natl. Acad. Sci. USA 85: 2444 (1988), by computerized implementations of these algorithms (including, but not limited to CLUSTAL in the PC/Gene program by Intelligenetics, Mountain View, Calif., GAP, BESTFIT, FASTA, and TFASTA in the Wisconsin Genetics Software Package, Genetics Computer Group (GCG), 575 Science Dr., Madison, Wis., USA), or by inspection. In particular, methods for aligning sequences using the CLUSTAL program are well described by Higgins and Sharp in Gene, 73: 237-244 (1988) and in CABIOS 5: 151-153 (1989)).

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic”, “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target genes. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, the term “mRNA” means the presently known mRNA transcript(s) of a targeted gene, and any further transcripts which may be elucidated.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

The term “high expression” of urease by the recombinants cells and/or compositions of the invention refers to expression levels of about 2 to up to 5 Units per milligram of protein. As defined herein, “1 Unit” is the amount of enzyme needed to cleave one micromole of ammonia in 1 minute.

Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001, and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc., 103:3185, 1981. Chemical synthesis of nucleic acids can be performed, for example, on commercial automated oligonucleotide synthesizers.

Those of skill in the art will be able to prepare suitable vectors starting with widely available vectors which will be modified by genetic engineering techniques known in the art, such as those described by Sambrook et al (Molecular cloning: a Laboratory Manual; 1989). Vectors of the invention are fully described in the examples which follow.

A vector typically comprises one or more origins of replication so that it can be replicated in a host cell, such as bacterial cell or yeast cell (this enables constructs to be replicated and manipulated, for example in E. coli, by standard techniques of molecular biology). A vector also typically comprises at least the following elements, usually in a 5′ to 3′ arrangement: a promoter for directing the expression of a nucleic acid sequence encoding an enzyme of the invention; optionally a regulator of the promoter, a transcription start site, a translational start codon; and a nucleic acid sequence encoding an enzyme, transporter or other desired product of the invention.

The vector may also contain one or more selectable marker genes, for example one or more antibiotic resistance genes. Such marker genes allow identification of transformants. Optionally, the vector may also comprise an enhancer for the promoter. The vector may also comprise a polyadenylation signal, typically 3′ to the nucleic acid encoding the enzyme of the invention. The vector may also comprise a transcriptional terminator 3′ to the sequence encoding the enzyme of the invention.

The vector may also comprise one or more introns or other non-coding sequences, for example 3′ to the sequence encoding the polypeptide of interest. In a typical vector, the nucleic acid sequence encoding the desired enzyme system of the invention is operably linked to a promoter capable of expressing the sequence. “Operably linked” refers to a juxtaposition wherein the promoter and the nucleic acid sequence encoding the desired polypeptide are in a relationship permitting the coding sequence to be expressed under the control of the promoter. Thus, there may be elements such as 5′ non-coding sequence between the promoter and coding sequence. Such sequences can be included in the vector if they enhance or do not impair the correct control of the coding sequence by the promoter.

Any suitable promoter capable of directing expression of the nucleic acid encoding the therapeutic polypeptide or protein may be included in the vector. For example, the promoter may be a bacterial, eukaryotic or viral promoter. The promoter may be constitutive or inducible.

Thus, the invention provides host cells comprising one or more recombinant nucleic acids of the invention. These cells are typically transformed or transfected with the recombinant nucleic acid. The recombinant nucleic acid may be introduced into the host cell by any suitable means. For example, sequences encoding enzymes of the invention may be packaged into infectious viral particles in order to transfect cells. The nucleic acid sequences encoding desired polypeptides may also be introduced by electroporation, lipofection, biolistic transformation or by simply contacting the nucleic acid sequences with cells in solution.

Such vectors may replicate either after integration into the host cell genome or remain extrachromosomal, as in the case of plasmids. Any suitable host cell may be used, including both prokaryotic and eukaryotic microbes.

Recombinant clones may be selected using generally available techniques, such as screening for the presence of a marker. One suitable screening methods include nucleic acid hybridization, antibody assays and plate assays for the detection of protein activity. Secreted products may be recovered from the growth medium by conventional techniques. Alternatively, it can be recovered from the host cells by disrupting them, and then using recovery techniques known in the art.

Recombinant Bacteria Expressing Urease Enzymes and Nickel Transporters

In a preferred embodiment, recombinant ammonia producing cells are stable and produce high levels of urease in the absence of exogenous nickel. Preferably, integration vectors include, but not limited to the mannitol operon of S. mutans UA159 which is targeted as the insertion sight. (The target gene cluster is annotated as smu1082 in the Oral Pathogens database (http://www.stdgen.lanl.gov/oragen/). Mannitol is only poorly cariogenic and in most cases is not a major constituent of the human diet. It is rather poorly metabolized by S. mutans, therefore, loss of mannitol utilization capabilities does not affect competitive fitness of the organism in vivo.

In another preferred embodiment, the insertion vector allows for a double-crossover recombination into the mtl gene cluster so that genes can be integrated into the chromosome in single copy and no selective pressure is needed to maintain the genes in culture medium or in a mammalian host. The genes can be tagged with a variety of antibiotic resistance determinants to track integration and the organisms acquire a phenotype where they are unable to grow on mannitol as the sole carbohydrate source.

In another preferred embodiment, the entire intact urease gene cluster, including ureIABCEFDGMQO are spliced together and placed under the control of the intact urease cognate promoter. However, any strong promoter can be used. In accordance with the invention, the entire functional urease gene cluster can be stably integrated into a host cell genome such as S. mutans at the mtl locus. These strains, as described in detail in the examples which follow expressed a functional urease in the absence of exogenous nickel, as did E. coli carrying the ureI-O genes.

In another preferred embodiment, a strong promoter such as for example, from a tetracycline resistance cassette, drives expression of the entire operon. This new strain of S. mutans produces very high levels of the urease enzyme as compared to the highly ureolytic Streptococcus salivarius 57.I from which the urease genes were derived. No supplementation with nickel is necessary for high activity.

In other preferred embodiments, the recombinant alkali-producing bacteria of the invention comprise a nucleic acid construct that includes a gene cluster encoding a urease enzyme. Studies have shown that the production of ammonia and CO₂ from urea hydrolysis by the enzyme urease has a major impact on microbial pathogenesis. Any suitable prokaryotic or eukaryotic urease enzyme can be expressed. In general, ureases are Ni²⁺-requiring metalloenzymes. Bacterial genes required for the biogenesis of ureases are generally arranged as operons. Typically, the structural genes ure C, B and A encode the α, β and γ subunits, respectively. These are followed by the accessory genes ureE, F, G and D, which encode proteins essential for the incorporation of Ni²⁺ into the metallocenter. Although Ni²⁺ is an essential cofactor for the catalytic activity of ureases, most known ure operons do not comprise genes encoding proteins for Ni²⁺ transport.

The nucleic constructs described herein are preferably isolated from bacterial strains that colonize dental plaque. Streptococcus salivarius is an exemplary ureolytic microorganism that is abundant in the oral cavity. However, its usefulness as a source of isolated nucleic acids for transfer into nonureolytic strains has been limited by the inability of an isolated ureABCEFGD gene cluster, along with a partial ureI from this strain, to direct assembly of a functional urease enzyme. The enzyme in this case is known to be active only in the presence of exogenous NiCl₂. Because free nickel can be toxic, the addition of nickel for stimulating the enzyme may be impractical in a clinical application, such as a dental treatment.

The embodiments disclosed herein, provide alkali producing recombinant cells such as urease-expressing recombinant bacteria, which overcome the need to provide exogenous nickel by including at least one gene for a prokaryotic nickel transporter in the nucleic acid construct used to transform the bacteria. Any suitable combination of alkali producing enzymes, for example urease enzymes, and nickel transporters can be expressed by the recombinant cells.

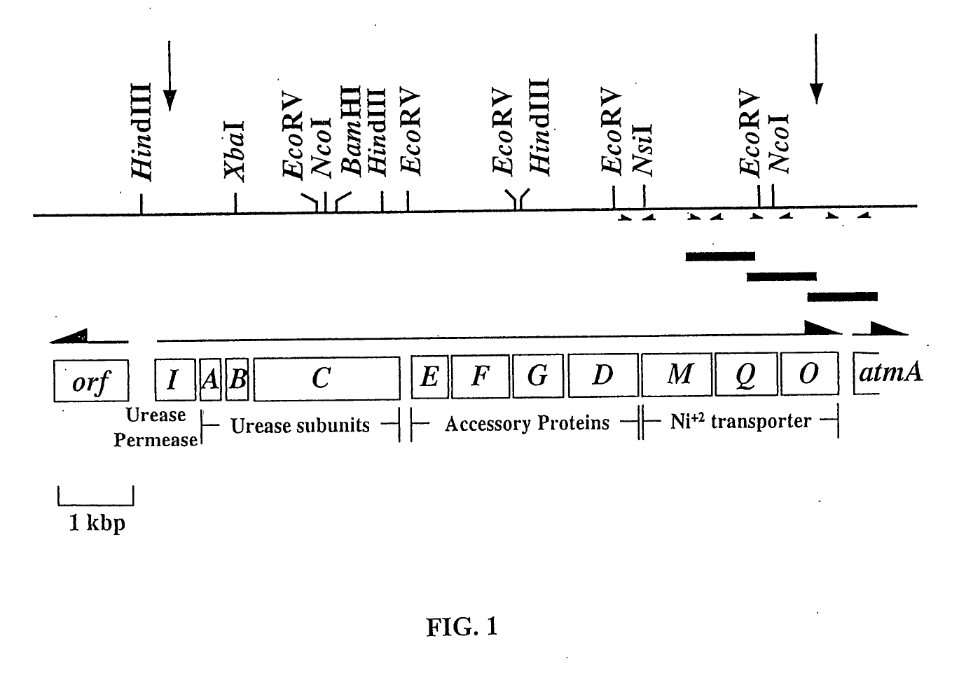

As an illustrative example, not meant to limit or construe the invention in any way, is as follows. A recombinant cell which includes a nickel transporter is a recombinant S. mutans. The recombinant cell further expresses ureI, ureABCEFGD and ureMQO. The expression vectors further comprise a new nickel-specific transport system which we discovered in Streptococcus salivarius 57.I, and show to be included in the urease operon in this strain. Pathogenic microorganisms generally possess more than one nickel uptake system for all nickel-requiring enzymes. By contrast, S. salivarius is known to possess only one nickel-specific uptake system. By searching the partial genome sequence of Streptococcus thermophilus LMG18311, three open reading frames (ORFs 1, 2, 3) homologous to those encoding proteins involved in cobalamin biosynthesis and cobalt transporter (cbiMQO) were identified immediately 3′ to the ure operon. Contiguous transcripts were detected between ureD, and ORF 1, 2 and 3, indicating that the ORFs were part of the ure operon. Further studies demonstrated that ORFs 1, 2, and 3 were indeed part of ure operon, and they were designated tire M, Q and O. Insertional inactivation of ureM completely abolished urease activity and the ability to accumulate ⁶³Ni²⁺ during growth. The ure M, Q and O genes of Streptococcus salivarius 57.I were demonstrated to encode a Ni²⁺-specific ATP-binding cassette transporter. Results of these studies showed that the complete sequence of the ure operon of S. salivarius 57.I consists of 11 genes (ureIABCEFGDMQO), shown schematically in FIG. 1. The complete nucleotide sequence of the ure operon of S. salivarius 57.I is presented herein as SEQ ID NO: 1.

In other preferred embodiments, nucleic acid expression vectors comprise nucleic acid sequences which are about 50% homologous to SEQ ID NO: 1; preferably, the nucleic acid expression vectors comprise nucleic acid sequences which are about 75% homologous to SEQ ID NO: 1; more preferably, the nucleic acid expression vectors comprise nucleic acid sequences which are about 80% homologous to SEQ ID NO: 1; more preferably, the nucleic acid expression vectors comprise nucleic acid sequences which are about 85% homologous to SEQ ID NO: 1; more preferably, the nucleic acid expression vectors comprise nucleic acid sequences which are about 90% homologous to SEQ ID NO: 1; more preferably, the nucleic acid expression vectors comprise nucleic acid sequences which are about 95%, 96%, 97%, 98%, 99%, and 99.9% homologous to SEQ ID NO: 1.

In accordance with the invention, any suitable nickel transporter can be used. For example, two distinct high-affinity nickel transport systems have been described in prokaryotes. The first is the group of single-component Ni²⁺ permeases, which belong to the nickel/cobalt transporter (NiCoT) family. The best known of these is HoxN from Ralstonia eutropha. Similar systems have been identified in other bacteria, including HupN from Bradyrhizobium japonicum, Nix A from H. pylori, and possibly UreH from Bacillus sp. TB-90.

The second described group of prokaryotic nickel transporters are the Nik systems, which belong to the ATP-dependent binding cassette (ABC) transporter family. This type of Ni²⁺-specific transporter is invariably associated with ureolytic activity in the respective microorganisms. Nickel uptake systems of Nik type have been identified in a variety of bacterial strains, including E. coli, Brucella suis, Vibrio parahaemolyticus, Actinobacillus pleuopneumoniae, and Yershina pseudotuberculosis. The nik operon is comprised of several subunits: one periplasmic Ni²⁺ binding protein (i.e., Nik A), two hydrophobic transmembrane proteins (i.e., Nik B and NikC) which are thought to form the channel for Ni²⁺ uptake, and two membrane-associated components (i.e., Nik D and Nik E) which comprise all of the conserved signature sequences characteristic of ATPases. Nik D and E are believed to be involved in the energy-coupling process for transport.

Other nickel transporting genes can be identified. For example, known nickel transporter gene sequences may be used to isolate mutant nickel transporter gene sequences, preferably from an oral colonizing bacterium in a human subject. Human subjects can be classified as those suffering from or susceptible to dental plaque and normal controls, i.e. those individuals which do not have dental plaque as identified by a practitioner.

DNA isolated from bacterial cells of normal and affected individuals can also be used as PCR template. PCR products from normal and affected individuals are compared, either by single strand conformational polymorphism (SSCP) mutation detection techniques and/or by sequencing. The mutations responsible for the loss or alteration of function of the nickel transporter gene product can then be ascertained.

In another embodiment of the invention, the above nucleic acid sequences encoding nickel transporters may be used to generate hybridization probes useful in mapping the naturally-occurring genomic sequence, as well as to detect in an individual, or group of individuals, allelic variants of genes that are present in bacteria from the oral cavity of individuals suffering from or susceptible to dental plaque or other bacterial-mediated diseases. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (see, e.g., Harrington et al., 1997, Nat Genet. 15: 345-355; Price, 1993, Blood Rev. 7: 127-134; and Trask, 1991, Trends Genet. 7: 149-154).

An “allele” or “variant” is an alternative form of a gene. Of particular utility in the invention are variants of the genes encoding any potential nickel transporter related molecule in bacteria colonizing the oral cavity. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

Preferably, the plurality of nucleic acids from different taxonomic species which have homology to the target nucleic acid, as described above in the sequence similarity search, are further delineated so as to find orthologs of the target nucleic acid therein. An ortholog is a term defined in gene classification to refer to two genes in widely divergent organisms that have sequence similarity, and perform similar functions within the context of the organism. In contrast, paralogs are genes within a species that occur due to gene duplication, but have evolved new functions, and are also referred to as isotypes. Optionally, paralog searches can also be performed. By performing an ortholog search, an exhaustive list of homologous sequences from as diverse organisms as possible is obtained. Subsequently, these sequences are analyzed to select the best representative sequence that fits the criteria for being an ortholog. An ortholog search can be performed by programs available to those skilled in the art including, for example, Compare. Preferably, an ortholog search is performed with access to complete and parsed GenBank annotations for each of the sequences. Currently, the records obtained from GenBank are “flat-files”, and are not ideally suited for automated analysis. Preferably, the ortholog search is performed using a Q-Compare program. Preferred steps of the Q-Compare protocol are described in the flowchart set forth in U.S. Pat. No. 6,221,587, incorporated herein by reference.

Preferably, interspecies sequence comparison is performed using Compare, which is available and known to those skilled in the art. Compare is a GCG tool that allows pair-wise comparisons of sequences using a window/stringency criterion. Compare produces an output file comprising points where matches of specified quality are found. These can be plotted with another GCG tool, DotPlot.

The polynucleotides of this invention can be isolated using the technique described in the experimental section or replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) or MacPherson et al. (1991) and (1994), supra, and references cited therein. Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

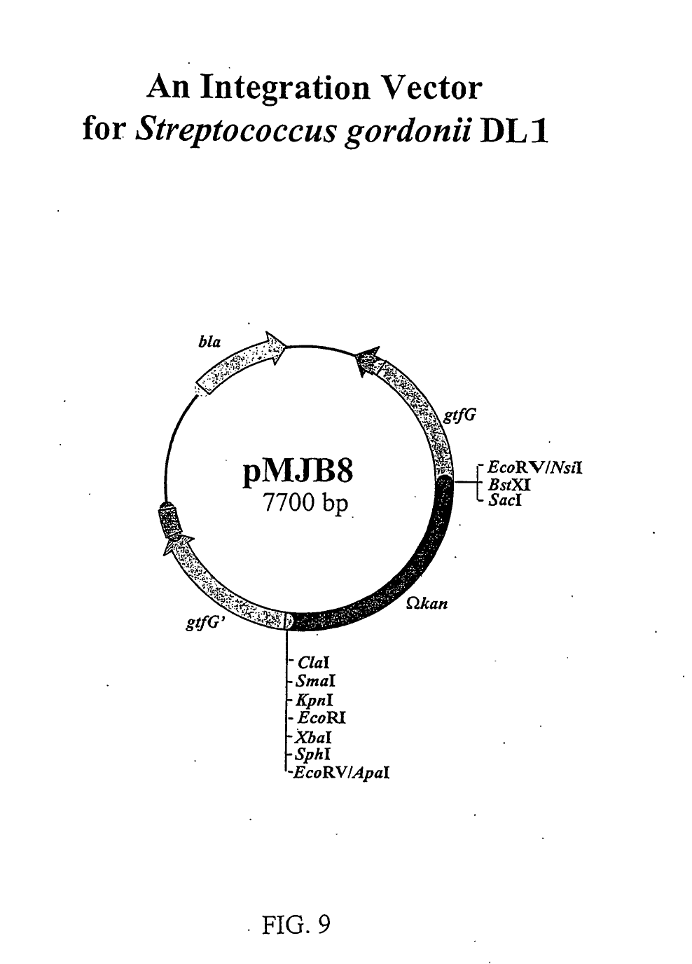

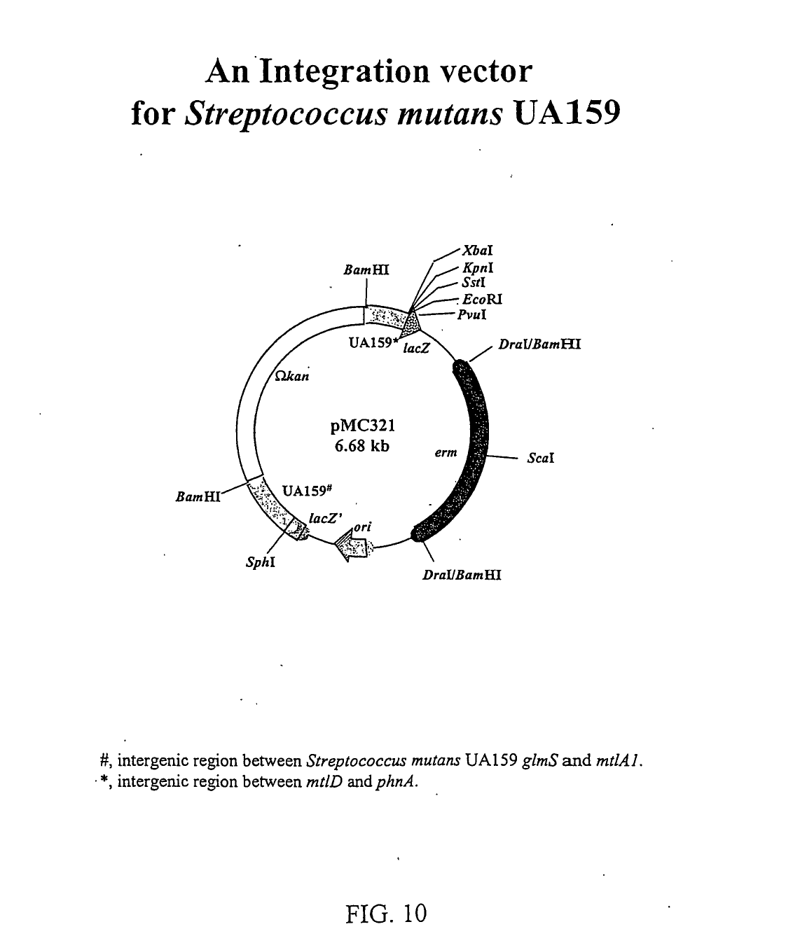

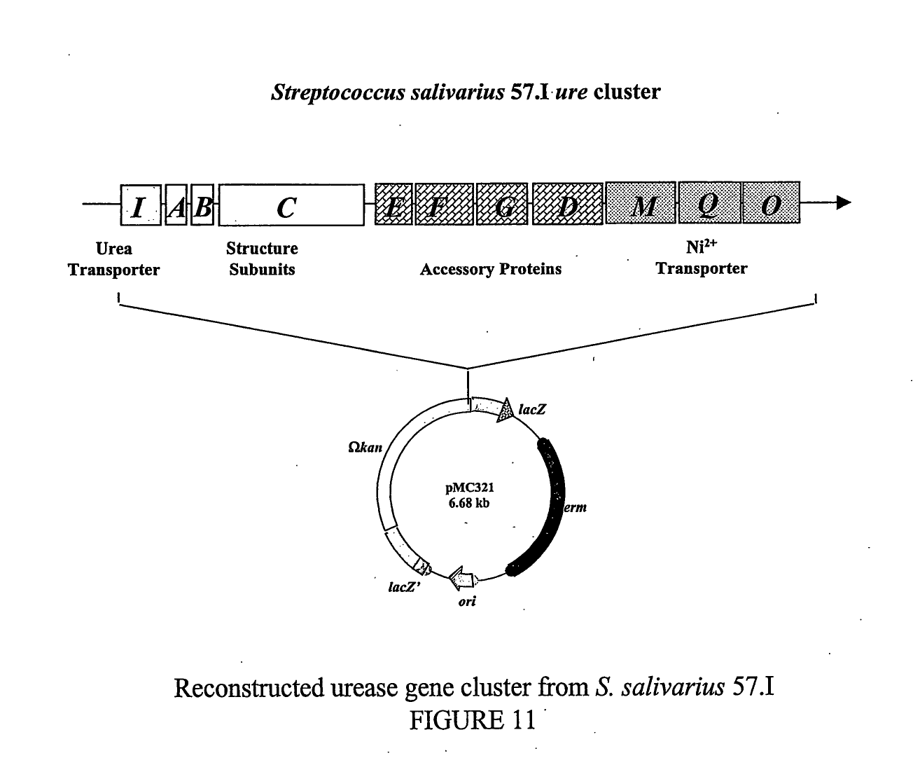

In a preferred embodiment, the recombinant alkali-producing bacteria, which express the selected nucleic acid constructs, are prepared according to techniques well known to those of skill in the art. Suitable methods are further described in the examples below. In general, an integration vector is selected that allows for stable establishment of the foreign genes into the appropriate bacterial strain. Typically, the foreign DNA is integrated into a non-essential gene whose insertional inactivation does not interfere with functional activities of the host bacteria, such as colonization. For example, as further described below, for insertion of genes into plaque bacteria S. mutans, suitable gene loci include gtfA and the mannitol operon. For transformation of S. gordonii, the gtfG locus can be used. Examples of preferred integration vectors including these loci are described herein, and illustrated in FIGS. 9 and 10.

In a preferred embodiment, genetic loci from any oral colonizing bacteria can be used. The invention does not limit the loci to gft clusters. Preferred strains produce high expression levels of an alkali producing enzyme. High expression of alkali are determined by the procedures described in detail in the examples which follow.

Recombinant Bacteria Expressing Arginine Deiminase (AD)

In other preferred embodiments, the recombinant bacteria comprise a nucleic acid construct that includes a gene cluster encoding an arginine deiminase system. The arginine deiminase system (ADS) is one of two major ammonia-generating pathways in the oral cavity known to play an important role in maintaining pH homeostasis and ecology in the oral biofilm. The prokaryotic ADS is a three-enzyme pathway that catalyzes the conversion of arginine to ornithine, ammonia and CO₂ with concomitant production of ATP. Arginine is first hydrolyzed to AD, to generate citrulline and ammonia. Citrulline is then converted to ornithine and carbamylphosphate via a catabolic ornithine carbamyltranferase (cOTC). Finally, carbamate kinase (CK) transfers phosphate from carbamylphosphate to ADP to produce ATP, CO₂, and ammonia. In some cases, an arginine-ornithine antiporter which catalyzes the uptake of arginine and concomitant export of ornithine, and a putative transaminase or peptidase have been found to be part of arc gene clusters.

The AD-expressing bacteria of the invention are preferably isolated from strains that colonize the oral cavity. Comparatively few ADS-positive organisms colonize the mouth. Of these, Streptococcus gordonii is preferred because it is one of the most abundant organisms in tooth biofilms. Furthermore, S. gordonii, belonging to the viridans group of streptococci, is among the microorganisms known to be early colonizers of teeth that make up a major portion of healthy human supragingival dental plaque. The ability of S. gordonii to produce ammonia via the ADS pathway is thought to contribute to the inhibition of colonization of cariogenic bacteria.

Another preferred strain of AD-expressing bacteria that colonizes the oral cavity is Streptococcus rattus. S. rattus is similar to other ADS-positive oral bacteria in that it is relatively acid sensitive in the absence of arginine and has not been linked to caries development in humans. However, S. rattus is a member of the mutans group of streptococci and is most closely related to S. mutans, which is noted for exceptional cariogenicity and acid tolerance. A major factor distinguishing S. rattus from S. mutans is the ability to catabolize arginine via the ADS. Accordingly, the presence of the ADS in S. rattus may have critical implications for the pathogenicity of mutans streptococci.

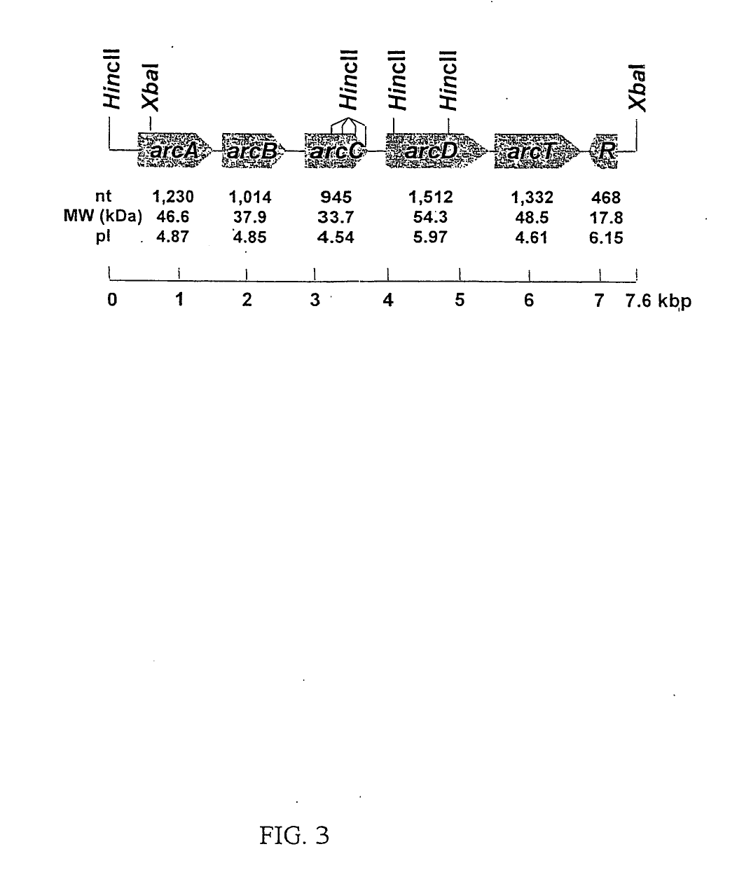

The ADS gene cluster was isolated from both S. gordonii and S. rattus, as described in the examples which follow. Briefly, subgenomic DNA libraries of S. gordonii DL1 were screened using a probe specific for arcB, the gene that encodes catabolic ornithine carbamyltransferase (cOTC). Nucleotide sequence analysis of the ADS gene cluster of S. gordonii DL1 revealed six open reading frames (ORFs) that were arranged contiguously. The first five ORFs were transcribed in the same direction, as an apparent operon, and the sixth was transcribed in the opposite direction. The ORFs were found to share significant homologies and to correspond closely in molecular mass to previously characterized arc genes. The arrangement of these ORFs, designated arcA (AD), arcB (ornithine carbamyltransferase), arcC (carbamate kinase), arcD (arginine-ornithine antitransporter), arcT (dipeptidase), and arcR (regulator) is shown schematically in FIG. 3. The complete nucleotide sequence of the AD gene cluster from S. gordonii, including an anaerobic regulator, flp, is presented herein as SEQ ID NO:2.

A putative σ⁷⁰ promoter ParcA was mapped 5′ to arcA by primer extension, and the expression of ParcA was shown to be inducible by arginine and repressible by glucose, in agreement with AD specific activities measured in the wild-type strain. Studies with an ArcR-deficient strain confirmed that ArcR is a transcriptional activator that is required for induction and optimal expression of the S. gordonii gene cluster.

For isolation and analysis of the AD gene cluster in S. rattus FA-1, an arcB gene fragment was obtained by degenerate PCR, and used to identify the FA-1 arc operon in subgenomic DNA libraries. The results showed that the genes encoding the arginine deiminase pathway in S. rattus FA-1 are organized as an arcABCDTR operon, including the enzymes of the pathway, as well as an arginine:ornithine antiporter and a putative regulatory protein. The complete nucleotide sequence of the AD operon of S. rattus is disclosed herein as SEQ ID NO:3. A σ⁷⁰-like promoter was mapped 5′ to arcA by primer extension, and reverse transcriptase PCR was used to establish that arcABCDT could be co-transcribed. Reporter gene fusions and AD assays demonstrated that the operon is regulated by substrate induction and catabolite repression, the latter apparently through a CcpA-dependent pathway.

Other sources for cloning of AD include such micro-organisms which are rich in arginine deiminase. Examples include: Lactobacillus acidophilus, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus lensenii, Lactobacillus leichmanii, Lactobacillus minutus, Lactobacillus plantarum, Lactobacillus rogosae, Lactobacillus salivarius, Bifidobacterium adolescentis, Bifidobacterium angulatuin, Bifidobacterium bifidum, Bifidobacterium breve, Bifidobacterium catenulatum, Bifidobacterium dentium, Bifidobacteriuin eriksonii, Bifidobacterium infantis, Bifidobacterium longuin, Bifidobacterium plantarum, Bifidobacterium pseudo-catenulatum, Bifidobacterium pseudolongum, Streptococcus lactis, Streptococcus raffinolactis, Streptococcus thermophilus, Acidaminococcus fermenta, Cytophaga fermentans, Rhodoferax femmentans, Cellulomonas fermentans and Zymomonas mobilis.

Recombinant Bacteria Expressing Agmatine Deiminase (AgDS)

Yet other embodiments of the alkali-producing recombinant bacteria of the invention comprise a nucleic acid construct that includes a gene cluster encoding enzymes of the agmatine deiminase system (AgDS). The AgDS generates putrescine, ATP, carbon dioxide and ammonia from agmatine, a decarboxylated derivative of arginine (Simon and Stalon, 1982). The AgD system has been identified in a broad range of organisms, including maize shoots, rice, soybean, cucumber seedlings, Enterococcus faecalis, Pseudomonas aeruginosa and Lactobacillus hilgardii. The physiological role of the AgDS, as well as the enzymes associated with AgDS, differ among species. In lactic acid bacteria and E. faecalis, the AgDS most closely resembles the arginine deiminase system.

Agmatine enters the cell via an agmatine:putrescine antiporter, where it is hydrolyzed to N-carbamoylputrescine and ammonia by agmatine deiminase (AgD; EC 3.5.3.12). Putrescine carbamoyltransferase (PTC; EC 2.1.3.6) mediates the phosphorolysis of N-carbamoylputrescine, yielding putrescine and carbamoylphosphate. Finally, a phosphate group is transferred from carbamoylphosphate to ADP by carbamate kinase (CK; EC 2.7.2.2), generating ATP, CO₂ and NH₃. Putrescine is then exchanged for agmatine via the antiporter.

In studies disclosed herein, a cluster of genes forming the components of the AgDS was identified in the highly cariogenic bacterium, Streptococcus mutans UA159. These genes, annotated as otcA, SMU.263, SMU.264 and arcC, respectively encode a putative putrescine carbamoyltransferase, amino acid antiporter, agmatine deiminase and carbamate kinase. These enzymes generate putrescine, ATP, carbon dioxide and ammonia from agmatine. The complete nucleotide sequence of the AgDS operon of Streptococcus mutans UA159, herein designated as aguBDAC, is disclosed herein as SEQ ID NO: 4. The amino acid sequences of the five polypeptides encoded by the genes of the S. mutans agmatine deiminase operon, i.e., SMU.261c LuxR-like transcriptional regulator, putrescine carbamyltransferase, agmatine:putrescine antiporter, agmatine deiminase, and carbamate kinase, are listed herein as SEQ ID NOS:46-50.

RNA analyses and enzymatic assays in S. mutans demonstrated that expression of the system is in the presence of agmatine and that the genes are regulated by carbohydrate catabolite repression (CCR). It was shown that S. mutans is capable of producing ammonia from agmatine at pH values as low as 4, suggesting that ammonia production by this pathway provides an ancillary acid tolerance mechanism.

Compositions Comprising Recombinant Bacteria

In another aspect, the invention provides compositions, in a suitable carrier, including at least one recombinant bacterial cell comprising an isolated nucleic acid construct wherein the cell expresses at least one ammonia- or other alkali-producing (“alkalinizing”) enzyme. In preferred embodiments, the recombinant cells are applied to the oral cavity by a practitioner. However, the compositions are suitable for self-administration. Each type of recombinant cell can be formulated in doses, such as the number of bacterial cells and preserved for use in different compositions for oral delivery to a subject. Methods for preserving bacterial cells are well known in the art such as, lyophilizing the cells. See for example, Madigan, M. T, J. M. Martinko, and J. Parker. Brock: Biology of Microorganisms, 10th ed. Prentice-Hall, Inc. Englewood Cliffs, N.J. 2003.

The alkalis can include ammonia and its derivatives, and other organic amines. In some embodiments of the compositions, the nucleic acid construct includes an operon encoding a urease enzyme and a nickel transporter. In other embodiments, a gene cluster encoding an arginine deiminase system or an agmatine deiminase system is included.

The compositions can be formulated in any suitable carrier. For use in dental applications, a suitable carrier can include but is not limited to chewing gum, toothpaste, a lozenge, a powder, a gel, an ointment, a cream, a liquid, a mouthwash, a rinse or a candy. The term “oral composition” as used herein means the total composition that is delivered to the oral surfaces. The composition is further defined as a product which, during the normal course of usage, is not, the purposes of systemic administration of particular therapeutic agents, intentionally swallowed but is rather retained in the oral cavity for a time sufficient to contact substantially all of the dental surfaces and/or oral tissues for the purposes of oral activity. Examples of such compositions include, but are not limited to, toothpaste, dentifrice, mouthwash or mouth rinses, topical oral gels, denture cleanser, mouth spray, dental floss, confectionery including chewing gum and lozenge, and the like.

The term “dentifrice” as used herein means paste, gel, or liquid formulations unless otherwise specified. The dentifrice composition can be in any desired form such as deep striped, surface striped, multi-layered, having the gel surrounding the paste, or any combination thereof. Alternatively the oral composition may be dual phase dispensed from a separated compartment dispenser.

The term “confectionery” as defined herein means a solid, gum, gum-like, or glassy composition optionally having a liquid center filling and/or optionally coated which comprises greater than about 25% sugar or sugar alcohol. Such compositions usually have a sweet taste. Examples of confectionery products include, but are not limited to, breath mints, low boiled candy, chewing gum, hard boiled candy, coated candy, lozenges, pressed mints, throat drops and the like.

The term “chewing gum” as defined herein means a confectionery composition which is suitable for chewing and which comprises 2% or greater, by weight of the composition, of elastomer.

The term “gum base” as defined herein means a material or mixture of materials which is used in confectionery composition but which comprises a non-digestible elastomer, plastic or resin. The term “elastomer” as defined herein means a non-digestible polymeric material, or mixture of materials, such as the materials typically used in chewing gum compositions.

Active and other ingredients useful herein may be categorized or described herein by their cosmetic and/or therapeutic benefit or their postulated mode of action. However, it is to be understood that the active and other ingredients useful herein can in some instances provide more than one therapeutic and/or cosmetic benefit or operate via more than one mode of action. Therefore, classifications herein are made for the sake of convenience and are not intended to limit an ingredient to the particularly stated application or applications listed.

Compositions of the present invention comprise a carrier material into which other ingredients are solubilized, dispersed or otherwise mixed. Depending upon the type of composition in question the carrier material can differ. For example mouth wash compositions commonly have a carrier material which comprises from about 20:1 to about 2:1 aqueous alcoholic matrix; dentifrice compositions usually comprise an aqueous matrix system; denture cleansers which are usually hard pressed tablets, dental floss where the carrier is a fiber or paper material and confectionery compositions wherein the carrier material is a sweetener matrix. The preferred compositions of the present invention are dentifrice, mouthwash and confectionery compositions including chewing gum. It is preferred that non-confectionery compositions of the present invention are single phase compositions by which is meant that the whole composition is stably stored within a single container. The most important elements of the carrier systems for these products are discussed below.

Water

Water used in the preparation of commercially suitable compositions should preferably be of low ion content and free of organic impurities. The amount of water in a composition should be considered to be not only that added as free water, but also water, which is introduced with other materials, such as with sorbitol, silica, surfactant solutions and or color solutions. Compositions of the present invention can comprise water from about 0.1% to about 99%, preferably from about 0.5% to about 50%, by weight of the composition. It is highly preferred that compositions of the present invention comprises less than about 10%, preferably less than about 8%, more preferably less than about 5%, even more preferably less than about 3%, and most preferably less than about 2%, by weight of the composition, water.

Confectionery Carrier Material

Compositions of the present invention are preferably confectionery compositions including chewing gum. Suitable physical forms include sticks, dragees, chicklets, and batons. Although the exact ingredients for each product form will vary from product to product, the specific techniques will be known by one skilled in the art. However there are some general ingredients, which are common to all product forms and these are discussed in more detail below. Preferred product forms are chocolate bars, pressed tablets, low boiled candy, hard boiled candy and chewing gum which are readily formulated with less than about 10%, by weight of the composition, water.

Confectionery compositions of the present invention comprise a carrier material. The carrier materials vary depending on the type of confectionery used and would be well known to one skilled in the art. The carrier material can be chosen from chewable or non-chewable materials. It is preferred that the compositions comprise at least 10% chewable material. The chewable material can be selected from gums including agar gum, gelatin etc; low boiled sugar candy base and gum base materials. It is preferred that the carrier material for compositions of the present invention are not in the form of a whipable or aerated emulsion. Hard and low boiled candy carrier, pressed tablets and the like usually comprise greater than about 70% bulk sweetener including suitable sugar and sugar syrups including cariogenic and non-cariogenic materials. Low boiled candies can also comprise butter to form chewable toffee. For jelly and gum drop compositions the carrier comprises greater than about 25% bulk sweetener and additionally comprise gums including gum arabic, gelatin, agar powder and the like.

Compositions of the present invention can be in the form of a chewing gum. As such it is preferred that the compositions comprise greater than about 10%, preferably greater than about 15%, more preferably greater than about 20% and most preferably greater than about 25%, up to about 75%, by weight of the composition, of gum base. The gum base comprises a carrier material, or mixture of carrier materials, selected from elastomers, resins or waxes. The gum base carrier materials are water insoluble materials which are typically not released in the mouth.

Such materials include:

(i) Elastomers and Elastomer Solvents. Compositions of the present invention can comprise an elastomer, or mixture of several different elastomers. Elastomeric materials are generally known in the art but illustrative examples include styrene-butadiene rubber (SBR); synthetic gums; polyisobutylene and isobutylene-isoprene copolymers; natural gums; chicle; natural rubber; jelutong; balata; guttapercha; lechi caspi; sorva; and mixtures thereof. Compositions of the present invention preferably comprise from about 2% to about 30%, more preferably from about 5% to about 25%, by weight, of elastomer. These levels are determined by the desired final texture of the chewing gum since when the total level of elastomer is below about 2% the base composition lacks elasticity, chewing texture, and cohesiveness whereas at levels above about 30% the formulation is hard, rubbery and maintains a tight chew. Elastomer solvents are also preferably present in compositions of the present invention since they aid softening of the elastomer component. Preferred examples of elastomer solvents for use herein include the pentaerythritol ester of partially hydrogenated wood rosin, pentaerythritol ester of wood rosin, glycerol ester of partially dimerized rosin, glycerol ester of polymerised rosin, glycerol ester of tall oil, wood or gum rosin, glycerol ester of partially hydrogenated rosin, methyl ester of partially hydrogenated rosin, and mixtures thereof. Compositions of the present invention preferably comprise from about 2% to about 50%, more preferably from about 10% to about 35%, by weight, of elastomer solvent.

(ii) Resins and Waxes Resins are an optional, but desirable, ingredient of chewing gum compositions herein. They serve to plasticise the gum base. Suitable resins include polyvinyl acetate (PVA); terpene resins, including polyterpene and polymers of alpha-pinene or beta-pinene; and mixtures thereof. Such compositions preferably comprise from about 3% to about 25%, preferably from about 5% to about 20%, by weight, of resin. The chewing gum compositions may also include one or more waxes. Suitable waxes include paraffin wax; microcrystalline wax; Fischer-Tropsch paraffin; natural waxes such as candellilla, carnauba and beeswax; polyolefin waxes such as polyethylene wax; and mixtures thereof. Compositions comprise up to about 25%, preferably from about 5% to about 20%, by weight, of wax.

Confectionery compositions of the present invention can be center filled. Such products preferably comprise from about 60% to about 95%, more preferably from about 75% to about 85% of an edible shell and from about 5% to about 40%, preferably from about 15% to bout 25%, by weight of the composition, of an edible filling. It is possible that center filled confectionery composition can comprise a suitable medium for preserving the recombinant cells, such as flavored glycerol but would not include agents that are toxic to the patient. In addition the composition can comprise different flavoring agents in the shell and the filling.

Compositions of the present invention may comprise one or more crunchy solid particles dispersed throughout the carrier material. The crunchy preferably particle has a minimum particle size such that the particles are retained by a 0.1 mm mesh, preferably a 0.112 mm mesh, more preferably a 0.16 mm mesh, even more preferably a 0.18 mm mesh and most preferably a 0.2 mm mesh wherein the meshes are selected from the DIN 4188 mesh series. Furthermore the particle preferably has a maximum particle size such that it passes through a 2 mm mesh, preferably a 1 mm mesh, more preferably a 0.8 mm mesh, even more preferably a 0.5 mm mesh and most preferably a 0.4 mm mesh, again wherein the meshes are selected from the DN 4188 mesh series. The solubility of the particle is preferably at least 1 g per 100 ml at 25° C., more preferably at least 5 g, even more preferably at least 8 g and most preferably at least 15 g per 100 ml at 25° C. Finally it is preferred that the particulate material has a hardness of greater than 1, preferably greater than 2 on the Mohs hardness scale. The particle size, solubility and hardness properties confer a crunchy texture to the confectionery itself. Such particles can be present as solid forms of one of the oral care actives outlined above, in the case where the oral care active is a solid, or can be a further particle such as sugar crystals, dried fruits, nuts, etc. Preferably the crunch is provided by the polyphosphate particles. The crunchy texture can be used to reinforce the oral care benefits to the consumer. Different crunchy textures can be obtained by milling the particles to the desired size or by blending different commercial grades of particles to achieve the desired crunch. It is preferred the that crunchy sensation remains consumer noticeable for at least 1 minute 30 seconds, preferably for at least 2 minutes and more preferably for at least 2 minutes 30 seconds. However it is also preferred that the crunchy texture has disappeared by 5 minutes, preferably by 4 minutes so that the material does not abrade the dentin or so that the product does not have a gritty residue.

Furthermore the confectionery compositions of the present invention can also be coated. The outer coating may be hard or crunchy. Typically, the outer coating will essentially consist of sorbitol, maltitol, xylitol, isomalt, and other crystallisable polyols. Furthermore the coating will typically consist of several opaque layers, such that the confectionery core is not visible through the coating itself, which can optionally be covered with a further one or more transparent layers for aesthetic, textural and protective purposes. The outer coating may also contain small amounts of water and gum arabic. A polyol coating can be further coated with wax. The coating is applied in a conventional manner by successive applications of a coating solution, with drying in between each coat, as described in WO 99/44436. As the coating dries it usually becomes opaque and is usually white, though other colorants may be added. A polyol coating can be further coated with wax. The coating can further comprise colored flakes or speckles. If the composition comprises a coat it is possible that one or more of the oral care actives can be dispersed throughout the coat. This is especially preferred if one or more oral care active is incompatible in a single phase composition with another of the actives.

Balance of the Composition

Compositions of the present invention preferably comprise safe and effective levels of one or more additional components. Such materials are well known and are readily chosen by one skilled in the art based on the oral care, physical and aesthetic properties desired for the compositions being prepared. Examples of such materials include, but are not limited to fats, solvents, waxes, emulsifiers, softeners, bulking agents, cationic material, buffers, whitening agents, alkali metal bicarbonate salts, thickening materials, humectants, water, surfactants, titanium dioxide, flavoring agents, coloring agents, and mixtures thereof.

Sweeteners

Two main types of sweeteners exist; bulk sweeteners and high intensity sweeteners. In general, the amount of sweetener used will vary depending on the sweetener and the overall desired aesthetics but levels used should be high enough such that the desired level of sweetness is achieved independent from the flavor. When bulk sweeteners are used they can also assume the role of the bulking agent or filler within the composition.

Bulk Cariogenic Sweetener: Compositions of the present invention may comprise sweetener materials. Such materials include monosaccharides, disaccharides, polysaccharides and mixtures thereof. Examples include xylose, ribose, glucose, mannose, galactose, fructose, dextrose, sucrose, sugar maltose, fructo oligosaccharide syrups, partially hydrolyzed starch, or corn syrup solids and mixtures thereof. However, such materials can often lead to the formation of cavities since they are readily metabolized by bacteria and other micro-organisms in the oral cavity. It is preferred that compositions of the present invention comprise less than about 10%, preferably less than about 5%, more preferably less than about 2%, even more preferably less than about 1%, and most preferably less than about 0.5%, by weight of the composition, of cariogenic sweetener. Compositions of the present invention may optionally comprise 0% cariogenic sweetener.

Bulk Non Cariogenic Sweeteners: Compositions of the present invention preferably comprise a non-cariogenic sweetener. As used herein the term “non-cariogenic” refers to sweeteners which are not able to be metabolized by oral microbes and therefore do not contribute to the formation of dental caries. It is preferred that compositions of the present invention comprise greater than about 10%, preferably greater than about 20%, more preferably greater than about 30% and most preferably greater than about 40%, by weight of the composition, of non cariogenic sweetener. Compositions of the present invention may optionally comprise up to about 99%, by weight of the composition, non-cariogenic sweetener. Preferred bulk non cariogenic sweetening agents are sugar alcohols such as sorbitol, xylitol, mannitol, maltitol, isomalt, hydrogenated starch hydrolysate, insulin, and other non-cariogenic edible polyols such as glycerin and erythritol and mixtures thereof. Most preferred are non cariogenic sweeteners selected from the group consisting of maltitol, mannitol, xylitol, sorbitol, sucralose, aspartame and its salts, and mixtures thereof. In general compositions comprise from about 10% to about 80%, more preferably from about 30% to about 70%, by weight, of bulk sweetener.

High Intensity Sweeteners: High intensity sweeteners may prolong the flavor of the finished gum composition during chewing. Suitable high intensity sweeteners include: dipeptide based sweeteners such as L-aspartyl-L-phenylalanine methyl ester (Aspartame) and equivalents (described in U.S. Pat. No. 3,492,131), L-α-aspartyl-N-(2,2,4,4-tetramethyl-3-thietanyl)-D-alaninamide hydrate (Alitame) and the like; saccharin and its soluble salts e.g. sodium or calcium saccharin salts; cyclamate salts for example acesulfame-K and the like; chlorinated derivatives of sucrose such as chlorodeoxysucrose and the like; and protein based sweeteners, such as Thaumatin (talin). Compositions of the present invention preferably comprise from about 0.01% to about 2.0%, more preferably from about 0.05% to about 0.5%, by weight, of high intensity sweetener.

Humectants

The compositions of the present invention may comprise humectants which can serve to prevent the composition hardening upon exposure to air. In addition certain humectants can also act as sweeteners. Suitable humectants include glycerin, sorbitol, polyethylene glycol, propylene glycol and other edible polyhydric alcohols. The humectant is generally present in an amount of from about 0.5% to about 70%, preferably from about 15% to about 55%, by weight of the composition.

Thickeners

The present invention provides for compositions in a wide variety of product forms. Typically these compositions comprise some thickening material or binders to ensure that the final composition has the desired consistency. Preferred thickening agents are carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthum gum, gum arabic, and gum tragacanth can be used as part of the thickening agent to further improve the texture. Thickening agents can be used in an amount of from about 0.1% to about 15%, by weight of the composition.

Buffering Agents

The present compositions may comprise a buffering agent. Buffering agents, as used herein, refer to agents that can be used to adjust the pH of the compositions of a range of from about pH 3 to about pH 10. Preferred buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. Buffering agents are used at a level of from about 0.1% to about 30%, preferably from about 1% to about 10% and more preferably from about 1.5% to about 3%, by weight of the composition.

Colors

Coloring agents may also be added to the present composition. The coloring agent may be in the form of an aqueous solution, preferably 1% coloring agent, in a solution of water. Color solutions generally comprise from about 0.01% to about 5%, by weight of the composition.

Flavoring Agents

Compositions of the present invention can preferably comprise a flavoring agent. As used herein the term “flavoring agent” means those flavor essences and equivalent synthetic materials which are added to flavor the composition. The flavoring agent can also include specific materials which are added to provide a warming or cooling sensation. Flavoring agents are well known in the art. They include synthetic flavors and or oils and or essences derived from plants, roots, beans, nuts, leaves, flowers, fruits and so forth and mixtures thereof. Examples of suitable flavors include lemon, orange, banana, grape, lime, apricot, grapefruit, apple, strawberry, cherry, chocolate, pineapple, coffee, cocoa, cola, peanut, almond, liquorice, cinnamon and the like. The amount of flavoring agent employed is normally a matter of preference but in general they are used in amounts up to about 4%, preferably from about 0.1 to about 1%, by weight of the composition. Compositions of the present invention can optionally comprise a cooling agent and suitable materials are described in WO 97/06695. Preferred for use herein are physiological cooling agents selected from the group consisting of menthol, peppermint oil, N-substituted-p-menthane-3-carboxamides, acyclic tertiary and secondary carboxamides, 3-1-methoxy propan-1,2-diol and mixtures thereof. Particularly preferred are menthol and menthol containing oils such as peppermint oil. Cooling agents are preferably used at a level of from about 0.001 to about 5%, more preferably from about 0.05% to about 3.5%, by weight of the composition.

Method for Reducing Acidification of Dental Plaque

In yet another aspect, the invention provides a method for reducing acidification of dental plaque in a subject. As described above, plaque acidification is an undesirable condition correlated with the increased formation of caries in human subjects. In studies disclosed herein, plaque from caries-free control subjects was shown to have approximately three-fold higher ureolytic capacity than plaque from caries-active subjects. An inverse relationship was found between the specific activity of urease in dental plaque and dental caries, despite similar levels of urease activity in the saliva of the two groups of subjects.

In other studies described in the examples below, it was shown, using a 10-species bacterial consortium cultivated to form an oral biofilm in a constant-depth film fermentor, that the inclusion of species that expressed high levels of urease enzymes was required for maintenance of all ten species. Omission of urease-producing bacteria resulted in dramatic loss of biodiversity, with dominance of the biofilm by aciduric species.

In the practice of the method for reducing acidification of dental plaque, a subject having at least one tooth with dental plaque disposed upon it is provided. The tooth is bathed in a fluid environment subject to acidification to a first pH in the acid range, i.e., in the range of about 5.5 to about pH 4, for example, upon glycolysis that follows chewing and degradation of foodstuffs in contact with the bacteria of the plaque biofilm. To prevent or reduce such acidification, in the practice of the invention, the subject's tooth or teeth are contacted with a composition comprising at least one alkalinizing recombinant bacterial cell, such as one that produces an alkali such as ammonia. Ammonia causes an increase in the pH of the fluid environment to a second pH higher than the first pH, the second pH typically being in the range of about 6 to about 7.4. By producing an alkali, the recombinant bacteria alter the pH of the biofilm, promoting an environment unfavorable for the growth of caries-causing bacteria, and favorable for the growth of desirable flora of the tooth film.

Viral Vectors

Various techniques using other vectors such as, viral vectors for the introduction of an ammonia producing enzyme, such as urease, into a cell may be utilized in the methods of the invention. Viral vectors for use in the invention are those that exhibit low toxicity to a host cell and induce production of therapeutically useful quantities of ammonia in a tissue-specific manner. Viral vector methods and protocols that may be used in the invention are reviewed in Kay et al. Nature Medicine 7:33-40, 2001. The use of specific vectors, including those based on adenoviruses, adeno-associated viruses, herpes viruses, and retroviruses are described in more detail below.

The use of recombinant adenoviruses as vectors is discussed in W. C. Russell, Journal of General Virology 81:2573-2604, 2000; and Bramson et al., Curr. Opin. Biotechnol. 6:590-595, 1995. Adenovirus vectors are preferred for use in the invention because they (1) are capable of highly efficient gene expression in target cells and (2) can accommodate a relatively large amount of heterologous (non-viral) DNA. A preferred form of recombinant adenovirus is a “gutless, “high-capacity”, or “helper-dependent” adenovirus vector. Such a vector features, for example, (1) the deletion of all or most viral-coding sequences (those sequences encoding viral proteins), (2) the viral inverted terminal repeats (ITRs) which are sequences required for viral DNA replication, (3) up to 28-32 kb of “exogenous” or “heterologous” sequences (e.g., sequences encoding an ammonia producing enzyme), and (4) the viral DNA packaging sequence which is required for packaging of the viral genomes into infectious capsids. For specifically targeting liver, preferred variants of such recombinant adenoviral vectors contain tissue-specific (e.g., mouth) enhancers and promoters operably linked to for example, a urease gene.

Other viral vectors that might be used in the invention are adeno-associated virus (AAV)-based vectors. AAV-based vectors are advantageous because they exhibit high transduction efficiency of target cells and can integrate into the host genome in a site-specific manner. Use of recombinant AAV vectors is discussed in detail in Tal, J., J. Biomed. Sci. 7:279-291, 2000 and Monahan and Samulski, Gene Therapy 7:24-30, 2000. A preferred AAV vector comprises a pair of AAV inverted terminal repeats which flank at least one cassette containing a tissue (e.g., gum)- or cell-specific promoter operably linked to a urease nucleic acid. The DNA sequence of the AAV vector, including the ITRs, the promoter and, for example, urease gene may be integrated into the host genome.

The use of herpes simplex virus (HSV)-based vectors is discussed in detail in Cotter and Robertson, Curr. Opin. Mol. Ther. 1:633-644, 1999. HSV vectors deleted of one or more immediate early genes (IE) are advantageous because they are generally non-cytotoxic, persist in a state similar to latency in the host cell, and afford efficient host cell transduction. Recombinant HSV vectors can incorporate approximately 30 kb of heterologous nucleic acid. A preferred HSV vector is one that: (1) is engineered from HSV type I, (2) has its IE genes deleted, and (3) contains a tissue-specific promoter operably linked to a urease nucleic acid. HSV amplicon vectors may also be useful in various methods of the invention. Typically, HSV amplicon vectors are approximately 15 kb in length, and possess a viral origin of replication and packaging sequences.

Retroviruses such as C-type retroviruses and lentiviruses might also be used in the invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a urease nucleic acid.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells. Lentiviral vectors for use in the invention may be derived from human and non-human (including SIV) lentiviruses. Preferred lentiviral vectors include nucleic acid sequences required for vector propagation as well as a tissue-specific promoter operably linked to, for example, a urease gene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon. A replicon may contain (1) alphavirus genetic elements required for RNA replication, and (2) a heterologous nucleic acid such as one encoding a urease nucleic acid. Within an alphivirus replicon, the heterologous nucleic acid may be operably linked to a tissue-specific promoter or enhancer.

Recombinant, replication-defective alphavirus vectors are advantageous because they are capable of high-level heterologous (therapeutic) gene expression, and can infect a wide host cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a host cell. The replicons may also exhibit transient heterologous nucleic acid expression in the host cell. A preferred alphavirus vector or replicon is non-cytopathic.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates the secretion of a gene product from the host cell.

To combine advantageous properties of two viral vector systems, hybrid viral vectors may be used to deliver a urease nucleic acid to a target tissue (e.g., gum tissue). Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the host cell genome and effect stable urease gene expression.

Other nucleotide sequence elements which facilitate expression of the ammonia producing genes and cloning of the vector are further contemplated. For example, the presence of enhancers upstream of the promoter or terminators downstream of the coding region, for example, can facilitate expression.

Non-Viral Delivery

In addition to viral vector-based methods, non-viral methods may also be used to introduce an ammonia producing gene into a host cell, such as described in the examples which follow. Alternate methods are described herein. A review of non-viral methods of gene delivery is provided in Nishikawa and Huang, Human Gene Ther. 12:861-870, 2001. For example, a non-viral gene delivery method according to the invention employs plasmid DNA to introduce a urease nucleic acid into a cell. Plasmid-based gene delivery methods are generally known in the art and are described in references such as Ilan, Y., Curr. Opin. Mol. Ther. 1:116-120, 1999, Wolff, J. A., Neuromuscular Disord. 7:314-318, 1997 and Arztl, Z., Fortbild Qualitatssich 92:681-683, 1998.

Methods involving physical techniques for introducing a urease nucleic acid into a host cell can be adapted for use in the present invention. For example, the particle bombardment method of gene transfer utilizes an Accell device (gene gun) to accelerate DNA-coated microscopic gold particles into a target tissue. See, e.g., Yang et al., Mol. Med. Today 2:476-481 1996 and Davidson et al., Rev. Wound Repair Regen. 6:452-459, 2000. As another example, cell electropermeabilization (also termed cell electroporation) may be employed to deliver urease nucleic acids into cells. See, e.g., Preat, V., Ann. Pharm. Fr. 59:239-244 2001.

Synthetic gene transfer molecules can be designed to form multimolecular aggregates with plasmid DNA. These aggregates can be designed to bind to a target cell surface in a manner that triggers endocytosis and endosomal membrane disruption. For example, polymeric DNA-binding cations (including polylysine, protamine, and cationized albumin) are linked to cell-specific targeting ligands that trigger receptor-mediated endocytosis into the desired cell. See, e.g., Guy et al., Mol. Biotechnol. 3:237-248, 1995 and Garnett, M. C., Crit. Rev. Ther. Drug Carrier Syst. 16:147-207, 1999. Cationic amphiphiles, including lipopolyamines and cationic lipids, may be used to provide receptor-independent urease nucleic acid transfer into target cells. In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Forminaya et al., J. Gene Med. 2:455-464, 2000).

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Methods involving ultrasound contrast agent delivery vehicles may be used in the invention. See, e.g., Newman et al., Echocardiography 18:339-347, 2001 and Lewin et al. Invest. Radiol. 36:9-14, 2001. Gene-bearing microbubbles, which cavitate upon exposure to ultrasound, might be used to deliver the gene to a specific target tissue.

A natural or synthetic matrix that provides support for the delivered agent prior to delivery might be used in the invention. See, for example, the techniques described in Murphy and Mooney, J. Period Res., 34:413-9, 1999 and Vercruysse and Prestwich, Crit. Rev. Ther. Drug Carrier Syst., 15:513-55, 1998. Matrices suitable for use in the invention may be formed from both natural or synthetic materials and may be designed to allow for sustained release of the therapeutic agent and growth factors over prolonged periods of time. For implantation into an animal subject, a preferred matrix is resorbable and/or biocompatible (i.e., does not produce an adverse or allergic reaction when administered to the recipient host). In some embodiments of the invention, matrices are impregnated with growth factors capable of stimulating the chemotaxis and mobilization of stem cells.

DNA microencapsulation may be used to facilitate delivery of a urease nucleic acid, for example. Microencapsulated gene delivery vehicles may be constructed from low viscosity polymer solutions that are forced to phase invert into fragmented spherical polymer particles when added to appropriate nonsolvents. Methods involving microparticles are discussed in Hsu et al., J. Drug Target 7:313-323, 1999 and Capan et al., Pharm. Res. 16:509-513, 1999.

Methods involving microencapsulated recombinant cells may be used in the invention. Such an approach may be used in either in vivo or ex vivo techniques. Cells that contain an expression vector coding for urease or that have been engineered to stably express urease may be encapsulated in microcapsules that provide protection from immune mediators. Preferred microencapsulation particles, also referred to as encapsulation devices, consist of biocompatible and biodegradable components. Techniques involving microencapsulated cells are discussed in Ross et al. Hum. Gen. Ther. 11:2117-2127, 2000 and Fong et al., Crit. Rev. Ther. Drug Carrier Syst. 17:1-60, 2000.

Duration of Ammonia Producing Enzyme Expression

Urease nucleic acids of the present invention may be expressed for any suitable length of time within the host cell, including transient expression and stable, long-term expression. In a preferred embodiment, the urease nucleic acid will be expressed in therapeutic amounts for a suitable and defined length of time. Episomally replicating vectors may be used to achieve transient expression, while vectors that integrate chromosomally may be used to achieve long-term expression of a urease nucleic acid.

Effective Doses

A therapeutically effective amount is an amount which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently.

In a preferred embodiment, a dose of recombinant ammonia producing cells is about 1×10⁴ bacteria per mg wet weight; preferably, a dose of recombinant ammonia producing cells is about 5×10⁴ bacteria per mg wet weight; more preferably, a dose of recombinant ammonia producing cells is about 1×10⁵ bacteria per mg wet weight; more preferably, a dose of recombinant ammonia producing cells is about 5×10⁵ bacteria per mg wet weight; more preferably, a dose of recombinant ammonia producing cells is about 1×10⁶ bacteria per mg wet weight; more preferably, a dose of recombinant ammonia producing cells is about 5×10⁶ bacteria per mg wet weight; more preferably, a dose of recombinant ammonia producing cells is about 1×10⁷ bacteria per mg wet weight; more preferably, a dose of recombinant ammonia producing cells is about 5×10⁷, 1×10⁸ up to 5×10⁸ bacteria per mg wet weight.

In another preferred embodiment, recombinant ammonia-producing cells raise the acidic pH of the oral cavity in a patient suffering from or susceptible to dental disease to about pH 4; more preferably, the pH is raised to about pH 5; more preferably, the pH is raised to about 6.0 up to about pH 8. The dose of recombinant ammonia-producing cells and pH level can be changed according to the degree of demineralization of surface enamel as determined by a practitioner.

In another preferred embodiment, patients suffering from or susceptible to dental caries receive a thorough oral prophylaxis and a suspension of about 1×10⁶ up to 2×10⁸ recombinant host cells; preferably, 1×10⁷ recombinant cells are administered to a patient. A preferred method of administering the compositions of the invention to a patient suffering from or susceptible to dental caries (plaque) are by a cotton swab or other dental devices routinely used by a dental practitioner.

In another preferred embodiment, the compositions are administered to a patient at least once however, they can be administered on a weekly and/or monthly basis. It is within the scope of ordinary skill in the art for a dental practitioner to determine if a patient needs further doses. It may be necessary to administer the compositions every 6 to 12 months and can be part of a routine oral hygiene regimen every 6 months as recommended by the American Dental Association. The compositions can be administered as a prophylaxis for patients susceptible to dental caries. Optionally, the administration of the compositions can be determined on a case-by-case basis by a dental practitioner. In accordance with the invention, one dose of the compositions disclosed herein, could last the lifetime of a patient.

Toxicity and therapeutic efficacy of the compositions of the invention can be determined by standard pharmaceutical procedures, using cells in culture and/or experimental animals to determine the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. While compositions that exhibit toxic side effects may be used, care should be taken to design a delivery system that targets such compounds to the site of the infection or tissues to be treated in order to minimize potential damage to uninvolved tissue and thereby reduce side effects.

The data obtained from cell culture assays and animal studies can be used in formulating a range of dosage for use in humans. The dosage of such compounds lies preferably within the amount of ammonia produced by the cells to increase the pH of the environment from about pH 4 up to pH 9 that include an ED₅₀ with little or no toxicity. The dosage may vary within this range depending on the dosage form employed and the route of administration utilized.

Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

The following examples serve to illustrate the invention without limiting it thereby. It will be understood that variations and modifications can be made without departing from the spirit and scope of the invention.

### Example 1

### Identification and Characterization of the Nickel Uptake System in the Urease Gene Cluster of Streptococcus salivarius 57.I

This example describes the isolation of an exemplary bacterial nickel transporter system useful in the practice of the invention.

Materials and Methods:

Bacterial strains, growth conditions, and reagents. S. salivarius 57.I and its UreMQO-deficient derivative were routinely grown in brain heart infusion (BHI, Difco Laboratories) at 37° C., in a 5% CO₂ atmosphere. Recombinant E. coli strains were routinely maintained in L broth. Kanamycin (Km) was included in the growth medium, when indicated, at 750 μg ml⁻¹ or 50 μg ml⁻¹ for recombinant S. salivarius or E. coli strains, respectively. All chemical reagents and antibiotics were purchased from Sigma. ⁶³NiCl₂ (9.87 mCi Ni mg⁻¹) was purchased from Amersham Biosciences (Piscataway, N.J.). To obtain cultures grown under neutral or acidic conditions, cells were grown in BHI comprising 50 mM potassium phosphate buffer, pH 7.4 (BHI-KPB), BHI, or BHI that had been adjusted to pH 5.8 by the addition of 2 N HCl (BHI-HCl).

Nucleic acid manipulations. Genomic DNA from S. salivarius 57.I was isolated as previously described (Chen et al., 1996). Plasmid DNA from recombinant E. coli strains was purified by using the QIAprep Spin Plasmid kit (Qiagen, Inc., Valencia, Calif.). Cloning, Southern blot analysis and hybridizations were carried out by using an established protocol (Ausubel et al., 1989). Restriction endonucleases, DNA polymerases, and RNA reverse transcriptase (RT) were obtained from Invitrogen (Carlsbad, Calif.) or New England Biolabs (Beverly, Mass.). Total cellular RNA from S. salivarius strains was isolated as described elsewhere (Chen et al., 1998). Levels of ure-specific mRNA were quantitated by densitometry using slot blot analysis, and the hybridization and washes were carried out at high stringency as described previously (Chen and Byrne, 1996).

Isolation of ureM, Q and O. The region immediately 3′ to the ure cluster was amplified from S. salivarius 57.I by PCR using primers derived from the Streptococcus thermophilus LMG18311 genome sequences.

The primers used for completing Streptococcus salivarius 57.I ure operon via PCR were as follows:

1. Product #1:

(SEQ ID NO:5)

| | |
|---|---|
| S4420: | GCTTATCGGCTTGGTAGAAG |

(SEQ ID NO:6)

| | |
|---|---|
| TherAS17220: | GCTTCGTGCTTCAATCC |
| 2. Product #2: | |

(SEQ ID NO:7)

| | |
|---|---|
| 55290S: | GTTCTGCTCCAGTTAGATATCAC |

(SEQ ID NO:8)

| | |
|---|---|
| TherAS17750: | GCAATCTCATCATAGACAG |

3. Product #3

(SEQ ID NO:9)

| | |
|---|---|
| TherS17630: | CTCAAAGAATGGCTGAAGG |

(SEQ ID NO:10)

| | |
|---|---|
| TherAS18180: | ACCTAGAACGATTTCCTACT |

PCR was initiated with 5 cycles at a less stringent annealing temperature (50° C.), followed by 20 cycles at a more stringent annealing temperature (55° C.). All PCR products were cloned onto plasmid PCR^{a}II (Invitrogen) and the sequences were determined.

Construction of a UreMQO-deficient S. salivarius. A HindIII-NcoI fragment, 2.9 kbp in size, comprising the 3′ portion of ureG, ureD through ureM, and the 5′ portion of ureQ (FIG. 1) was initially subcloned from pMC11 (Chen et al., 1996) onto pGEM3Zf(+) to generate plasmid pMC281. A DNA fragment comprising a Km resistance marker flanked by transcription/translation terminators (Ωkan) (Perez-Casal et al., 1991) was subsequently cloned onto the unique NsiI site, located at the beginning of ureM, on plasmid pMC281. The resulting chimeric plasmid, pMC282, was transferred into strain 57.I by electroporation (Chen et al., 1998) to introduce a polar mutation in ureM by allelic exchange. The configuration of the double-crossover integration of the Km resistance marker was confirmed by PCR and Southern blot analysis.

Nickel accumulation. A nickel accumulation assay was adopted from Wolfram et al. (1995) with minor modifications. Briefly, overnight cultures of S. salivarius strains in BHI were diluted 1:50 into fresh BHI-KPB or BHI comprising 500 nM ⁶³NiCl₂. Unlabeled metal chlorides as competitors were added to a final concentration of 5 μM. All cultures were grown at 37° C. for 5 hours at which point the O.D.₆₀₀ of the cultures was approximately 0.9. Cells were harvested, washed twice with an equal volume of ice-cold buffer A (50 mM Tris-HCl, 10 mM MgCl₂, pH 7.5) and then concentrated 10-fold in the same buffer. The radioactivity of an aliquot of the cell suspension (100 μl) was determined by liquid scintillation counting. To monitor the amount of ⁶³Ni⁺² accumulated intracellularly over time, cells were grown in BHI at 37° C. for 3 hours and 45 minutes prior to addition of ⁶³NiCl₂. A 1-ml aliquot of the cell suspension was harvested every 15 minutes post-addition and processed as described above. The CFU in the same amount of cell suspension was determined by serial dilution and plating on BHI agar plates. Alternatively, the same amount of cell suspension was subjected to mechanical disruption in the presence of glass beads (0.1 mm diameter) by homogenization in a Bead Beater (Biospec Products) for a total of 40 s at 4° C. The concentration of each cell lysate was determined by using the Bio-Rad Protein Assay based on the method of Bradford (1976). The cellular content of ⁶³NiCl₂ was expressed as pmol (10⁹ CFU)⁻¹ or pmol (mg protein)⁻¹.

Urease assays. Overnight cultures of the wild-type S. salivarius 57.I and its UreMQO-deficient derivative were diluted 1:20 into fresh BHI-KPB, BHI, or BHI-HCl comprising 0, 2.5, 5, 10, 25, 50, 75, or 100 μM NiCl₂, respectively, and grown to O.D.₆₀₀ 0.65. Cultures were harvested by centrifugation, washed once with an equal volume of 10 mM KPB, pH 7.0, and then concentrated 40-fold in the same buffer. Concentrated cell suspensions were subjected to mechanical disruption in the presence of an equal volume of glass beads (0.1 mm diameter) by homogenization in a Bead Beater™ (Biospec Products, Bartlesville, Okla.) for a total of 40 seconds at 4° C. The concentration of each cell lysate was determined by using the Bio-Rad Protein Assay based on the method of Bradford (1976). Urease activity was measured as described previously (Chen et al., 1996) and normalized to protein concentration.

Preparation of an anti-UreC antiserum. To obtain an anti-UreC antiserum, the complete structural gene encoding the α subunit of S. salivarius 57.I urease was amplified by PCR using a primer pair:

(SEQ ID NO:11)

ureCBglIIS: 5′-TAGAAAGAGGACAGATCTATGAGTT-3′
and

(SEQ ID NO:12)

ureCSalIAS: 3′-CTCATTTATATAGGTCGACCCTTAGA.

For cloning purposes, a BglII restriction site and a SalI site (underlined) was included in ureCBglIIS and ureCSalIAS, respectively. The PCR product was subsequently cloned into BamHI- and SalI-digested pQE30 (Qiagen) to generate a transcriptional fusion of six consecutive histidine residues to the N-terminus of UreC. The organization of the chimeric plasmid was confirmed by sequence analysis. The synthesis of the recombinant UreC protein was induced by the addition of isopropyl-β-D-thiogalactoside to a final concentration of 2 mM. The cultures were harvested 5 hours post induction and concentrated to 1/40 of the original volume in 10 mM Tris, pH 8.0. The concentrated cell suspensions were subjected to mechanical disruption as described above. The recombinant UreC protein was initially purified by nickel affinity chromatography. The N-terminal amino acid sequence of the recombinant UreC was obtained by using Edman degradation on fractions comprising peak amounts of the recombinant product, and the sequence was found to be Met-Arg-Gly-Ser-(His)₆-Met-Ser-Phe-Lys-Met-Asp. The four amino acids N-terminal to the His-tag are derived from the vector (pQE30) and the sequence C-terminal to the His-tag is from UreC.

The fractions were further purified by SDS-PAGE and a portion of the gel comprising the 61.8-kDa UreC was excised and used for antiserum production in rabbits (Lampire Biological Laboratories, Pipersville, Pa.). The antiserum was further adsorbed with total cell lysates of E. coli M15 [pREP4] harboring pQE30 and of a non-ureolytic streptococcal species, S. mutans UA159, prior to its use in Western blot analysis, because rabbits often harbor antibody that reacts with streptococcal components.

Results:

Isolation and sequence analysis of ureM, Q and O. Urease biogenesis by bacteria requires a high affinity Ni²⁺ uptake system. By searching the partial genome sequence of a closely related organism, S. thermophilus LMG18311, 3 ORFs homologous to those encoding proteins similar to the ABC-type cobalt transport system found in cobalamin biosynthesis operons (cbiMQO) were identified immediately 3′ to the ure operon. To determine whether these genes were present in S. salivarius, a chromosomal walking approach was employed to obtain sequence 3′ to ureD by PCR from S. salivarius using primers homologous to the cbiM, Q, and O genes of S. thermophilus. Approximately 3 kbp immediately 3′ to ureD was obtained from the 3 overlapping PCR products.

FIG. 1 shows a schematic diagram of the ure operon of S. salivarius 57.I. A restriction endonuclease map of the chromosome comprising the ure cluster is shown on the top line. The relative location and size of each ORF are indicated. The directions of transcription of the ure operon and flanking genes are indicated by horizontal arrows. The limits of the partial ure cluster in S. mutans ACUS6 are indicated by vertical arrows. The locations and orientations of primer pairs used in RT-PCR are indicated by horizontal arrows immediately under the restriction map. Below, the locations of the PCR products are shown in solid lines.

Referring to FIG. 1, three complete ORFs and an additional partial ORF, all in the same orientation as the ure operon, could be identified within this 3-kbp region. ORF1, with its RBS embedded in ureD, is located 3 bases 3′ to the stop codon of ureD. ORF2 overlaps with ORF1 by 2 bases and ORF3 is located 1 base 3′ to ORF2. The partial ORF is located 165 bp 3′ to ORF3.

To determine whether these ORFs were co-transcribed with the ure operon, RT-PCR was employed to detect the existence of contiguous transcripts between ureD, ORF1, 2 and 3. PCR products were generated from RT-PCR. 1% of total cDNA generated by RT-PCR from each RNA sample was amplified with specific primers shown schematically in FIG. 1, and 10% of the total PCR products were run on a 1.2% Tris-acetate-EDTA gel. PCR products were generated with a primer pairs specific for 1) the ureDM intergenic region; 2) the ureMQ intergenic region and 3) the ureQO intergenic region. The primer pairs used for detecting mRNA within S. salivarius 57.I ure operon via RT-PCR had the following sequences:

1. between ureD and ureM:

(SEQ ID NO:13)

| | |
|---|---|
| S3660: | CTGAATTTAGAGTCTGATTTTGC |

(SEQ ID NO:14)

| | |
|---|---|
| AS4175: | GGCATTCGCACCAAAGGC |

2. between ureM and ureQ:

(SEQ ID NO:15)

| | |
|---|---|
| S4420: | GCTTATCGGCTTGGTAGAAG |

(SEQ ID NO:16)

| | |
|---|---|
| AS5045: | GGCTACAATCCACAAAACTGTG |

3. between ureQ and ureO:

(SEQ ID NO:17)

| | |
|---|---|
| S5290: | GTTCTGCTCCAGTTAGATATCAC |

(SEQ ID NO:18)

| | |
|---|---|
| AS17750: | GCAATCTCATCATAGACAG |

Control reactions without RT were carried out identically to the experimental samples, but without RT. Products were generated from cells grown in BHI-KPO₄, BHI, and BHI-HCl. The results indicated the presence of contiguous transcript(s) between ureD, ORF1, 2, and 3, suggesting that all three ORFs could be co-transcribed with the urease genes and were part of the ure operon.

A stable stem-loop structure, with ΔG^{O}=−10.3 kcal, followed by a stretch of 6 T residues, which could potentially function as a rho-independent terminator, was identified 7 bases 3′ to the stop codon of ORF3, and no transcript could be detected by RT-PCR between ORF3 and the partial ORF, indicating that this partial ORF is not part of the ure operon. In addition, the sequence of this partial ORF encoded a truncated peptide that shared significant homology (60% to 70% identity) with the N-terminus of the substrate-binding proteins of amino acid ABC-type transporters (AtmA) from other streptococcal species.

The lack of involvement of this ORF in urea metabolism was further confirmed by using a recombinant strain in which this ORF was insertionally inactivated. No effect on urease activity could be detected when this ORF was inactivated, further indicating that this ORF was not part of ure operon. ORF1, 2, and 3 were designated as ureM, Q, and O, respectively, and it was concluded that the ure operon of S. salivarius consists of 11 genes (ureIABCEFGDMQO). The complete sequence of the ure operon has been deposited with GenBank and assigned accession number U35248, and is described herein as SEQ ID NO: 1.

Translation of S. salivarius ureM predicted a protein of 325 aa with pI=8.99, corresponding to a calculated mass of 35.2 kDa. UreM shared significant degrees of homology with CbiM, encoding an integral membrane protein involving in cobalt transport for cobalamin biosynthesis, from Thermoanaerobacter tengcongensis (47% identity and 64% similarity) and Clostridium acetobutylicum (39% identity and 58% similarity). The conserved domain of the permease component of ABC-type Co²⁺ transport systems could also be identified within UreM. S. salivarius ureQ, 777 nt, encoded a protein of 19.1 kDa with pI=9.63. The highest degree of homology was observed between ureM and genes encoding cobalt permeases from Lactobacillus plantarum (28% identity and 43% similarity) and from C. acetobutylicum (24% identity and 46% similarity). S. salivarius ureO, 714 nt, encoded a protein of 27.6 kDa and pI=5.44. UreO shared 44%, 40% and 37% to the ATPase components of ABC-type cobalt transport systems (CbiO) of L. plantarum, C. acetobutylicum and T. tengcongensis, respectively. Significant levels of homology to the ATP-binding proteins of cobalt ABC transporters from Methanosarcina species were also observed (35% identity). The linker peptide and the Walker A and Walker B motifs commonly found in a number of ATP- and GTP-binding and hydrolyzing proteins were also observed in the perspective positions in the protein.

Functional analysis of UreMQO in Ni²⁺ accumulation. To examine the impact of UreMQO in urease biosynthesis, a polar mutation was introduced into ureM, 4 bases 3′ to the ATG. The ability to accumulate ⁶³Ni²⁺ was examined in S. salivarius wild-type and UreMQO-deficient strains. Earlier studies demonstrated that urease expression in S. salivarius is predominately regulated by growth pH (Chen et al., 1996, 1998). At neutral pH, expression is almost completely repressed. Induction occurs and increases as the growth pH becomes more acidic. Therefore, to determine whether the capacity to accumulate Ni⁺² in both the wild-type and UreMQO-deficient strains was also regulated by pH, cells were cultured in BHI-KPO₄, in BHI alone, or in BHI-HCl, as detailed in the Materials and Methods, to mid-exponential phase, at which point the cultures were at approximately pH 7, 6, and 5, respectively.

When cells were incubated with ⁶³NiCl₂ at a concentration of 500 nM, it was found that, parallel to total urease activity in cells grown at different pH values, the wild-type strain accumulated ⁶³Ni²⁺ in a pH-dependent manner (Table 1).

TABLE 1
Nickel accumulation in S. salivarius 57.l and UreMQO-deficient strain.

| | cpm × CRU⁻¹ × 10⁻⁸ | | |
|---|---|---|---|
| Strain | BHI—KPO₄ | BH | BHI—HCl |
| S. salivarius 57.I | 19.5 ± 1.7 | 327.0 ± 81.4 | 1105.3 ± 152.4 |
| S. salivarius UreM⁻ | 2.4 ± 0.9 | 22.7 ± 1.0 | 49.6 ± 9.5 |

The maximum level of accumulation was observed with cells grown in BI-1-HCl, with approximately 3-fold and 50-fold increases compared to cells grown in BHI and BHI-KPO₄, respectively. In contrast, ⁶³Ni²⁺ accumulation in the UreMQO-deficient strain was virtually abolished, indicating that UreMQO are essential for the uptake of ⁶³Ni²⁺ from the environment.

The accumulation of ⁶³Ni²⁺ in wild-type S. salivarius during growth was further confirmed by incubating the cells with different amounts of ⁶³Ni²⁺. It was found that the amount of intracellular ⁶³Ni²⁺ was a direct result of the concentrations of exogenous of ⁶³Ni²⁺. The substrate specificity of UreMQO in wild-type S. salivarius was examined by addition of unlabeled Co⁺², Zn⁺², Mn⁺², Mg⁺² and Ni⁺² at a 10-fold excess (5) in growth medium comprising 500 nM ⁶³Ni²⁺. Results showed that excess amounts of unlabeled Co⁺², Zn⁺², Mn⁺² or Mg⁺² chloride did not result in any significant decrease in the amount of ⁶³Ni⁺² accumulated. However, inclusion of 5 μM unlabeled NiCl₂ in the growth medium inhibited the accumulation of ⁶³Ni⁺² by 85%, indicating that UreMQO have specificity for Ni⁺² ion.

A modified accumulation assay was used in a time-course study, in which a larger volume of cell suspension was collected by centrifugation at each time point, followed by two-washes with buffer comprising 10 mM Mg⁺² to remove non-specifically bound ⁶³Ni⁺². It was found that the expression of the S. salivarius urease operon, which is tightly regulated by growth pH 7, was repressed until the pH of the culture reached about 6.5, at which time urease specific activity increased linearly and peaked during the late exponential phase of growth. To ensure that nickel accumulation was monitored during biogenesis of an active urease, ⁶³Ni⁺² was added to the cultures at 3 hours and 45 minutes post inoculation and the amount of ⁶³Ni⁺² was monitored every 15 minutes afterwards. A time-dependent accumulation was observed and the accumulation reached maximal levels 60 minutes post-addition of ⁶³Ni⁺². No significant uptake of nickel occurred prior to the induction of the urease operon.

Urease activity was enhanced by exogenous NiCl₂. It is known that the average concentration of nickel in the natural environment is in the nM range and the most commonly occurring oxidation state of nickel is Ni(II). However, when Ni²⁺ is present at higher concentrations, it can be transported by Mg²⁺ transport systems (Kehres et al., 1998; Smith and McGuire 1998).

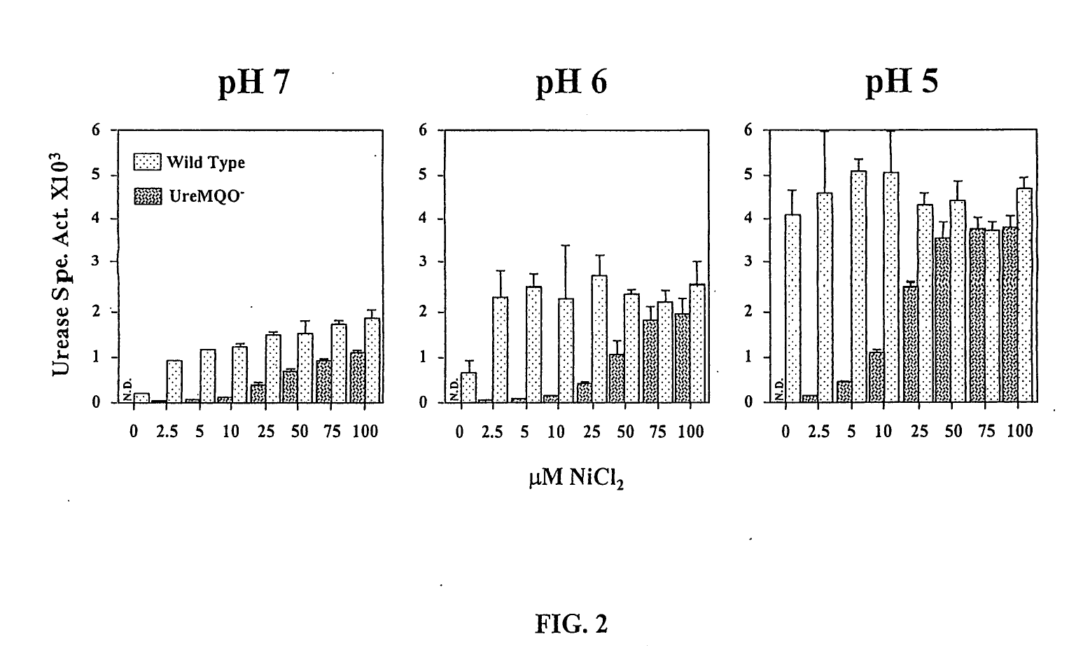

To determine the influence of UreMQO in overall urease biosynthesis and whether high concentrations of exogenous Ni²⁺ could compensate for the deficiency of the Ni²⁺-specific uptake system, most likely through a Mg²⁺ uptake system, urease activities were examined in wild-type and UreMQO-deficient strains at different growth pH, with or without additional NiCl₂ (FIG. 2). No detectable urease activity was observed in the UreMQO-deficient strain in the absence of supplemented NiCl₂, regardless of the growth pH. When cells were grown at neutral pH, supplementation with as little as 2.5 μM NiCl₂ was able to partially restore the ureolytic phenotype in the UreMQO-deficient strain, and the levels of urease activity increased in a NiCl₂ concentration-dependent manner. NiCl₂-dependent increases in urease activity were also observed in cells grown in BHI and BHI-HCl, and the highest levels of urease activity at each NiCl₂ concentration supplied to the system were consistently observed in cells grown in BHI-HCl. In the absence of exogenous NiCl₂, urease activity in the wild-type strain was solely regulated by growth pH, with the highest urease activity observed in cells grown in BHI-HCl.

When cells were grown at neutral pH (BHI-KPO₄), urease activity in the wild-type cells increased in response to increasing concentrations of NiCl₂, and reached a maximum level at 25 μM NiC₂. The enhancement of urease activity by NiCl₂ was less prominent when cells were grown at acidic pH. When cells were grown in BHI without any buffer, the highest level of activation by NiCl₂ occurred at 2.5 μM, but there was no significant increase in urease activity with additional NiCl₂ when cells were grown in BHI-HCl. Therefore, under conditions where the ureMQO may not be fully induced, addition of nickel can increase the amount of activated urease, suggesting that nickel-uptake is a limiting factor in urease biogenesis at neutral pH. Under acidic conditions, when expression of the operon is optimal, the accumulation of intracellular Ni⁺² via the activity of UreMQO appears to be sufficient for the assembly of all translated urease subunits and additional nickel had no impact on the levels of urease activity. Utilizing the capacity of nickel accumulation as a limiting factor for total urease activity at neutral pH provides an additional level of control for urease expression in S. salivarius.

The expression of urease genes and urease activity in H. pylori is regulated by the availability of nickel (van Kleit et al., 2001). Supplementation of nickel at μM concentrations in the growth medium leads to higher levels of transcription of the operon. On the other hand, the differential expression of urease in S. salivarius 57.I in response to environmental pH is predominately regulated at the transcriptional level from a promoter 5′ to ureI (p_{ureI}) (Chen et al., 1998). To investigate whether the up-regulation of urease activity by NiCl₂ in S. salivarius could be mediated at the level of urease gene transcription from P_{ureI}, the level of expression was monitored in a recombinant S. salivarius strain PureIcat, which carries a single copy of P_{ureI}-cat fusion at the lacZ locus (Chen et al., 2002), by measuring CAT specific activity. The results showed that no significant differences in CAT activity were observed in cells grown in different concentrations of NiCl₂, regardless of the growth pH, confirming that the higher level of urease activity in the presence of NiCl₂ did not result from elevated levels of ure transcription but rather was regulated at the level of enzyme activation.

To further confirm that the higher level of urease activity observed in the presence of exogenous NiCl₂ in S. salivarius was not due to increased levels of the urease message, total cellular RNA was isolated from wild-type cells and the UreMQO-deficient strain grown in BHI-KPO₄ that were supplemented with 0 to 100 μM NiCl₂, and the amounts of ureC- and ureM-specific mRNA were quantitated by slot-blot analysis. The results showed that no significant differences in the levels of ureC-specific mRNA could be detected in the wild-type strain under all concentrations of NiCl₂ tested. Similarly, no differences in ureC-specific mRNA were detected between the wild-type and the UreMQO-deficient strains in response to nickel, indicating that the nickel-responsive activation of urease activity is not mediated at the transcriptional level. As expected, the levels of ureM-specific message in the wild-type strain were not influenced by the amount of NiCl₂ in the growth medium and there was no detectable ureM signal in the UreMQO-deficient strain.

The levels of UreC protein (61.8 kDa), the α subunit of the urease, were also examined by Western blot analysis using an anti-UreC polyclonal antibody in strains grown in BHI-KPO₄, BHI or BHI-HCl comprising different concentrations of NiCl₂. Of note, a non-specific signal, approximately 58 kDa, was consistently observed in both wild-type and UreMQO-deficient strains, in spite of extensive absorption with both E. coli and S. mutans total cell lysates. No significant differences in the levels of UreC could be detected between the wild-type and UreMQO-deficient strain under the same growth conditions without additional NiCl₂, indicating that the absence of detectable urease activity in the UreMQO-deficient strains in the absence of exogenous NiCl₂ was due to the inability to transport NiCl₂ in the mutant, rather than the lack of production of urease subunits. The levels of UreC protein in both the wild-type and UreMQO-deficient strains were not influenced by the amount of NiCl₂. Thus, the higher levels of urease activity observed in the wild-type cells at neutral pH (BHI-KPO₄) in the presence of exogenous NiCl₂ at μM concentrations likely resulted from the non-specific transport of NiCl₂, presumably via Mg⁺² permease(s).

### Example 2

### Construction of Recombinant Bacteria Expressing Urease Enzymes

For construction of urease producing plaque bacteria, integration vectors are first selected that allow for stable establishment of foreign genes in the appropriate bacterial strain, without interference with normal functions such as growth and colonization. For use in S. mutans and some related organisms that harbor the gtfA gene, an integration vector, designated pBGK, has been described (Wen and Burne, 2001). Studies using this vector have shown that insertional inactivation of gtfA does not affect colonization of the bacteria.

For insertion of genes into S. gordonii, at the gtfG locus, a vector designated pMJB8 can be used. A schematic diagram of an embodiment of a gtfG integrating vector, designated pMJB8, is shown in FIG. 9.

Alternatively, vectors can be used that allow insertion into the mannitol operon of S. mutans. Mannitol is a non-cariogenic sugar that is not abundant in the diet. A schematic diagram of an embodiment of an insertion vector of this type, designated pMC321, is presented in FIG. 10.

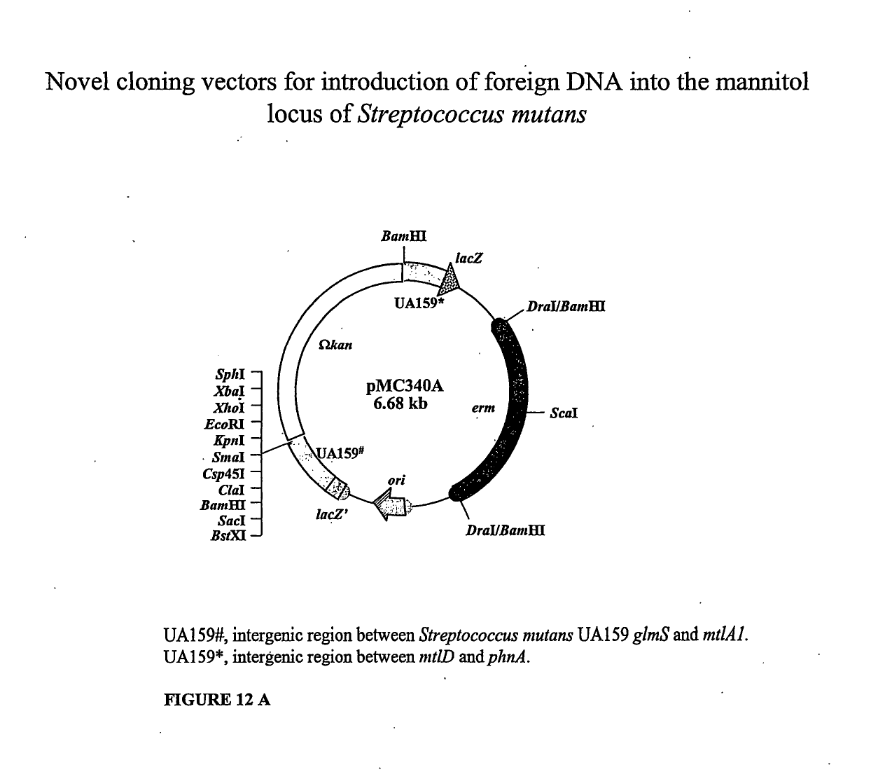

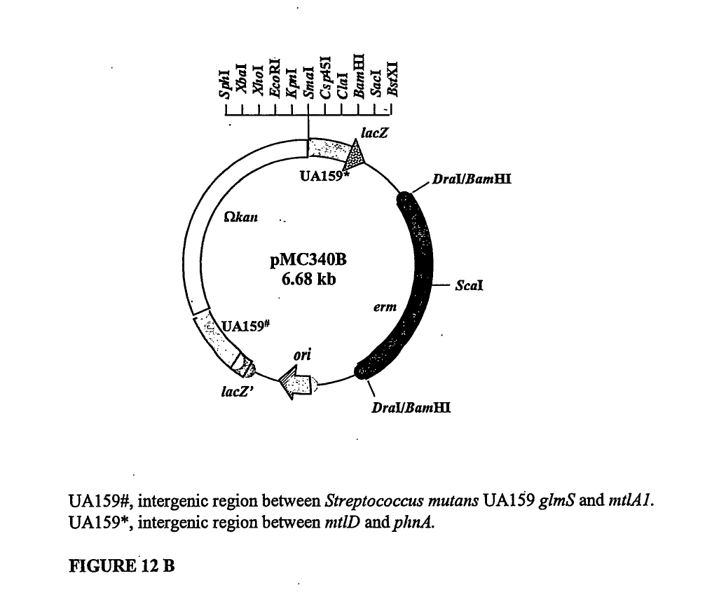

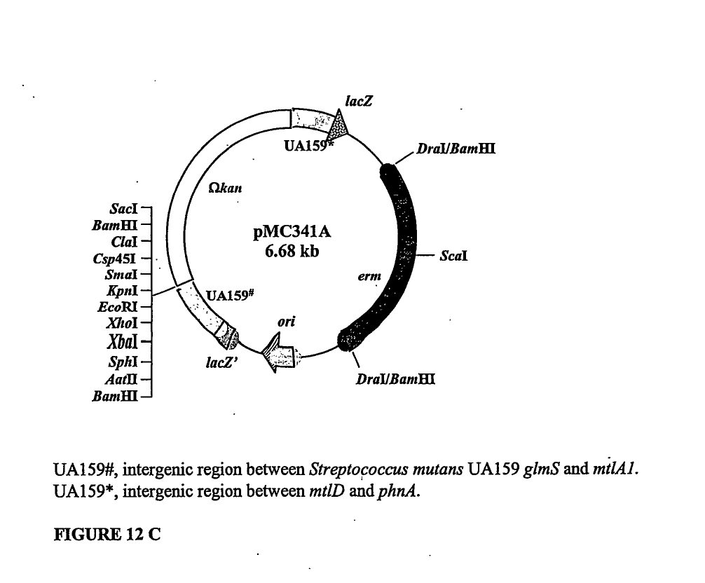

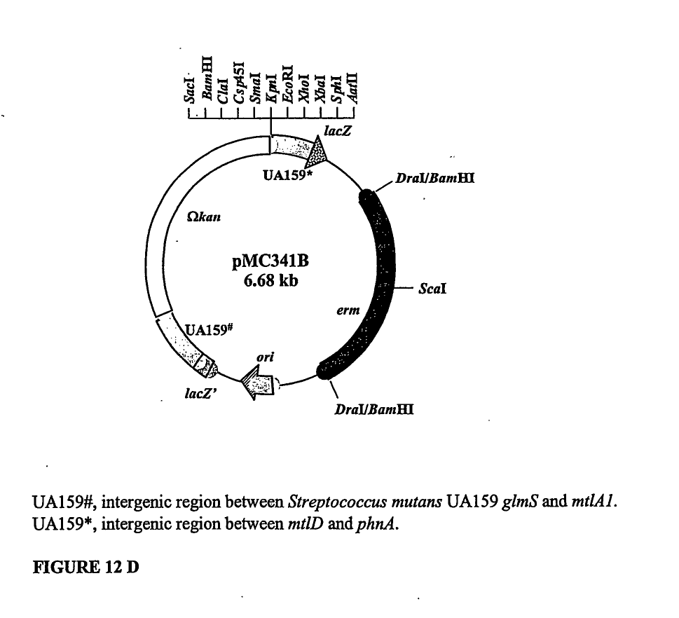

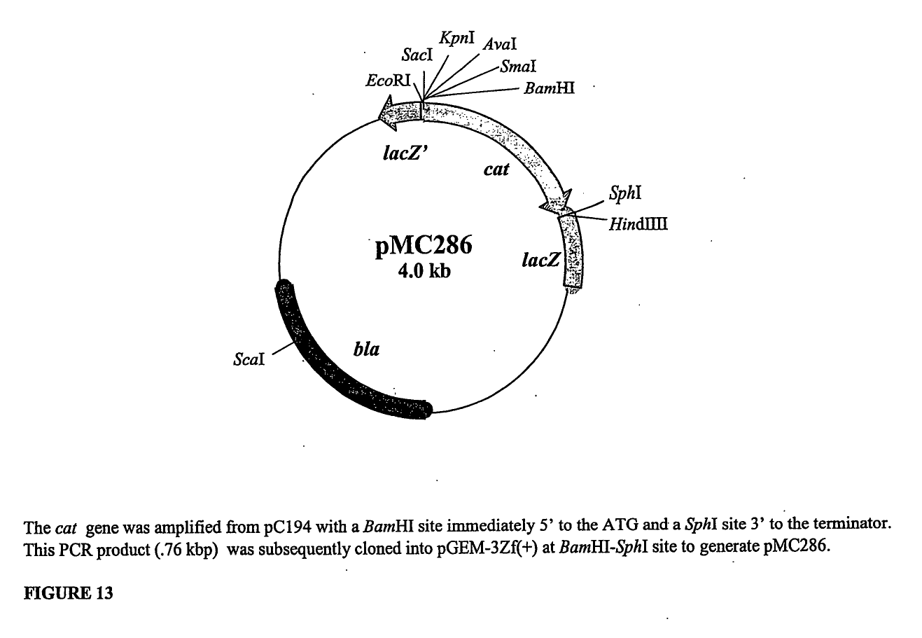

Construction of alkali-generating plaque streptococci: Development of stable, ammonia-producing plaque streptococci has been achieved with the design and engineering of new strains that produce high levels of urease in the absence of exogenous nickel. We have engineered new integration vectors for use in Streptococcus mutans. In this case, the mannitol operon of S. mutans UA159 was targeted as the insertion sight. See for example, FIGS. 12A-12D and FIG. 13. Mannitol is only poorly cariogenic and in most cases is not a major constituent of the human diet. It is rather poorly metabolized by S. mutans, so loss of mannitol utilization capabilities should not affect competitive fitness of the organism in vivo. The target gene cluster is annotated as smu1082 in the Oral Pathogens database (http://www.stdgen.lanl.gov/oragen/).

The insertion vector allows for a double-crossover recombination into the mtl gene cluster so that genes can be integrated into the chromosome in single copy and no selective pressure is needed to maintain the genes in culture medium or, presumably, in a mammalian host. The genes can be tagged with a variety of antibiotic resistance determinants to track integration and the organisms acquire a phenotype where they are unable to grow on mannitol as the sole carbohydrate source.

Secondly, we have spliced together an entire, intact urease gene cluster, including ureIABCEFDGMQO, driven off of the intact urease cognate promoter. This was not at all trivial because of toxicities associated with expressing ureI in E. coli

Third, we were able to integrate an entire, functional urease gene cluster into S. mutans at the mtl locus in stable form. These strains expressed a functional urease in the absence of exogenous nickel, as did E. coli carrying the ureI-O genes.

To overcome any problems with low level expression, we engineered a strong promoter from a tetracycline resistance cassette to drive expression of the entire operon. This new strain of S. mutans produces tremendously high levels of the urease enzyme, substantially more than the highly ureolytic Streptococcus salivarius 57.I from which the urease genes were derived. No supplementation with nickel is necessary for high activity.

These strains are to be used for testing in an appropriate animal model. We have also designed modified strains that can be engineered for testing in humans if the animal experiments are successful. These include not only strains of S. mutans, but also more benign commensal plaque streptococci that can be used as replacement strains.

Other vectors that allow for the stable establishment of foreign genes can also be used, their designs being based on non-essential genes, for example of S. gordonii or S. sanguis.

For laboratory analyses, antibiotic resistance markers are preferably utilized to tag the insertion vectors so that transformants carrying the foreign genes can be selected on plates comprising antibiotics. Vectors useful for human applications are essentially identical to those useful for in vitro studies, except for lack of antibiotic resistance markers.

The general vector design is consistent for each vector. For example, a commercially available E. coli plasmid cloning vector comprises the appropriate 5′ and 3′ fragments flanking the desired insertion site. For laboratory applications, those sequences flank an antibiotic resistance marker (generally kanamycin, tetracycline or erythromycin) and a unique cloning site(s) into which the foreign genes are cloned.

Standard cloning protocols are used to introduce the urease genes, for example ureIABCEFGDMQO from S. salivarius 57.I, driven by either the urease promoter or by a stronger heterologous promoter, into a site between the 5′ and 3′ fragments of the target site. Confirmation that the genes are correctly inserted is performed using standard methods.

The plasmid can be digested with restriction enzyme(s) and the DNA is then used to transform plaque streptococci that have been prepared by standard methods to accept DNA. Selection for antibiotic resistance, and subsequent studies to confirm that the genes are integrated and that the strains express the desired activities are performed. For strains suitable for human applications, in which antibiotic resistance is preferably omitted, the strains are screened for their capacity to produce ammonia or other alkalis from appropriate substrates. This can be done by biochemical assays or by the use of differential media that detect end products or pH changes in the media.

### Example 3

### Isolation of the Gene Cluster for the Arginine Deiminase System from Streptococcus gordonii DL1

This example describes the isolation of an exemplary bacterial arginine deiminase system useful in the practice of the invention.

Materials and Methods:

Bacterial strains, growth conditions, and reagents. S. gordonii DL1 was maintained in brain heart infusion broth (BHI, Difco Laboratories, Detroit, Mich.) at 37° C., in a 5% CO₂ atmosphere. S. gordonii ArcR⁻ was selected and maintained on BHI agar supplemented with 250 μg ml⁻¹ kanamycin (Km). Recombinant E. coli strains were routinely maintained on L agar supplemented, when indicated, with 25 μg ml⁻¹ chloramphenicol (CM) or 50 μg ml⁻¹ Km. All chemical reagents were obtained from Sigma.

DNA manipulations and construction of a subgenomic DNA library of S. gordonii DL1. Genomic DNA from S. gordonii DL1 was isolated as previously described (Chen et al., 1996). Plasmid DNA was isolated from E. coli as described (Birnboim and Doly, 1979). Plasmid DNA to be used in sequencing reactions was prepared from E. coli DH10B using the QIAprep Spin Plasmid Kit (Qiagen Inc., Valencia, Calif.). Cloning, electrophoretic analysis of DNA fragments, Southern blot analysis and hybridizations were carried out using established protocols (Ausubel et al., 1989). Restriction and DNA modifying enzymes were purchased from Life Technologies (Rockville, Md.) or New England Biolabs (Beverley, Mass.).

For preparation of subgenomic DNA libraries, total chromosomal DNA of S. gordonii DL1 was digested to completion with XbaI or HincII. The digested DNA fragments were separated on agarose gels and the XbaI fragments of 6-8 kbp or HincII fragments of 3-5 kbp were enriched by gel purification using the Elu-Quik DNA Purification Kit (Schleicher & Schuell, Keene, N.H.). The isolated DNA fragments were ligated onto XbaI- or HincII-digested, phosphatase-treated pSU20 (Bartolome et al., 1991), respectively. The ligation mixtures were used to transform E. coli DH10B, and Cm resistant transformants were selected.

Development of an arcB-specific probe. An internal fragment of S. gordonii DL1 arcB, encoding cOTC, was generated by PCR using degenerate primers that were designed based on alignments of known anabolic and catabolic OTCs.

Primer 5′ arcBS: 5′-GGNGA(T/C)GCN(A/C)GNAA(T/C)AA(T/C)AT-3′ (SEQ ID NO:19), comprised the sequence encoding amino acid positions approximately 160-170 of OTC, i.e., GDARNNMGN.

Primer 3′arcBAS: 5′-TGCATNC(G/T)(A/G)TT(T/C)TCNGC(T/C)TG-3′ (SEQ ID NO:20), comprised the anti-sense sequence of that encoding amino acid positions approximately 315-325, i.e., HMRNEAQ.

Each reaction consisted of 5 cycles at a reduced stringency annealing temperature (50° C.) followed by 20 cycles at a stringent annealing temperature (55° C.). The product with the correct predicted size was gel purified prior to cloning into pCR™II to generate pJZ23. Southern blot analysis confirmed that the PCR product hybridized to S. gordonii DL1 chromosome at high stringency. Sequence analysis and Blast searches were performed to confirm that the product shared high degrees of homology with other cOTCs.

Construction of an ArcR-deficient strain of S. gordonii DL1. A 1.5-kbp fragment containing the S. gordonii arcR gene was amplified by recombinant PCR (Higuchi, 1990) to introduce a unique BamHI site 152 bp 3′ to the ATG of arcR. A BamHI fragment containing a Km resistance determinant flanked by transcription/translation termination signals Ωkan (Perez-Casal et al., 1991) was subsequently cloned into the BamHI site within the PCR product. The resulting plasmid was used to transform S. gordonii (LeBlanc, 1976) to generate an ArcR-deficient mutant via allelic exchange. The correct configuration of the integration event in Km-resistant transformants of S. gordonii was confirmed by Southern blot analysis using Ωkan- and arcR-specific probes.

RNA isolation and primer extension analysis. Wild-type S. gordonii DL1 and its ArcR-deficient derivative were grown to mid-exponential phase in TV medium (Burne, 1999) comprising 10 mM glucose or galactose, with or without 1% arginine. Total RNA was isolated from each culture as previously described (Chen et al., 1998). The arcA transcription initiation site was determined by primer extension analysis (Ausubel, 1989) using primer AsarcA: 5′-CAGGTCTATGTAACATAACTTTTTTCA-3′ (SEQ ID NO:21), which comprised the antisense sequence of arcA located 42-bases 3′ to the translational start site. For each reaction, radio-labeled primers were incubated with 50 μg of total cellular RNA at 42° C. for 90 min to allow complete annealing. Products were analyzed alongside a DNA sequencing reaction using the same primer.

Arginine deiminase assay. Arginine deiminase activity was measured by monitoring citrulline production from arginine as previously described (Archibald, 1944). Briefly, S. gordonii DL1 and the ArcR-deficient strain were grown in TV comprising 10 mM glucose or galactose, with or without 1% arginine, to O.D.₆₀₀≈0.55. Cells were harvested by centrifugation, washed once with 10 mM hexanoic acid, and resuspended in 1/10 of the original culture volume in the same buffer. The cell suspension was disrupted in a bead-beater (Biospec Products, Inc., Bartlesville, Okla.) for a total of 40 sec at 4° C. The total cell lysates were recovered by centrifugation and the concentration was measured using a protein assay based on the method of Bradford (Bio-Rad, Hercules, Calif.). Bovine serum albumin served as the standard.

Results:

Isolation of the arc operon of S. gordonii DL1. To prepare a subgenomic DNA library, total chromosomal DNA isolated from S. gordonii DL1 was digested to completion with various restriction enzymes. The digested DNA fragments were separated on a 0.8% agarose gel and analyzed by Southern blot analysis under stringent hybridization conditions using a 0.5-kbp PCR product internal to the S. gordonii arcB gene, which was generated as described in Materials and Methods. The results indicated that the arcB gene was comprised in an XbaI fragment of approximately 7-kbp. To isolate this fragment, a subgenomic DNA library of XbaI fragments was constructed on the moderate copy-number plasmid, pSU20 (Bartolome et al., 1991). The library was screened by colony hybridization under stringent condition with an arcB-specific probe. A positive clone, comprising a 7.0-kbp DNA insert (pJZ40), was identified. Southern blot analysis confirmed that the origin of this 7.0-kbp XbaI DNA fragment was S. gordonii and demonstrated that this fragment was contiguous on the chromosome.

The results of sequence analysis of the 7.0-kbp XbaI fragment indicated that this fragment comprised the 3′ portion of a partial open reading frame (ORF) that shared homology with other known arcA genes, followed by 5 complete ORFs. To obtain complete sequence of this partial ORF and potentially identify other genes tightly linked to the arginine catabolism cluster, a subgenomic DNA library of HincII fragments was constructed as described in Materials and Methods, and the library was screened with a 1-kbp DNA fragment comprising the 3′ portion of arcA. A 3-kbp HincII fragment was subsequently identified and the origin of this fragment was confirmed by Southern blotting of S. gordonii chromosomal DNA under stringent hybridization conditions.

Nucleotide sequence analysis of the arc operon. The complete nucleotide sequences of both strands of the 7.0-kbp XbaI fragment and the 3.0-kbp HincII fragment were determined from a series of nested deletions generated by exonuclease III. Translation of the nucleotide sequences revealed 6 ORFs that were arranged contiguously as an operon on the chromosome. The first 5 ORFs were transcribed in the same direction and the sixth was transcribed in the opposite direction. All 6 ORFs were preceded by a putative Shine-Dalgarno sequence and began with an ATG codon. A putative rho-independent terminator was located between ORF5 and ORF6. The ORFs were found to share significant homologies, and to correspond closely in molecular mass with previously characterized arc genes. FIG. 3 is a schematic diagram of the six arc genes, which were designated arcA, B, C, D, T and R. The complete nucleotide sequence of the ADS of S. gordonii, including an anerobic regulator flp is disclosed herein as SEQ ID NO:2.

The similarities between the deduced amino acid sequences of arcABCDTR of S. gordonii and arc genes of other bacteria were evaluated. Briefly, S. gordonii AD, encoded by arcA, shared greater than 92% and 84% identity to Streptococcus pneumoniae and Streptococcus pyogenes AD, respectively. Lesser, but still significant similarities were also observed with the AD enzymes of Enterococcus faecalis (65%), L. sakei (51%) and B. licheniforms (52%).

S. gordonii arcB, encoding cOTC, shared greater than 94% and 89% identity with S. pneumoniae and S. pyogenes cOTC, respectively. Lower, but still substantial levels of homology were observed with cOTC from Gram-negative microorganisms. The conserved motifs for carbamylphosphate binding and catalysis, i.e., STRTR and HPTQ, were identified at protein residues 58 to 62 and 136 to 139, respectively. Other conserved amino acid residues between these two regions that are essential for binding and catalysis were also present in the S. gordonii cOTC (K88, K89, R101, G110). The ornithine binding motif, i.e., LHCLP was located at amino acid residues 271 to 275.

S. gordonii CK shared 89% and 65% identity with S. pneumoniae and S. pyogenes CK, respectively. Similar to what was noted for the E. faecalis CK, the conserved motif for carbamyl phosphate binding in other CKs, i.e., STRTR, could not be identified in the S. gordonii CK, nor was the P-loop that is known to be involved in mononucleotide binding.

S. gordonii ArcD, the arginine-ornithine antiporter, shared 74% and 64% identity with ArcD of S. pneumoniae and S. pyogenes, respectively. Eleven transmembrane helices were predicted based on deduced amino acid sequence of S. gordonii arcD by using the dense alignment surface method (Cserzo et al., 1997). Finally, the ArcT was found to share 70% homology with an S. pyogenes Xaa-H is dipeptidase.

Lesser, but significant levels of homology, as compared to the enzymes of the ADS, were detected between ArcR of S. gordonii and S. pyogenes (65% identity). A substantial degree of similarity was also observed when S. gordonii ArcR was compared to other known arginine metabolism regulatory proteins, such as Bacillus subtilis AhrC (64%) and B. licheniformis ArgR (64%). Similar to B. subtilis AhrC and E. coli ArgR (Grandori et al., 1995), the pI for the first 100 aa of S. gordonii ArcR was basic (9.71) while the pI for the last 56 residues was acidic (3.75). Furthermore, the serine-arginine motif that has been shown to be essential for DNA-binding was found at the N-terminus of ArcR (amino acids 43-44). Conserved amino acid residues for arginine binding were found in S. gordonii ArcR at amino acid positions 103 (alanine), 125 (aspartic acid) and 126 (aspartic acid), and the conserved glycine residue for oligomerization was also present at amino acid 124.

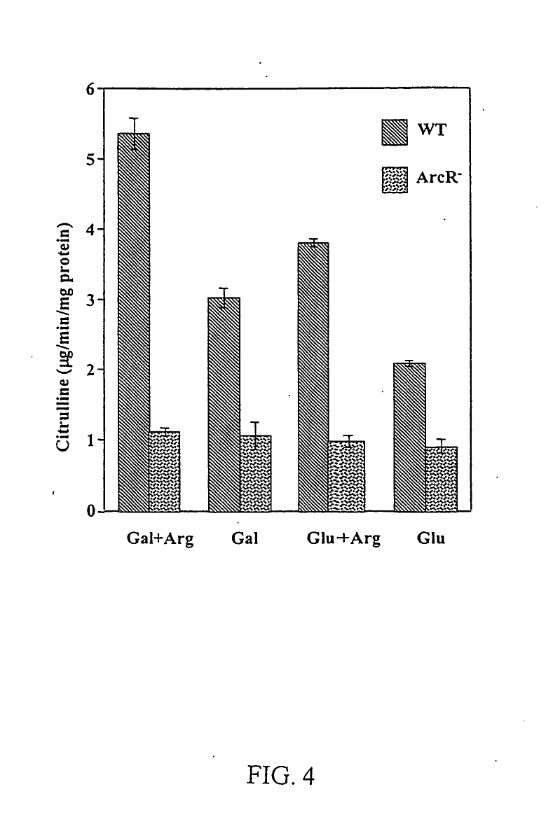

Expression of AD in S. gordonii. The ADS in lactic acid bacteria is highly regulated by growth conditions (Curran et al., 1998; Zuniga et al., 1998). Specifically, in S. sanguis NCTC 10904, the system requires induction by arginine and expression is subject to carbohydrate catabolite repression (CCR), with higher levels of activity in cells grown on non-repressing sugars, such as galactose. To investigate whether the expression of the S. gordonii DL1 arc operon was regulated in the same manner, AD specific activities in the wild-type strain were measured in cells grown in various energy sources (FIG. 4). When cells were grown in galactose, higher levels of AD activity were observed, as compared to cells grown in glucose. Addition of 1% arginine to the growth media resulted in a further increase in AD activity, although the presence of glucose had a repressive effect on AD expression. Thus, S. gordonii arcA was inducible by arginine and sensitive to CCR.

Localization of ParcA. To investigate the expression of the arc operon at the molecular level, the transcriptional initiation site of the most 5′ gene in the arc cluster, i.e., arcA, was first localized by primer extension with total cellular RNA isolated from cells grown under inducing and derepressing conditions (1% arginine and 10 mM galactose). A single product, 50 bases 5′ to the ATG start codon at a G residue, was observed, and a putative σ⁷⁰ promoter ParcA: TTGTGT-N₁₉-TAGAAT (SEQ ID NO:22) was identified at an appropriate distance 5′ to the signal.

CCR in low-GC gram-positive bacteria has been studied extensively (Stulke, 1999). The two major regulatory elements involved are the trans-acting factor, catabolite control protein A (CcpA), and a cis-acting palindromic sequence designated as a catabolite-responsive element (cre). In the presence of glucose, CcpA binds to CRE and represses the expression of catabolic genes and operons. Two cre sites were identified 5′ to the transcription initiation site. One site: AGAAAACGCTTCAA (SEQ ID NO:23), spanning from −107 to −94, differed by 3 bases from the consensus sequence. The other site: TGTAAGTGTTTTCA (SEQ ID NO:24) spanning from −35 to −22, differed by only one base compared to the consensus sequence, suggesting that the catabolite repression of ParcA may be controlled by CcpA. On the other hand, the Arg box, the cis-element essential for the activation of arginine-dependent anaerobic growth in B. licheniformis and P. aeruginosa (Maghnouj et al, 1998; Lu et al., 1999) was not found 5′ to arcA translation start site.

Functional analysis of ArcR. To determine whether ArcR was involved in the regulation of arc operon expression, an ArcR-deficient derivative was constructed by allelic exchange with the insertion of a Km resistant marker. The levels of the arcA message in both the wild-type and ArcR-deficient strains were evaluated by primer extension analysis under different growth conditions. In agreement with the AD activities observed in wild-type cells under different growth conditions, the expression was repressible by glucose and inducible by arginine, with the highest level of transcription of ParcA detected in wild-type cells grown in the presence of galactose and arginine. In the absence of ArcR, lower levels of expression were consistently observed when compared with wild-type cells growing on the same substrates, indicating that ArcR was required for optimal arc expression. Thus arcR was shown to be a positive regulator of arc gene expression. Similar to wild-type cells, expression from ParcA in the ArcR-deficient strain was still sensitive to CCR, with consistently lower levels of expression being observed when galactose was the sole carbohydrate source.

### Example 4

### Isolation of the Gene Cluster for the Arginine Deiminase System from Streptococcus rattus FA-1, a Noncariogenic Strain Closely Related to S. mutans

This example describes the isolation of another exemplary bacterial arginine deiminase system useful in the practice of the invention.

Materials and Methods:

Bacterial strains, growth conditions and reagents. S. rattus FA-1 was grown in brain heart infusion (BHI, Difco Laboratories, Detroit, Mich.) broth at 37° C., in 5% CO₂ and 95% air. Recombinant Escherichia coli strains were maintained on L agar supplemented, when indicated, with 25 μg ml⁻¹ chloramphenicol (Cm) or 50 μg ml⁻¹ kanamycin (Km). All chemical reagents were obtained from Sigma (St. Louis, Mo.).

DNA manipulations. Genomic DNA from S. rattus FA-1 was isolated as described above. Plasmid DNA was isolated from E. coli by the method of Birnboim and Doly (1976). Plasmid DNA used in sequencing reactions was prepared from E. coli DH10B using the QIAprep Spin Plasmid Kit (Qiagen Inc., Valencia, Calif.). Cloning and electrophoretic analysis of DNA fragments were carried out according to established protocols, and Southern hybridization and high-stringency washes were performed as described above. Restriction and DNA modifying enzymes were purchased from Life Technologies Inc. (Rockville, Md.) or New England Biolab (Beverley, Mass.).

To prepare subgenomic DNA libraries of S. rattus FA-1, total chromosomal DNA was digested to completion using XbaI or EcoRV. The digested DNA fragments were separated on agarose gels and the XbaI fragments of 6- to 8-kbp or EcoRV fragments of 4- to 5-kbp were enriched by gel purification using the Elu-Quik DNA Purification Kit (Schleicher & Schuell, Keene, N.H.). The isolated DNA fragments were ligated onto XbaI- or HincII-digested, phosphatase-treated pSU20 (Bartolome et al., 1991), respectively. The ligation mixtures were used to transform E. coli DH10B and Cm resistant transformants were selected.

Development of an arcB-specific probe. An internal fragment of the S. rattus FA-1 arcB gene, encoding cOTC, was generated by PCR using degenerate primers based on alignments of known anabolic and catabolic OTCs.

Primer arcBS: 5′-CAAGTATTTCAGGGACGC-3′ (SEQ ID NO:25) encoded amino acid residues 3 through 8 of cOTC.

Primer arcBAS: 5′-CATCTGTCAAGCCATTCC-3′ (SEQ ID NO:26) encoded the antisense sequence, corresponding to amino acid residues 127-132 of cOTC. Each reaction consisted of 25 cycles at a stringent annealing temperature (55° C.). The product with the correct predicted size was gel purified prior to cloning into pCR™II to generate pJZ22. Southern blot analysis confirmed hybridization of the PCR product to S. rattus FA-1 chromosome at high stringency. Sequence analysis and BLAST searches were performed to confirm that the product shared high degrees of homology with other cOTCs.

RNA isolation, primer extension and reverse-transcriptase PCR analysis. The protocol used to isolate total RNA from S. rattus FA-1 was the same as that originally described for Streptococcus salivarius RNA isolation (Chen and Burne, 1996). S. rattus FA-1 was grown to mid-exponential phase in TV medium comprising 2% glucose and 1% arginine. Primer extension analysis was used to map the arcA transcription initiation site. Primer ArcAS: 5′-GACGATGTAACATTACCTTCTT-3′ (SEQ ID NO:27) encoded the antisense sequence of arcA located 50 bases downstream from the translational start site. Incubation of radiolabeled primers with 50 μg of total RNA at 42° C. for 90 minutes was followed by reverse transcription, and the products were separated by electrophoresis and disclosed by autoradiography. A DNA sequencing reaction using the same primer was included on the gel to allow identification of the start site.

To determine if the arc genes of S. rattus FA-1 could be co-transcribed, reverse-transcriptase (RT) PCR was performed using the SuperScript First-Strand Synthesis System (Invitrogen, Carlsbad, Calif.). RNA was isolated from S. rattus FA-1 grown in TV+2% galactose and 1% arginine. PCR amplification of the cDNA was performed using various primer pairs:

(SEQ ID NO:28)

| | |
|---|---|
| arcABS: | 5′-CTGTGTATGTCTATGCCATTTG-3′ |

(SEQ ID NO:29)

| | |
|---|---|
| arcABAS: | 5′-AGCTAGGAAACTGCGTCCCT-3′ |

(SEQ ID NO:30)

| | |
|---|---|
| arcDTS: | 5′-TTTAGACTCTTTACAGGACAGATT-3′ |

(SEQ ID NO:31)

| | |
|---|---|
| arcDTAS: | 5′-TGAATATTCATCTGTTTACCCCTT-3′ |

(SEQ ID NO:32)

| | |
|---|---|
| arcTRS: | 5′-AGTGAGTTGTCTGAGTTTCTA-3′ |

(SEQ ID NO:33)

| | |
|---|---|
| arcTRAS: | 5′-TTTATCTTACTTTGGCGCAATA-3′. |

These primer pairs flanked the intergenic region of arcAB, arcDT and arcTR, respectively.

Construction of promoter fusions and CAT assays. The arcA promoter and deletion derivatives were amplified via recombinant PCR (Higuchi, 1990) using sense primers:

arcASacI-S400: TTGCTCTAGAGCTCTCAAATGACAGAA (SEQ ID NO:34)

arcASacI-S150: TTATAAATTCGAGCTCCAAAAAACGTGAA (SEQ ID NO:35);

arcASacI-S100: TAAATAACAATTCGAGCTCGAAAAAAATCTTA (SEQ ID NO:36) in conjunction with the antisense primer

arcABamHI-AS: TTTTGAGTCATGGATCCTACTCCTTTCGAT (SEQ ID NO:37). These primers allowed insertion of SacI and BamHI restriction sites (indicated in bold) to facilitate cloning.

The PCR products were ligated to the 5′ end of a promoterless chloramphenicol acetyltransferase gene (cat) from Staphylococcus aureus, and cloned onto the integration vector pMJB8A (Chen et al., 2002). The constructs were then electroporated into E. coli DH10B, screened for the correct configurations and then introduced into S. gordonii DL-1 by natural transformation. S. rattus FA-1 cultures were grown in TV medium comprising 2% glucose or galactose, with or without 1% arginine, to an optical density at 600 nm=0.6.

Biochemical assays. S. rattus FA-1 was grown in the TV medium supplemented with 0.2-2% glucose or galactose±1% arginine at 37° C. in 5% CO₂ and 95% air. AD activity was measured by monitoring production of citrulline from arginine, as described above. Chloramphenicol acetyltransferase assays were performed using the spectrophotometric method of Shaw (1979). Enzyme activities were normalized to protein concentration, which was determined by the method of Bradford using a kit (BioRad) with BSA as the standard.

Results:

Isolation of the arc genes of S. rattus FA-1. To prepare a subgenomic DNA library, total chromosomal DNA isolated from S. rattus FA-1 was digested to completion using various restriction enzymes. The digested DNA fragments were separated on a 0.8% agarose gel and screened for the presence of arcB by stringent hybridization with a 0.35-kbp PCR product internal to the S. rattus arcB gene. Results indicated that the arcB gene was comprised on an XbaI fragment of approximately 7-kbp. To clone this fragment, a subgenomic DNA library of XbaI fragments was constructed in the intermediate copy-number plasmid, pSU20. The library was screened by colony hybridization under stringent conditions with an arcB-specific probe.

A positive clone, comprising a 7.0-kbp DNA insert (pJZ29), was identified. Southern blot analysis confirmed that the 7.0-kbp XbaI DNA fragment originated from S. rattus and demonstrated that the fragment was continuous on the chromosome.

Results of DNA sequence analysis performed on the 7.0-kbp XbaI fragment indicated this fragment comprised the 3′ portion of a partial open reading frame (ORF) that shared homology with other known arcA genes and was followed by five complete ORFs. To obtain genomic DNA fragments comprising the complete ORF, and potentially identify other genes tightly linked to the arginine catabolism cluster, a subgenomic DNA library of blunt-ended EcoRV fragments was constructed and screened with a 1-kbp DNA fragment comprising the 3′ portion of arcA. A 4-kbp EcoRV fragment was subsequently isolated, and a Southern blot analysis under stringent conditions confirmed that the fragment originated from S. rattus.

Nucleotide sequence analysis of the arc genes. Using a series of nested deletions generated by exonuclease III, the complete sense and antisense nucleotide sequences of the 7-kpb XbaI fragment and 4-kpb EcoRV fragment were determined. The nucleotide sequences were translated and found to encode six ORFs arranged in an apparent operon. Each ORF began with an ATG start codon and was preceded by a putative Shine-Dalgarno sequence. Between the fifth and sixth ORF, a stable stem:loop structure possibly acting as a terminator was identified. Based on similarity to known arc genes, the ORFs were designated arcA, B, C, D, T and R. The complete nucleotide sequence of the ADS of S. rattus is shown herein as SEQ ID NO:3.

The predicted amino acid sequences of the six ORFs were determined and BLAST searches were used to identify sequence similarity to other known proteins. S. rattus AD, encoded by arcA, was 85, 84, 80 and 79% identical to the AD enzymes of Streptococcus agalactiae, Streptococcus pyogenes, Streptococcus pneumoniae and S. gordonii, respectively. S. rattus AD also shared homology with the AD proteins of Enterococcus faecalis, Clostridium perfringens, Bacillus licheniformis, Lactobacillus sakei and Staphylococcus aureus. Several conserved regions were identified in S. rattus ArcA, including the signature arginine deiminase motifs at amino acids 11-21, 164-167, 220-223, and 274-280, as previously described (Knodler et al., 1998).

S. rattus cOTC, encoded by arcB, shared 88, 86, 85 and 85% identity with the OTCases of S. pyogenes, S. agalactiae, S. gordonii and S. pneumoniae, respectively. Additional homologies were observed with cOTC enzymes from E. faecalis, L. sakei, B. licheniformis and S. aureus. Conserved carbamoylphosphate binding and catalysis motifs were identified at amino acid residues 57-61 and 135-138, respectively as described (Houghton et al., 1984). A conserved ornithine binding site (LHCLP) was identified at positions 270-274.

Carbamate kinase, encoded by S. rattus arcC, was 73 and 72% identical to that of S. gordonii and S. pneumoniae, respectively. Homologies were also observed with the carbamate kinases of Listeria monocytogenes, S. pyogenes, S. agalactiae, S. mutans, L. sakei, and B. licheniformis. Aside from the highly conserved arginine residues at amino acid residues 157 and 160, no other conserved motifs were identified in the S. rattus arcC, as reported for S. gordonii and E. faecalis.

The arginine/ornithine antiporter encoded by S. rattus arcD shared 49 and 42% identity with those of L. sakei and S. aureus, respectively. Conservation was also observed with the arginine/ornithine antiporters of S. agalactiae, C. perfringens and Pseudomonas putida. Twelve predicted transmembrane helices were identified in ArcD of S. rattus using the dense alignment surface method from DAS-Transmembrane Prediction Server (Czerzo et al., 1997).

S. rattus arcT, encoding a putative peptidase, shared 58% identity with the ArcT of L. lactis and 59% identity with SMU.816, a putative aminotransferase of S. mutans UA159. Additional homologies were observed with the ArcT proteins of Lactobacillus lactis and L. sakei, as well as a Lactobacillus plantarum aminotransferase.

The arc operons of other bacteria, including P. aeruginosa, S. gordonii, B. licheniformis and E. faecalis, are activated during anaerobic conditions by ArcR, a Crp/Fnr-type transcriptional regulator. In these bacteria, ArcR activates the arc genes by binding to a Crp-like consensus sequence upstream of the arcA promoter. The predicted amino acid sequence of S. rattus ArcR was analyzed to determine if any such conserved DNA or arginine binding residues were present. The conserved SR--RE motif thought to be involved in DNA contact was found at amino acid residues 44-49 of the S. rattus ArcR, consistent with involvement of the N-terminus of ArcR in DNA binding. Conserved amino acid residues thought to be important for important for arginine binding were identified near the C-terminus at positions 101 (alanine) and 124 (aspartic acid). In addition, a conserved glycine residue involved in oligomerization was identified at position 122.

The amino acid sequence of S. rattus ArcR was compared to those of arginine metabolic regulatory proteins found in other bacteria. The most significant level of similarity was observed with the putative S. mutans UA159 ArgR (80% identity). The second highest identity was with ArgR of S. agalactiae (58%), followed by putative arginine repressors in S. pyogenes and S. pneumoniae, as well as AhrC of L. lactis. The AhrC protein was originally identified in Bacillus subtilis as a repressor of the arginine biosynthetic genes. A homologue, ArgR, has been identified in Bacillus licheniformis, where it activates the arc operon in the presence of arginine by binding to a conserved ARG box located upstream of the arcA promoter.

The sequence upstream of S. rattus arcA was analyzed for possible ArgR or ArcR DNA binding sites. Potential binding sites for both ArgR and ArcR (Grandori et al., 1995; Tian and Maas, 1994) were identified 200 and 52 bases upstream of the arcA transcriptional start site, respectively. The presence of a putative Arg box and an imperfect palindrome possibly constituting a Crp binding site matching E. coli consensus sequences (Maas, 1994) suggest that ADS in S. rattus may be under the control of an ArgR/AhrC-type protein in addition to a Crp/Fnr family member, most likely the S. rattus ArcR protein.

Localization of P_{arcA} and reporter gene fusions. Primer extension analysis was used to map the arcA promoter region. A single band was observed corresponding to a G residue 49 bases upstream of the arcA start codon. Examination of the upstream sequence revealed a putative promoter with some that was most similar to σ⁷⁰-type promoters. The −10 region shared 5 out of 6 bases with the consensus sequence, whereas the −35 region identified 17 bases upstream of the −10 region shared only 3 bases with the consensus, although reporter gene studies confirm the presence of a functional promoter in this region.

To determine which arc genes could be transcribed from the arcA promoter, RT-PCR analysis was performed on RNA isolated from S. rattus FA-1 grown in TV+2% galactose+1% arginine. RT-PCR results suggested the presence of a polycistronic arcABCDT transcript. No transcript could be detected between the intergenic region of arcT and arcR, suggesting that arcR is transcribed from a separate promoter.

Expression of the arc operon in Lactobacillus sakei (Zuniga et al., 1998), as well as some oral streptococci, is under the control of carbohydrate catabolite repression (CCR). ADS expression in these bacteria appears to be up-regulated in the presence of arginine and repressed by glucose. In AT-rich gram-positive bacteria, CCR is mediated by the trans-acting catabolite control protein A (CcpA), which binds to cis-acting catabolite response elements (cre) in the presence of preferred carbohydrate sources to regulate the expression of catabolic genes and operons (Saier et al., 1996). Two potential CcpA-dependent cre were identified at −58 and −107 relative to the S. rattus P_{arcA}. The presence of these elements is consistent with the observation that the ADS in S. rattus FA-1 is regulated by CCR (Burne et al., 1991).

To assess the functionality of P_{arcA} and the putative cre sites found upstream of the promoter region, p_{arcA} and deletion derivatives were fused to the intact cat gene from S. aureus. Three arcA promoter fusions were constructed: (1) 400 bases upstream of the ATG start codon, including both putative cre sites, (2) 150 bases upstream, including one cre site, and (3) 100 bases upstream, lacking both cre sites. All of the promoter fusions were constructed such that expression of cat was driven by the cognate arcA RBS. Since an efficient system of genetic transformation has not yet been established for S. rattus FA-1, the promoter fusions were integrated into the gtfG gene on the chromosome of the naturally competent, ADS-positive organism, S. gordonii DL-1 using a previously described integration vector (Chen et al., 2002). Results showed that the intact p_{arcA} was functional in S. gordonii, and deletion of the putative cre sites resulted in increased promoter activity relative to the wild-type, providing evidence that expression from p_{arcA} is regulated via CcpA-dependent catabolite repression.

Expression of AD in S. rattus. The identification of putative cre sites upstream of p_{arcA}, as well as the ADS regulation patterns observed in S. gordonii, prompted an investigation of the role of catabolite repression in S. rattus FA-1 arc regulation. Wild-type S. rattus FA-1 was grown in TV broth comprising glucose or galactose with or without supplemental arginine to mid-log phase, and AD activity was measured. Galactose was included in the analysis because it is not repressive for AD expression in S. gordonii.

In wild-type S. rattus grown in 2% carbohydrate, peak AD activity was found in cells grown in galactose and arginine, while expression decreased by 75% in cells grown in glucose, regardless of the presence of arginine. Catabolite repression of ADS in S. rattus FA-1 was not evident when the amount of glucose or galactose present in the growth medium was decreased to 0.2%. This differs from CCR of the ADS in other closely related oral streptococci, including S. gordonii, where inclusion of as little as 0.2% of a preferred carbohydrate source in the growth medium strongly represses ADS expression.

### Example 5

### Production of Recombinant Bacteria Expressing Arginine Deiminase Gene Cluster

The general strategy for construction of arginine deiminase positive plaque streptococci is essentially similar to that detailed for the urease recombinant strains, as is the design for each vector. A commercially available E. coli plasmid cloning vector comprising the 5′ and 3′ fragments flanking the desired insertion site can be used. For laboratory applications, those sequences can flank an antibiotic resistance marker (generally kanamycin, tetracycline or erythromycin) and a unique cloning site(s) into which the foreign genes are cloned. Standard cloning protocols are used to introduce the arginine deiminase genes, for example arcABCDTR, flp from S. gordonii DL1 (SEQ ID NO:2), driven by the cognate arc and flp promoters, or by a stronger heterologous promoter, into a site between the 5′ and 3′ fragments of the target site. Similarly, the arginine deiminase genes arcABCDTR isolated from S. rattus FA-1 (SEQ ID NO:3) can be used.

Confirmation that the genes are correctly inserted is performed by standard methods. Then, the plasmid can be digested with restriction enzyme(s) and the DNA then used to transform plaque streptococci that have been prepared to accept DNA by standard methods. Selection for antibiotic resistance, if appropriate, and subsequent confirmation that the genes are integrated and that the strains express the desired activities are performed. For strains that may be suitable for human applications, the antibiotic resistance is omitted and strains are screened for their capacity to produce ammonia or other alkalis from the appropriate substrates, as described above.

### Example 6

### Isolation of Agmatine Deiminase Gene Cluster from S. mutans UA159

This example describes the isolation of an exemplary bacterial agmatine deiminase system useful in the practice of the invention.

Materials and Methods:

Bacterial strains, growth conditions and reagents. S. mutans UA159 was grown in brain heart infusion broth (BHI, Difco Laboratories, Detroit, Mich.) at 37° C., in 5% CO₂ and 95% air. To monitor AgDS expression, wild-type S. mutans UA159 was grown in TV medium (Burne et al., 1999) comprising 0.5% glucose or galactose, with or without 10 mM agmatine. Recombinant E. coli strains were maintained on L agar supplemented, when indicated, with 1 mg ml⁻¹ kanamycin (Km). Chemical reagents were obtained from Sigma (St. Louis, Mo.).

RNA analyses. RNA was prepared from cells harvested at OD₆₀₀≅0.6 and immediately treated with the RNAprotect® reagent from Qiagen (Qiagen Inc., Valencia, Calif.). Total RNA was isolated using protocols described elsewhere (Chen and Burne, 1996). The RNA was further purified and treated with DNaseI using the RNeasy RNA Clean Up mini kit from Qiagen, and stored at −80° C.

To determine if the gene cluster was expressed, the SuperScript First-Strand Synthesis System (Invitrogen, Carlsbad, Calif.) was used to conduct reverse transcriptase-PCR (RT-PCR) analysis. First-strand cDNA synthesis was completed using random hexamers. PCR amplification of the first strand aguB cDNA was performed using primer pairs:

(SEQ ID NO:38)

| | |
|---|---|
| aguBS: | 5′-CAGATTATATCTAGACAGAGGATTT-3′ |
| and | |

(SEQ ID NO:39)

| | |
|---|---|
| aguBAS: | 5′-TACCAGCTGGGAATCCTTCTATCATTGTA-3′. |

Comparisons of the amounts of AgDS mRNA in cells grown under different growth conditions were performed by slot blot analysis. Samples comprising 1, 2.5 or 5 μg total RNA, as well as a 5 μg sample of RNaseA-treated RNA as a negative control, were UV-crosslinked to a 0.45 micron nitrocellulose membrane. The DNA probe was labeled using the Ambion Bright Star Psoralen-Biotin nonisotopic labeling kit (Ambion Inc, Austin, Tex.). Hybridizations were carried out according to the supplier's recommendation under high stringency conditions.

DNA manipulations and construction of a polar aguB mutant. Genomic DNA was isolated from S. mutans UA159 as previously described (Chen and Burne, 1996). Plasmid DNA used in sequencing reactions was prepared from E. coli DH10B using the method of Birnboim and Doly (1979). Cloning and electrophoretic analysis of DNA fragments were carried out according to established protocols. Southern hybridization and high-stringency washes were performed as previously described (LeBlanc and Hassell, 1976). Restriction and DNA modifying enzymes were purchased from Life Technologies Inc. (Rockville, Md.) or New England Biolabs (Beverly, Mass.).

Recombinant PCR was used to construct a polar aguB mutant. The first half of aguB was amplified using primer pairs:

aguBSXbaI: 5′-CAGATTATATCTAGACAGAGGATTT-3′ (SEQ ID NO:40) and

aguBASEcoRI: 5′-TACCAGCTGGGAATTCTTCTATCATTGTA-3′ (SEQ ID NO:41), which inserted XbaI and EcoRI restriction sites (indicated in boldface).

The remaining portion of aguB was amplified using primer pairs:

aguBSEcoRI: 5′-TACAATGATAGAAGAATTCCCAGCTGGTA-3′ (SEQ ID NO:42) and

aguBASSstI: 5′-ACCGTCCATGAGCTCATCTGTAATCT-3′ (SEQ ID NO:43), which inserted EcoRI and SstI restriction sites (boldface) to facilitate cloning.

The PCR fragment was then cloned onto pGEM7z(+) and electroporated into E. coli DH10B. The construct was then digested with EcoRI and an Ω kanamycin cassette harboring strong transcription/translation termination signals (Perez-Casal et al., 1991) was inserted at the EcoRI restriction site to disrupt aguB. The construct was transferred into S. mutans, selecting for growth on BHI agar comprising 1.0 mg ml⁻¹ Km. Correct integration was confirmed by Southern blot analysis.

To develop an aguB-specific probe, an internal fragment of S. mutans UA159 aguB, encoding a putative putrescine carbamoyltransferase, was PCR amplified using primer pair:

aguBS: 5′-CAGATTATATCTAGACAGAGGATTT-3′ (SEQ ID NO:44) and

aguBAS: 5′-TACCAGCTGGGAATCCTTCTATCATTGTA-3′ (SEQ ID NO:45). Each PCR reaction consisted of 25 cycles at a stringent annealing temperature (55° C.). Southern blot analysis confirmed hybridization of the PCR product to the S. mutans UA159 chromosome at high stringency.

Agmatine deiminase assays. AgD activity was measured by colorimetric determination of N-carbamoylputrescine production from agmatine as described (Archibald, 1944). S. mutans strains were grown in BHI supplemented with 10 mM agmatine or TV medium comprising 2% glucose or galactose, with or without 10 mM agmatine, to an optical density at 600 nm of 0.6. Cells were harvested by centrifugation, washed once with 10 mM Tris-maleate buffer, pH 6.0, and resuspended in 1/10 of the original culture volume in the same buffer. The cells were permeabilized using 1/20 volume of toluene and two, one-minute freeze-thaw cycles. The cell suspension was centrifuged and the pellet was resuspended in 500 μl of 10 mM Tris-maleate, pH 6.0. A 50 μl aliquot of the cell suspension was used in a 500 μl reaction mixture comprising 10 mM agmatine or 50 mM arginine, when specified. After 30 minutes, reactions were terminated by the addition of an equal volume of 10% trichloroacetic acid and N-carbamoylputrescine was measured.

The protein concentration of the cell suspension was determined as follows. A known volume of the cell suspension was mixed with an equal volume of glass beads (0.1 mm) and homogenized using a Bead Beater. The samples were centrifuged for 10 min in a refrigerated microcentrifuge and the protein concentration of the lysate was measured using a protein assay (Bio-Rad, Hercules, Calif.) based on the method of Bradford (1976) with bovine albumin serum as the standard. AgD activity was expressed as nmol N-carbamoylputrescine min⁻¹ (mg protein)⁻¹.

Ammonia production from agmatine was measured in intact cells of S. mutans UA159 grown to mid-exponential phase (OD₆₀₀≅0.6). The cells were collected by centrifugation, washed with potassium phosphate buffer, pH 7.0, and resuspended in 1/10 the original culture volume of the same buffer. A 10 μlaliquot of the cell suspension was used in a reaction mixture comprising 50 mM potassium phosphate, pH 7.5, and 10 mM agmatine. The reaction was carried out at 37° C. for 30 minutes and ammonia production was measured using an Ammonia Detection kit (Diagnostic Chemicals Limited, Charlottetown, Canada), which allows determination of ammonia concentration by monitoring the rate of NADP production in a glutamate dehydrogenase-catalyzed reaction: NH₄⁺+α-ketogluterate+NADH→L-glutamate+NADP⁺+H₂0. The protein concentration of cell suspensions was determined as described above. Ammonia production was expressed as nmol NH₄⁺ min⁻¹ (mg protein)⁻¹. Ammonia production by intact cells of S. mutans at various pH values were carried out as described above, except different buffers were used (glycine-HCl<pH 4.5; Tris-maleate>pH 4.5; potassium phosphate buffer >pH 7.0). Reactions were carried out for 60 minutes. Appropriate controls were conducted to ensure that inclusion of different buffers did not alter the ability to measure ammonia using the coupled assay.

Results:

Analysis of the DNA sequence of the agmatine deiminase gene cluster. The S. mutans AgD enzyme, encoded by SMU.264, was found to share significant homology with both predicted and established agmatine deiminases in at least 10 other bacteria, including the well-characterized AgD of P. aeruginosa (Nakada et al., 2001). However, the remaining genes in the S. mutans ADS most closely resembled those of E. faecalis, L. sakei, L. monocytogenes and L. lactis with respect to operonic organization and predicted amino acid sequences. Of these bacteria, only E. faecalis has been fully established as possessing a functional agmatine deiminase system (Simon and Stalon, 1982).

The first gene in the operon, annotated as otcA, encodes a putative PTC and was redesignated aguB. AguB is 80% identical to ArgF-2, a putative OTC in E. faecalis V583. A lower level of similarity was evident between the S. mutans AguB and L. lactis subsp. lactis OtcA (78% identity), L. monocytogenes LMO036 (65%), and Lactobacillus sakei ArgF (62%). ArgF-2 is one of two putative OTCs in the E. faecalis genome. According to Prosite, a consensus pattern for carbamoyltransferases is F-x-[E/K]-x-S-[G/T]-R-T, with the third residue allowing differentiation between aspartate carbamoyltransferase (E) or OTC (K) enzymes. This consensus sequence is present in the predicted amino acid sequence of S. mutans aguB, as well as E. faecalis ArgF-2, L. lactis subsp. lactis OtcA, L. monocytogenes LMO0036, and L. sakei ArgF, but a highly conserved Q is present in the third position, perhaps reflecting the preference of the enzyme for putrescine, rather than aspartate or ornithine. The HPTQ residues at positions 143-146 known to be involved in carbamoylphosphate binding in OTCases are present in AguB, as are the HCLP residues at positions 281-284, which are conserved in OTCases and facilitate ornithine binding.

The second gene in the operon, annotated SMU.263, encodes a putative amino acid antiporter and was designated aguD. AguD is similar to proteins found in the putative AgDS gene clusters of other bacteria. AguD is 68% identical to E. faecalis EF0733, a predicted amino acid permease, 58% identical to the amino acid antiporter YrfD in L. lactis subsp. lactis, 46% identical to the amino acid transporter LMO0037 in L. monocytogenes, and 25% identical to the probable amino acid permease PA4804 in P. aeruginosa. Similar to other amino acid antiporters, 11 transmembrane helices were predicted for the amino acid sequence of AguD using the “DAS”-Transmembrane Prediction server (Czerzo et al., 1997).

The third gene, annotated as SMU.264, encodes AgD and was designated as aguA. S. mutans AguA shares significant homology with the known and putative AgD enzymes of at least 10 other bacteria. As demonstrated by the CLUSTAL W program (Thompson et al., 1994) amino acid sequence alignments, several regions are highly conserved in AgD enzymes belonging to S. mutans, E. faecalis, L. lactis subsp. lactis, and L. monocytogenes, while other regions are conserved in all 10 bacteria. In particular, two conserved regions are obvious, the first occurring at residues 120-137 in S. mutans and the second at residues 157-172. The GGGNIHCITQQ sequence noted elsewhere (Nakada et al., 2001) was identified at the C-terminus of all 10 AgD enzymes. S. mutans AguA shares the highest level of identity with YrfC in L. lactis subsp. lactis (66%), followed by conserved hypothetical protein EF0734 in E. faecalis (640%), which is most likely the AgD identified and characterized by Simon and Stalon (1982). Lower levels of homology were observed with LabD in L. sakei (56%) and AguA in P. aeruginosa (54%). SMU.264 is 56% and 52% identical to conserved hypothetical proteins LMO0038 and LMO0040 in L. monocytogenes, respectively.

The final gene in the S. mutans AgD gene cluster, annotated arcC, codes for a carbamate kinase that shares homology with carbamate kinases of Streptococcus agalactiae (61%), Streptococcus suis (61%), Streptococcus pyogenes (60%), L. monocytogenes (56%), E. faecalis (54%), and L. sakei (50%). This gene should be redesignated as aguC to reflect involvement of the protein in the AgDS, rather than the ADS.

The S. mutans AgDS genes, designated as aguB, aguD, aguA and aguC, are believed to encode a putative putrescine carbamoyltransferase, amino acid antiporter, agmatine deiminase and carbamate kinase, respectively. The complete nucleotide sequence of the AgDS gene cluster of S. mutans, herein designated as aguBDAC, is shown as SEQ ID NO:4. In addition to these genes, there is an ORF encoding a transcriptional regulator of the LuxR family located 239 base pairs upstream of aguB and transcribed in the opposite direction. It is possible that this protein is involved in regulation of the AgDS in S. mutans. A putative nitroreductase and an ABC transporter are located upstream of this regulatory protein, however no sensor kinase is present. Potential homologs of this regulatory protein were also identified 399 and 234 base pairs upstream of the AgDS gene clusters in E. faecalis and L. lactis subsp. lactis, respectively. The ORF located immediately upstream of aguB may play a negative regulatory role in the regulation of AgDS in S. mutans, similar to that of aguR in P. aeruginosa.

AgDS expression in S. mutans. Using RT-PCR analysis of mRNA from cells grown in TV comprising 0.5% galactose and 10 mM agmatine, it was demonstrated that cDNA can be amplified from aguB, providing evidence that the AgDS gene cluster is transcribed in S. mutans UA159. To quantify mRNA under different growth conditions, slot blot analysis was used. Total RNA was extracted from cells grown in TV broth supplemented with 0.5% glucose or

0.5% galactose, with or without 10 mM agmatine. Known amounts of RNA were transferred to a nitrocellulose membrane and probed with an aguB probe. The results showed that AgDS-specific mRNAs were detected under all growth conditions, although expression was several-fold higher when cells were grown in medium comprising 10 mM agmatine.

AgD activity in different growth conditions was measured by calorimetric determination of N-carbamoylputrescine production from agmatine. In wild-type S. mutans, peak AgD activity was observed in cells grown in galactose and agmatine, while activity decreased around 65% in cells grown in glucose and agmatine. No activity was observed when cells were grown without agmatine. The pattern of expression of enzyme activity was consistent with the mRNA analysis, indicating that agmatine is necessary for induction of the system.

A polar aguB mutation was constructed as described above, and assayed for production of N-carbamoylputrescine from agmatine. This mutation abolished production of N-carbamoylputrescine, further suggesting that the genes are involved in agmatine utilization and are probably transcribed from a single promoter. To determine if AgD was specific for agmatine, wild-type S. mutans was grown under inducing conditions and assayed for the ability to produce citrulline from arginine. Citrulline was not detected even after the reaction time was extended to several hours, suggesting that AgD is specific for agmatine.

Consistent with AgD enzyme activity, ammonia production from agmatine was only observed in cells grown in media comprising agmatine. In addition, ammonia production increased about two-thirds in wild-type S. mutans grown in galactose and agmatine, as compared to those grown in glucose and agmatine. Ammonia production by the AgDS was approximately two-fold higher than N-carbamoylputrescine production, consistent with the fact that one mole of agmatine yields two moles of ammonia. Polar mutation of the gene cluster eliminated ammonia production from agmatine.

The optimum pH for ammonia production appears to be around pH 6.0, although the system is capable of producing ammonia at pH values as low as 4. Production of ammonia within this range implies that the system has the potential to confer a competitive advantage over other oral bacteria during periods when S. mutans lowers the pH of dental plaque.

### Example 7

### Urease-Producing Streptococci Promote Stability of a 10-Species Oral Biofilm

Materials and Methods:

Oral biofilm model. A modification of a previously described 10-species consortium (Bradshaw et al., 1996; Kinniment et al., 1996) was used in this study. In this system, stable biofilm communities form, with all 10 species being well represented unless a significant stress is placed on the populations, for example by lowering of the pH or subjecting the populations to repeated carbohydrate pulses without pH control. As a result of such stresses, community diversity is lost and instead the biofilms are dominated by aciduric lactobacilli and streptococci, and lactate-metabolizing Veillonella dispar.

The biofilm system was modified in this study by the replacement of two non-ureolytic strains of the original 10-species consortium with two urease-producing strains. Specifically, Streptococcus sanguis was replaced with urease-producing Streptococcus salivarius 57.I (Chen et al., 1996), and Actinomyces viscosus was replaced with the urease-producing Actinomyces naeslundii WVU45 (Morou-Bermudez and Burne, 1999). Two otherwise isogenic, urease-negative derivatives of the input Streptococcus salivarius (Chen et al., 2000) and Actinomyces naeslundii (Morou-Bermudez and Burne, 1999) strains were used as controls.

Additionally, Streptococcus mutans UA159, a strongly cariogenic organism that can be genetically manipulated, replaced the S. mutans strain in the previously described consortium. Thus, the modified consortium comprised eight strains of non-ureolytic bacteria, i.e., Streptococcus mutans UA159, Streptococcus oralis SK193, Lactobacillus rhamnosus ATCC7469, Neisseria subflava A1078, Veillonella dispar ATCC17745, Prevotella nigrescens T588, Fusobacterium nucleatum ATCC10953, and Porphyromonas gingivalis W50, as well as two urease-producing strains, i.e., Streptococcus salivarius 57.I and Actinomyces naeslundii WVU 45.

Cultivation of bacterial consortium. The 10-species consortium was cultivated in a constant-depth film fermentor (CDFF) known to be useful for study of oral biofilm ecology (Kinniment et al., 1996; Wimpenny, 1997). Each strain was grown individually in liquid media to late exponential phase and 5 ml of each culture was combined, gently mixed and inoculated into the CDFF through a pump over a 6 hour period. Concentrations of input organisms were carefully controlled for each run, with bacterial counts ranging from 6.0×10⁷ to 8.3×10⁸ CFU ml⁻¹.

The biofilms were cultivated during the inoculation period and thereafter in a mucin-comprising medium, i.e., BMM (Bradshaw et al., 1989), comprising 2.5 g⁻¹ porcine gastric mucin, 2 g l⁻¹ proteose peptone, 2.5 g l⁻¹ KCl, 1 g l⁻¹ yeast extract, 1 g l⁻¹ trypticase peptone, 0.1 g l⁻¹ cysteine hydrochloride, and 0.001 g l⁻¹ haemin, or in BMM supplemented with 10 mM glucose (BMMG) or with 10 mM glucose and 10 mM urea (BMMUG). In all cases, the pH of the media was adjusted to pH 7.5 prior to autoclaving. Subsequent to the inoculation period, BMMUG was pumped into the CDFF at a rate of 100 ml h⁻¹ for seven days. Then, depending on the particular test, addition of BMMUG or BMMG was continued until day 11 at the same flow rate. The CDFF was housed in a 37° C. warm room under aerobic conditions.

Biofilms were grown on 4.75 mm diameter polytetrafluoroethylene (PTFE) plugs located in 15 PTFE sample pans, which were inserted into a rotating stainless-steel turntable as described (Kinniment et al., 1996). Each pan comprised 5 plugs that were recessed to a depth of 300 μm and the thickness of the biofilms was maintained by a scraper bar with the turntable rotating at 3 RPM. Samples were taken for culture analysis using the inocula, and from the biofilms formed on PTFE plugs at 7, 9, 10, and 11 days post inoculation. To sample the biofilms, a sample pan was aseptically removed from the fermentor and, using sterile forceps, plugs were carefully extruded with the biofilm still intact on the surface. The biofilms on the plugs were dispersed in 5 ml of 0.5% proteose peptone, 0.25% KCl by vortexing for 10 seconds and sonicating for 10 seconds at low power (Sonic Dismembrator, Model 100, Fisher Scientific). The cell suspensions were serially diluted in the same buffer and plated on a range of selective and non-selective media as previously described (Bradshaw et al., 1996; Kinniment et al., 1996). After sampling each of the 5 plugs, the PTFE sample pan was placed into 10 ml of sterilized deionized water, vortexed, and the pH of the suspension was recorded.

Results.

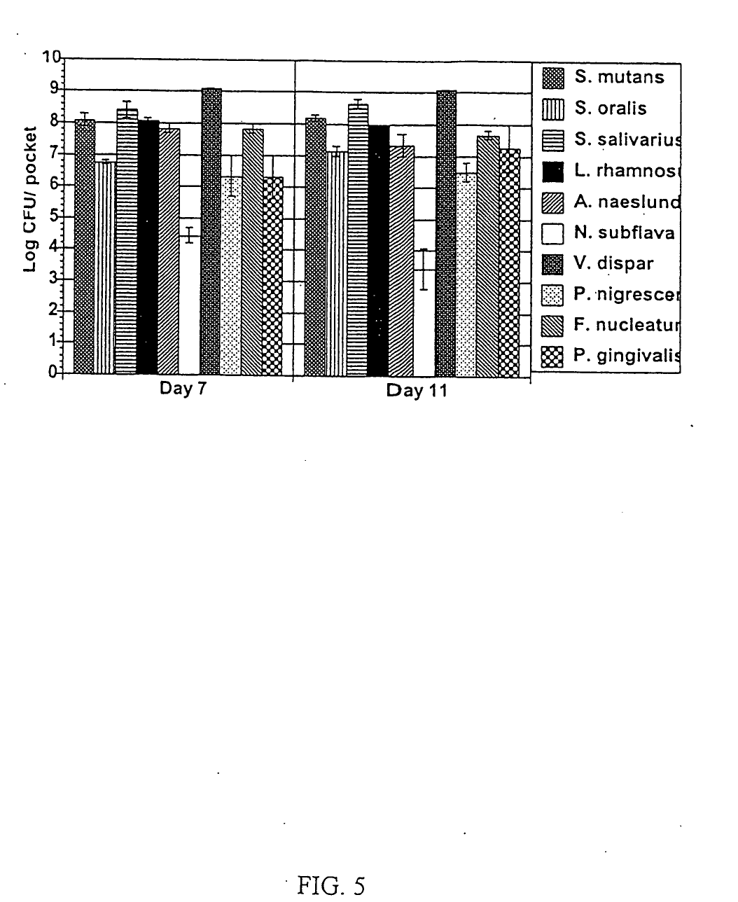

The role of urea. To determine whether urea could be used as a primary substrate to induce stability of the 10-species model, the CDFF was inoculated as described above with all 10 species, and biofilms were allowed to form for 7 days. At that time, sample plugs were removed, biofilm constituents were quantified by plating as previously described, and the pH of the biofilms was determined. Referring to FIG. 5, when the biofilms were allowed to form in BMMUG medium, all 10 species were well represented in the consortium. The bulk of the biofilms were constituted by the streptococci, and by Actinomyces, Lactobacillus and Veillonella, presumably due to the relatively high concentration of glucose added to the base medium. By contrast, the anaerobes, i.e., P. gingivalis, F. nucleatum and P. nigrescens, and the obligate aerobe N. subflava, were present, but at about 2- to 4-logs lower levels than the most abundant organisms, respectively. Further sampling at day 11 demonstrated that all ten species thrived, and that the composition of the community remained stable (FIG. 5). The pH of the biofilms at day 7 was 6.06 and was 5.91 at day 11.

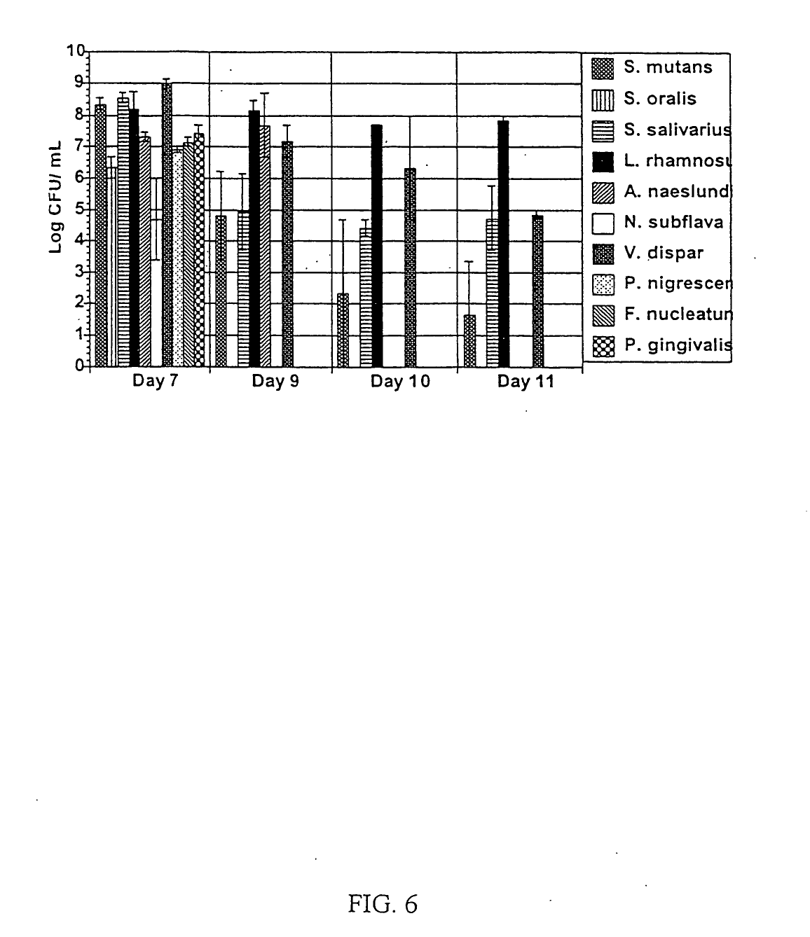

These results were in marked contrast to those obtained when the biofilms were formed in the presence of urea (BMMUG) for seven days, then switched to urea-free growth medium (BMMG). As expected, the biofilm composition after seven days was identical to that seen in the initial experiments (FIG. 6). However, after two days of incubation with BMMG as the nutrient source, the community diversity was dramatically affected. P. gingivalis, P. nigrescens, F. nucleatum, N. subflava and S. oralis were no longer cultivable from the biofilms. By day 10, all that remained were aciduric streptococci, L. rhamnosus, and the lactic acid consuming organism V. dispar (FIG. 6). The pH of the biofilms at days 7, 9 and 11 was 6.20, 4.12 and 4.45, respectively. The overall numbers of organisms in the biofilms were reduced, due to likely growth inhibition from the acid conditions. These results demonstrated that urea played an essential role in maintaining community stability.

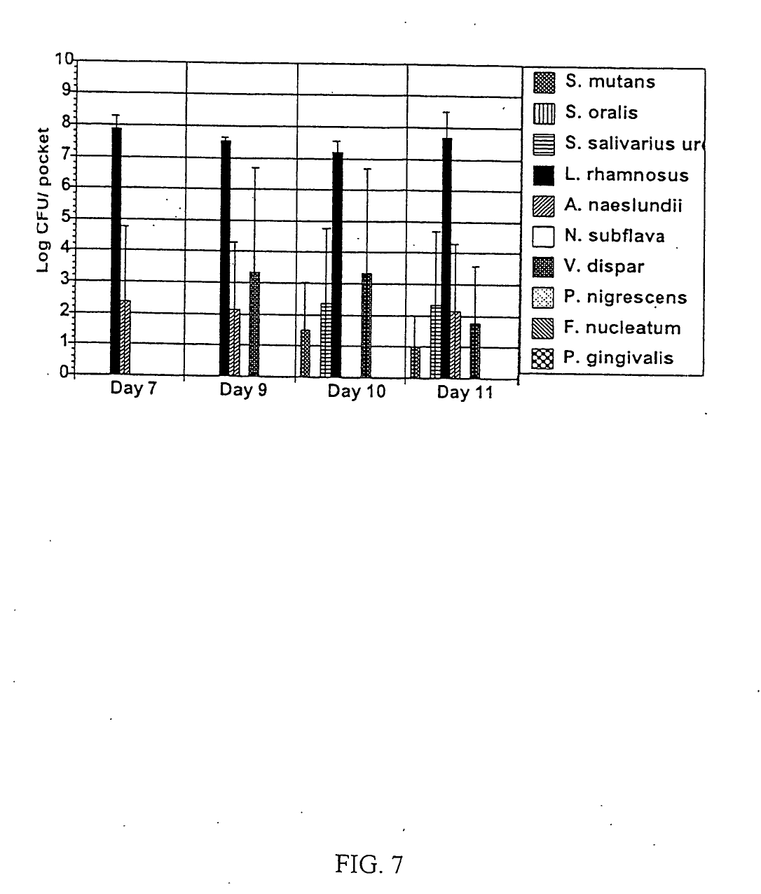

The importance of high levels of urease enzymes. To determine the role of the contribution of the individual urease enzymes of S. salivarius and A. naeslundii to maintaining the community stability, the 10-species consortium was constituted with nine of the wild-type organisms and a urease-deficient S. salivarius that was constructed by allelic exchange of the ureC gene (large subunit) of urease with a polar antibiotic resistance determinant (Chen and Byrne, 2000). Results of this study are shown in FIG. 7. In stark contrast to the previous results, in the absence of ureolytic S. salivarius, the 7-day biofilms were dominated primarily by lactobacilli and ureolytic A. naeslundii. Other organisms were present in numbers too low to detect. After cultivation for an additional 2, 3 or 4 days in the absence of urea, the biofilms comprised the aciduric streptococci and lactobacilli, the lactate-metabolizing Veillonella, and the urease-positive A. naeslundii. The pH of the biofilms at days 7, 9 and 11 was 4.12, 4.38 and 4.31, respectively. When biofilms were formed with urease-deficient strains of both S. salivarius and of A. naeslundii, the results were similar to those obtained with omission of the ureolytic S. salivarius.

These results clearly demonstrated that ammonia production by S. salivarius is an essential determinant for the establishment and persistence of diverse biofilms, preventing dominance of the biofilms by aciduric species. The presence of the urease-producing A. naeslundii was not sufficient to offset the impact of the loss of S. salivarius. The ineffectiveness of A. naeslundii in this regard may be explained by the fact that A. naeslundii is known to produce about 50-fold less urease than S. salivarius. The low level of urease produced by A. naeslundii may be such that it cannot provide sufficient ammonia from urea to avoid loss of community diversity.

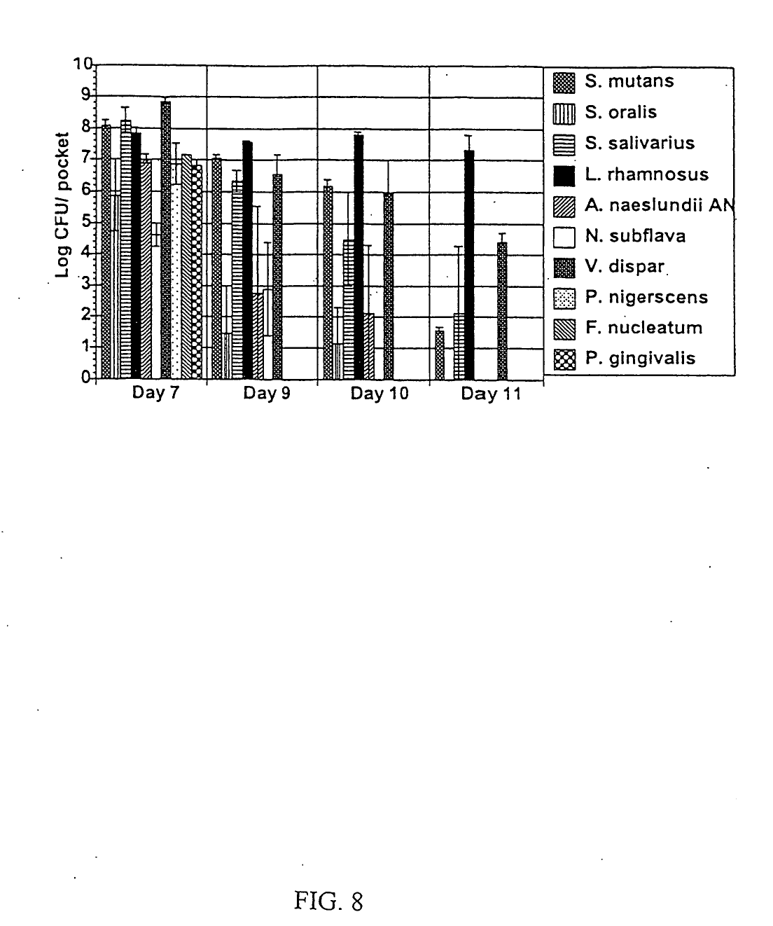

In a converse study, when a wild-type S. salivarius and urease-deficient A. naeslundii were utilized, community diversity after 7 days in BMMUG was essentially identical to that of biofilms formed with 10 wild-type species (FIG. 8), indicating that the amount of ammonia generated by the S. salivarius urease was adequate to create an environment conducive to the persistence of all species. However, removal of urea from the medium resulted in a rapid loss of diversity, similar to what was observed with the wild-type 10 species consortium (FIG. 6), confirming that urea was required for the effect on the communities. The pH of the biofilms at day 7 was 6.6, which was higher than when the wild-type A. naeslundii was included with the ureolytic S. salivarius (pH=6.1-6.2). This difference may indicate that there is competition for urea between the two organisms and that A. naeslundii incorporates more of the amino nitrogen of urea into proteins than is released to alkalinize the environment.

### Example 8

### Urease Activities in Dental Plague and Saliva of Caries-Active and Caries-Free Human Subjects

Materials and Methods:

Human subjects. To determine whether the levels of ammonia-generating capacity are lower in caries-active subjects than in caries-free individuals, twenty-five caries-free subjects (mean age=24.9 years old) and 8 caries-active subjects (mean age=26.6 years old) were recruited. Caries-active subjects had at least 6 open caries lesions, whereas caries-free subjects were individuals who had no evidence of caries experience in their permanent dentition. Informed consent was obtained. All of the subjects had good periodontal health, and were in good general health. None of the subjects was taking antibiotics during the study period.

Plaque and saliva collection. The participants refrained from tooth brushing for approximately 16 hours and had fasted overnight before sample collection. Supra-gingival plaque samples were collected from all tooth surfaces with the exception of the lingual surfaces of the lower anterior teeth of one half of an individual mouth as described (Tanaka and Margolis, 1999). Plaque from a single individual was pooled in a microcentrifuge tube comprising 0.5 ml of 10 mM sodium phosphate buffer (pH 8.0). The tube was kept on ice during sample collection. Approximately 2 ml of unstimulated saliva was collected from each participant by asking the subjects to expectorate into a chilled sterile plastic tube (Falcon 2070, Becton Dickinson and Company, Franklin Lakes, N.J.). The plaque and saliva samples were transferred to the laboratory for analysis within 15 minutes. The same procedure was carried out one week later for the other half of the mouth.

Urease assays. Urease activity was determined by measuring the amount of ammonia released by urea hydrolysis by fresh plaque and saliva samples. A 10 μl aliquot of each of the suspended plaque and saliva samples was incubated with 50 mM urea at 37° C. for 120 minutes in 50 mM potassium phosphate buffer, pH 7.0. The amount of ammonia released was measured using the Nessler Reagent (Aldrich Chemical Company, Inc, Milwaukee, Wis.) as previously described (Chen et al., 1996). Ammonium sulfate was used to prepare standard curves.

During the incubation period for the urease assays, suspended plaque and saliva samples were vortexed for 30 seconds. One portion (50 μl) was serially diluted in 0.5% proteose peptone and 0.25% KCl and plated onto Columbia blood agar (CBA, Columbia blood agar base, Difco, Md., plus 5% sheep blood). The plates were incubated at 37° C. in an anaerobic chamber (Plas Labs, Inc., Lansing, Mich.) for 7 days before counting colonies. The remainder of the suspended plaque and saliva samples were stored at −20° C.

Protein determination. When all samples were collected, total protein in the samples was measured as follows. The plaque samples were thawed on ice, 25011 of the sample was mixed with 250 μl of H₂O, and 500 μl of glass beads (0.1 mm, BioSpec Products, Inc., Bartlesville, Okla.) were added. The samples were then homogenized in a Bead Beater for 30 seconds, two times, with cooling on ice during the interval. For saliva samples, 500 μl of saliva was mixed with 500 μl of glass beads and homogenized as above. The samples were centrifuged for 5 minutes at 14,000 RPM in a refrigerated microcentrifuge. The protein concentration of the supernatant fluid was determined using the BioRad Protein assay reagent (BioRad Laboratories, Hercules, Calif.) with bovine serum albumin as the standard.

Statistical analyses. In an ANOVA analysis of the data, subjects were treated as random factors in a mixed model approach. Where appropriate, as in the case of urease activity normalized to plaque protein or CFU, a log transformation of the data was performed prior to the analysis.

Results:

The total protein and CFU in plaque and saliva of caries free and caries active subjects are presented in Table 2.

TABLE 2

| | Caries free | | Caries active | | |
|---|---|---|---|---|---|
| | (CF) | | (CA) | | |
| | n= 25 | | n= 8 | | |
| | Mean | | Mean | | |
| | (SE) | Range | (SE) | Range | p values |
| Plaque protein | 188.5 | 17.8- | 756.8 | 209.32- | 0.0001 |
| g/half mouth | (41.0) | 777.41 | (75.27) | 1376.36 | |
| Plaque CFU 10⁷ | 24.1 | 1.13- | 198.1 | 12.00- | 0.002 |
| in half mouth | (24.5) | 113 | (46.3) | 1460.00 | |
| Saliva protein | 1.15 | 0.45- | 1.23 | 0.56- | 0.616 |
| mg/ml | (0.08) | 1.92 | (0.14) | 3.21 | |
| Saliva CFU 10⁷/ml | 91.1 | 3.8- | 102.6 | 2.00- | 0.798 |
| | (20.82) | 810 | (38.86) | 414.00 | |

In general, the amount of plaque recoverable from the teeth of the caries-active subjects was significantly greater than that recovered from caries-free subjects. The total plaque protein in caries-active subjects averaged 4 times that found in the caries free subjects (p=0.0001). Similarly, the plaque CFU in caries-active subjects was roughly 10-fold higher than that of caries-free subjects (p=0.002).

There was no difference (p=0.616) in salivary protein concentrations between the caries active (1.23 mg/ml) and caries free subjects (1.15 mg/ml). The CFU in saliva of the caries-active subjects was slightly higher than that of the caries-free subjects, although the difference was not statistically significant (p=0.798).

Urease specific activities in the plaque and saliva of caries-free and caries-active subjects are presented in Table 3.

TABLE 3

| | Caries free (CF) | | Caries active (CA) | | |
|---|---|---|---|---|---|
| | n = 25 | | n = 8 | | |
| | Mean | | Mean | | |
| | (SE) | Range | (SE) | Range | p values |
| Plaque μmole urea | 3.11 | 0.19- | 1.0 | 0.09 | 0.0002* |
| hydrolyzed per | (0.49) | 22.62 | (0.89) | 2.67 | |
| mg of protein | | | | | |
| Plaque μmole urea | 3.64 | 0.43- | 1.72 | 0.044- | 0.0009* |
| per CFU10⁷ | (0.54) | 22.35 | (0.97) | 7.625 | |
| Saliva μmole urea | 0.41 | 0.048- | 0.58 | 0.032- | 0.222 |
| hydrolyzed per | (0.07) | 1.13 | (0.13) | 2.74 | |
| mg of protein | | | | | |
| Saliva mole urea | 53.20 | 10.88- | 81.17 | 9.9- | 0.222 |
| hydrolyzed per ml | (10.82) | 150 | (19.68) | 425 | |

*Log transformation was used in the data analysis.

Regardless of whether the data were normalized to protein or to CFU, plaque of caries-free subjects had significantly greater ureolytic capacity than that of caries-active subjects. Caries-free subjects harbored 3.1 Units of urease per milligram of protein, compared with 1.0 Units per milligram of protein for the caries-active subjects (p=0.0002). Similarly, caries-free subjects harbored 3.64 Units per 10⁷ CFU, as compared to less than half that value (1.72) for the caries-active subjects (p=0.0009).

Salivary urease activity in the caries-active subjects appeared somewhat higher than that of the caries free subjects, but this difference was not statistically significant, regardless of whether the data were normalized to protein or CFU (p=0.222).

The data show that there is an inverse relationship between the specific activity of urease in dental plaque and caries experience. However, total or normalized salivary urease activity did not differ significantly in these subjects, which implies that the relationship of ureolytic activity with caries in the two populations of subjects is not due to enhanced competition for the substrate between organisms in the plaque and the saliva. Results of this study provide compelling evidence that a loss of alkali-generating capacity in dental plaque is associated with the development and progression of caries.

### Example 9

### Production of Recombinant Bacteria Expressing AgDS

The general strategy for construction of plaque streptococci engineered to express genes of the agmatine deiminase system (AgDS) is essentially similar to that disclosed in Examples 2 and 5 above, which describe the construction of recombinant strains expressing, respectively, urease enzymes or genes of the arginine deiminase system. For construction of AgDS positive plaque streptococcal strains, standard cloning protocols are used to introduce into the host cells an agmatinine deiminase gene cluster, for example aguBDAC from S. mutans UA159 (SEQ ID NO:4), driven by the appropriate native or heterologous promoters. Bacterial strains, and vectors appropriate to the selected strains, are selected as described above.

### Example 10

### Description of Primers

SEQ ID NOS: 5-10

PCR Primers used for completing Streptococcus salivarius 57.I ure operon

1. Product #1:

(SEQ ID NO:5)

| | |
|---|---|
| S4420: | GCTTATCGGCTTGGTAGAAG |

(SEQ ID NO:6)

| | |
|---|---|
| TherAS17220: | GCTTCGTGCTTCAATCC |
| 2. Product #2: | |

(SEQ ID NO:7)

| | |
|---|---|
| 55290S: | GTTCTGCTCCAGTTAGATATCAC |

(SEQ ID NO:8)

| | |
|---|---|
| TherAS17750: | GCAATCTCATCATAGACAG |

3. Product #3

(SEQ ID NO:9)

| | |
|---|---|
| TherS17630: | CTCAAAGAATGGCTGAAGG |

(SEQ ID NO:10)

| | |
|---|---|
| TherAS18180: | ACCTAGAACGATTTCCTACT |

SEQ ID NOS: 11, 12

PCR Primers used to amplify complete structural gene encoding the α subunit of S. salivarius 57.I urease

(SEQ ID NO:11)

ureCBglIIS: 5′-TAGAAAGAGGACAGATCTATGAGTT-3′
and

(SEQ ID NO:12)

ureCSalIAS: 3′-CTCATTTATATAGGTCGACCCTTAGA.

SEQ ID NOS: 13-18

RT-PCR primer pairs used for detecting mRNA within S. salivarius 57.I ure operon:

1. between ureD and ureM:

(SEQ ID NO:13)

| | |
|---|---|
| S3660: | CTGAATTTAGAGTCTGATTTTGC |

(SEQ ID NO:14)

| | |
|---|---|
| AS4175: | GGCATTCGCACCAAAGGC |

2. between ureM and ureQ:

(SEQ ID NO:15)

| | |
|---|---|
| S4420: | GCTTATCGGCTTGGTAGAAG |

(SEQ ID NO:16)

| | |
|---|---|
| AS5045: | GGCTACAATCCACAAAACTGTG |

3. between ureQ and ureO:

(SEQ ID NO:17)

| | |
|---|---|
| S5290: | GTTCTGCTCCAGTTAGATATCAC |

(SEQ ID NO:18)

| | |
|---|---|
| AS17750: | GCAATCTCATCATAGACAG |

SEQ ID NOS: 19, 20

Degenerate PCR primers, designed based on alignments of known anabolic and catabolic OTCs, for generating an internal fragment of S. gordonii DL1 arcB, encoding cOTC

Primer 5′arcBS,
(5′-GGNGA(T/C)GCN(A/C)GNAA(T/C)AA(T/C)AT-3′;
SEQ ID NO:19),
Primer 3′arcBAS,
(5′-TGCATNC(G/T)(A/G)TT(T/C)TCNGC(T/G)TG-3′;
SEQ ID NO:20)

SEQ ID NO:21

Primer for primer extension analysis containing the antisense sequence of arcA located 42-bases 3′ to the translational start site.

AsarcA
(5′-CAGGTCTATGTAACATAACTTTTTTCA-3′; SEQ ID NO:21)

SEQ ID NO:22

Putative σ⁷⁰ promoter of arc operon of S. gordonii

(ParcA: TTGTGT-N₁₉-TAGAAT; SEQ ID NO:22)

SEQ ID NOS:23, 24

Regulatory sequenes (cre sites) 5′ to the transcription initiation site in arc operon of S. gordonii

AGAAAACGCTTCAA; SEQ ID NO:23,
spanning from −107 to −94
TGTAAGTGTTTTCA; SEQ ID NO:24,
spanning from −35 to −22

SEQ ID NOS:25, 26

Degenerate PCR primers, designed based on alignments of known anabolic and catabolic OTCs, for generating an internal fragment of S. rattus FA-1 arcB gene encoding cOTC.

(SEQ ID NO:25)

| | |
|---|---|
| Primer arcBS, | 5′-CAAGTATTTCAGGGACGC-3′, |

(SEQ ID NO:26)

| | |
|---|---|
| Primer arcBAS, | 5′-CATCTGTCAAGCCATTCC-3′ |

SEQ ID NO:27

Primer ArcAS
(5′-GACGATGTAACATTACCTTCTT-3′; SEQ ID NO:27)

(encodes the antisense sequence of S. rattus FA-1arcA located 50 bases downstream from the translational start site)
SEQ ID NOS:28-33

PCR primer pairs flanking the intergenic region of arcAB, arcDT and arcTR, respectively of S. rattus FA-1.

arcABS
(5′-CTGTGTATGTCTATGCCATTTG-3′; SEQ ID NO:28);
arcABAS
(5′-AGCTAGGAAACTGCGTCCCT-3′; SEQ ID NO:29);
arcDTS
(5′-TTTAGACTCTTTACAGGACAGATT-3′; SEQ ID NO:30);
arcDTAS
(5′-TGAATATTCATCTGTTTACCCCTT-3′; SEQ ID NO:31);
arcTRS
(5′-AGTGAGTTGTCTGAGTTTCTA-3′; SEQ ID NO:32);
and
arcTRAS
(5′-TTTATCTTACTTTGGCGCAATA-3′; SEQ ID NO:33).

SEQ ID NOS:34-37

Recombinant PCR primers for amplifying the S. rattus FA-1 arcA promoter and deletion derivatives, for ligation into 5′ end of promoterless chloramphenicol acetyltransferase gene (cat) from Staphylococcus aureus.

Sense primers:
arcASacI-S400
(TTGCTCTAGAGCTCTCAAATGACAGAA; SEQ ID NO:34);
arcASacI-S150
(TTATAAATTCGAGCTCCAAAAAACGTGAA; SEQ ID NO:35);
arcASacI-S100
(TAAATAACAATTCGAGCTCGAAAAAAATCTTA; SEQ ID NO:36)
Antisense primers:
arcABamHI-AS
(TTTTGAGTCATGGATCCTACTCCTTTCGAT; SEQ ID NO:37).

SEQ ID NOS:38, 39

PCR primer pair for amplification of the first strand aguB cDNA from S. mutans UA159

aguBS
(5′-CAGATTATATCTAGACAGAGGATTT-3′;
SEQ ID NO:38)
aguBAS
(5′-TACCAGCTGGGAATCCTTCTATCATTGTA-3′;
SEQ ID NO:39).

SEQ ID NOS:40-43

Primer pairs used in recombinant PCR to construct a polar aguB mutant in S. mutans. The first half of aguB was amplified using primer pairs:

aguBSXbaI
(5′-CAGATTATATCTAGACAGAGGATTT-3′;
SEQ ID NO:40)
and
aguBASEcoRI
(5′-TACCAGCTGGGAATTCTTCTATCATTGTA-3′;
SEQ ID NO:41),
(used for amplifying the first half of aguB)
aguBSEcoRI
(5′-TACAATGATAGAAGAATTCCCAGCTGGTA-3′;
SEQ ID NO:42)
and
aguBASSstI
(5′-ACCGTCCATGAGCTCATCTGTAATCT-3′;
SEQ ID NO:43)
(used for amplifing the remaining portion of aguB)

SEQ ID NOS:44, 45

PCR primer pair for generating an aguB-specific probe i.e., an internal fragment of S. mutans UA159 aguB, encoding a putative putrescine carbamoyltransferase:

aguBS
(5′-CAGATTATATCTAGACAGAGGATTT-3′;
SEQ ID NO:44)
aguBAS
(5′-TACCAGCTGGGAATCCTTCTATCATTGTA-3′;
SEQ ID NO:45).

SEQ ID NOS:46-50

Agmatine deiminase operon from S. mutans; amino acid sequences

(SEQ ID NO:46)

SMU.261c LuxR-like Transcriptional Regulator

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### CITED DOCUMENTS

The following literature citations as well as those cited above are incorporated in pertinent part by reference herein for the reasons cited in the above text:
Archibald, R. M., 1944. Determination of citrulline and allantoin and onstration of citrulline in blood plasma. J Biol Chem 156:121-142.
Ausubel, F. M., R. Brent, R. E. Kingston, D. D. Moore, J. G. Seidman, J. A. Smith, and K. Struhl, 1989. Current protocols in molecular biology. J. Wiley and Sons, New York, N.Y.
Bartolome, B., Y. Jubete, E. Martinez and F. de la Cruz, 1991. Construction and properties of a family of PACY184-derived cloning vectors compatible with pBR222. Gene 102:75-78.
Birnboim, H. C. and J. Doly, 1976. A rapid alkali extraction procedure for screening recombinant plasmid DNA. Nucleic Acids Res. 7:1513-1523.
Bradford, M. M., 1976. A rapid and sensitive method for the quantitation of microgram quantities of protein utilizing the principle of protein-dye binding. Anal. Biochem. 72:248-254.
Bradshaw, D. J., P. D. Marsh, K. M. Schilling, and D. Cummins, 1996. A modified chemostat system to study the ecology of oral biofilms. Journal of Appl Bacteriol 80:124-130.
Bradshaw, D. J., A. S. McKee, and P. D. Marsh, 1989. Effects of carbohydrate pulses and pH on population shifts within oral microbial communities in vitro. J Dent Res 68:1298-1302.
Burne, R. A., Z. T. Wen, Y.-Y. M Chen, and J. Penders, 1999. Regulation of expression of the fructan hydrolase of Streptococcus mutans GS-5 by induction and carbon catabolite repression. J. Bacteriol. 181:2863-2871.
Chen, Y. M., M. J. Betzenhauser and R. A. Burne, 2002. cis-acting elements that regulate the low-pH-inducible urease operon of Streptococcus salivarius. Microbiology 148:3599-3608.
Chen, Y. M., and R. A. Burne, 1996. Analysis of Streptococcus salivarius urease expression using continuous chemostat culture. FEMS Microbiol. Lett. 135:223-229.
Chen, Y. M., K. A. Clancy, and R. A. Burne, 1996. Streptococcus salivarius urease: genetic and biochemical characterization and expression in a dental plaque streptococcus. Infect. Immun. 64:585-592.
Chen, Y. M., C. A. Weaver, D. R. Mendelsohn and R. A. Burne, 1998. Transcriptional regulation of the Streptococcus salivarius 57.I urease operon. J. Bacteriol. 180:5769-5775.
Chen, Y. M., C. A. Weaver, and R. A. Burne, 2000. Dual functions of Streptococcus salivarius urease. J Bacteriol 182:4667-4669.
Cserzo, M., E. Wallin, I. Simon, G. von Heijne and A. Elofsson 1997. Prediction of the transmembrane α-helices in prokaryotic membrane proteins: the dense alignment surface method. Protein Eng. 10:673-676.
Curran, T. M., Y. Ma, G. C. Rutherford and R. E. Marquis, 1998. Turning on and off the arginine deiminase system in oral streptococci. Can. J, Microbiol. 44:1078-1085.
Higuchi, R., 1990. Recombinant PCR, p. 177-183. In M. A. Innis, D. H. Gelfand, and J. J. White (ed.), PCR protocols: a guide to methods and applications. Academic Press, Inc., CA.
Houghton, J. E., D. A. Bencini, G. A. O'Donovan, and J. R. Wild, 1984. Protein differentiation: a comparison of aspartate transcarbamylase and ornithine transcarbamylase from Escherichia coli K-12. Proc Natl Acad Sci USA 81:4864-8.
Grandori, R., T. A. Lavoie, M. Pflumm, G. Tian, H. Niersbach, W. K. Maas, R. Fairman and J. Carey, 1995. The DNA-binding domain of the hexameric arginine repressor. J. Mol. Biol. 254:150-162.
Kehres, D. G., C. H. Lawyer, and M. E. Maguire, 1998. The CorA magnesium transporter gene family. Microb. Comp. Genomics 3:151-169.
Kinniment, S. L., J. W. Wimpenny, D. Adams, and P. D. Marsh, 1996. Development of a steady-state oral microbial biofilm community using the constant-depth film fermenter. Microbiol. 142:631-638.
Knodler, L. A., E. O. Sekyere, T. S. Stewart, P. J. Schofield, and M. R. Edwards, 1998. Cloning and expression of a prokaryotic enzyme, arginine deiminase, from a primitive eukaryote Giardia intestinalis. J Biol Chem 273:4470-7.
LeBlanc, D. J., and F. P. Hassell, 1976. Transformation of Streptococcus sanguis strain Challis by plasmid DNA from Streptococcus faecalis. Journal of Bacteriology 128:347-355.
Lu, C.D., H. Winteler, A. Abdelal and D. Hass, 1999. The ArgR regulatory protein, a helper to the anaerobic regulator ANR during transcriptional activation of the arcD promoter in Pseudomonas aeruginosa. J. Bacteriol. 1810:6468-6475.
Maghnouj, A., T. F. de Sousa Cabra, V. Stalon and C. Vander Wauven, 1998. The arcABCD gene cluster encoding the arginine deiminase pathway of Bacillus licheniformis, and its activation by the arginine repressor ArgR. J. Bacteriol. 180:6468-6475.
Maas, W. K., 1994. The arginine repressor of Escherichia coli. Microbiol. Rev 58:631-40.
Mobley, H. L. T., M. D. Island, and R. P. Hausinger, 1995. Molecular biology of ureases. Microbiol. Rev. 59:451-480.
Morou-Bermudez, E., and R. A. Burne, 1999. Genetic and physiologic characterization of urease of Actinomyces naeslundii. Infect. Immun. 67:504-512.
Perez-Casal, J., M. G. Caparon, and J. R. Scott, 1991. Mry, a trans-acting positive regulator of the M protein gene of Streptococcus pyogenes with similarity to the receptor proteins of two-component regulatory systems. J. Bacteriol. 173:2617-2624.
Saier, M. H. J., S. Chauvaux, G. M. Cook, J. Deutscher, I. T. Paulsen, J. Reizer, and J.-J. Ye, 1996. Catabolite repression and induced control in Gram-positive bacteria. Microbiology 142:217-230.
Shaw, W. V., 1979. Chloramphenicol acetyltransferase activity from chloramphenicol-resistant bacteria. Methods in Enzymology 43:737-755.
Simon, J. P., and V. Stalon, 1982. Enzymes of agmatine degradation and the control of their synthesis in Streptococcus faecalis. J Bacteriol 152:676-81.
Smith, R. L., and M. E. Maguire, 1998. Microbial magnesium transport: unusual transporters searching for identity. Mol. Microbiol. 28:217-226.
Stulke, J. and W. Hillen, 1999. Carbon catabolite repression in bacteria. Curr. Opin. Microbiol. 2:195-201.
Tanaka M., Margolis H. C., 1999. Release of mineral ions in dental plaque following acid production. Arch. Oral Biol. 44:253-258.
Thompson, J. D., D. G. Higgins, and T. J. Gibson, 1994. CLUSTAL W: improving the sensitivity of progressive multiple sequence alignment through sequence weighting, position-specific gap penalties and weight matrix choice. Nucleic Acids Res 22:4673-80.
Tian, G., and W. K. Maas, 1994. Mutational analysis of the arginine repressor of Escherichia coli. Mol Microbiol 13:599-608.
Van Vliet, A. H., E. J. Kuipers, B. Waidner, B. J. Davies, N. de Vries, C. W. Penn, C. M. Vandenbroucke-Grauls, M. Kist, S. Bereswill, and J. G. Kusters, 2001. Nickel-responsive induction of urease expression in Helicobacter pylori is mediated at the transcriptional level. Infect Immun 69:4891-4897.
Wen, Z. T., and R. A. Burne, 2001. Construction of a new integration vector for use in Streptococcus mutans. Plasmid 45:31-36.
Wimpenny, J. W., 1997. The validity of models. Adv Dent Res 11:150-159.
Wolfram, L., B. Friedrich, and T. Eitinger. 1995. The Alcaligenes eutrophus protein HoxN mediates nickel transport in Escherichia coli. J. Bacteriol. 177:1840-1843.
Zuniga, M., M. Champomier-Verges, M. Zagorec and G. Perez-Martinez, 1998. Structural and functional analysis of the gene cluster encoding the enzymes of the arginine deiminase pathway of Lactobacillus sake. J. Bacteriol. 180:4154-4159.

What is claimed is:

1. A recombinant bacterial cell comprising an isolated nucleic acid construct, said cell expressing at least one alkalinizing enzyme.

2. The cell of claim 1, wherein said cell belongs to a bacterial strain that colonizes dental plaque.

3. The cell of claim 2, wherein said bacterial strain is at least one selected from the group consisting of Streptococcus mutans, Streptococcus sanguinis, Streptococcus gordonii, Streptococcus parasanguis, Streptococcus vestibularis, Streptococcus oralis, and Streptococcus mitis.

4. The cell of claim 1, wherein said alkalinizing enzyme is an ammonia-producing enzyme.

5. The cell of claim 4, wherein said ammonia-producing enzyme is a urease.

6. The cell of claim 4, wherein said ammonia-producing enzyme is an arginine deiminase.

7. The cell of claim 4, wherein said ammonia-producing enzyme is an agmatine deiminase.

8. The cell of claim 1, wherein said construct comprises at least one gene cluster encoding a urease and a nickel transporter.

9. The cell of claim 8, wherein said construct comprises ureIABCEFGDMQO.

10. The cell of claim 1, wherein said construct comprises at least one gene cluster encoding an arginine deiminase system.

11. The cell of claim 10, wherein said construct comprises arcABCDTR.

12. The cell of claim 1, wherein said construct comprises at least one gene cluster encoding an agmatine deiminase system.

13. The cell of claim 12, wherein said construct comprises aguBDAC and a transcriptional regulator located upstream of the agu gene cluster.

14. A method for reducing acidification of dental plaque in a subject, comprising the steps of:
providing a subject having at least one tooth with dental plaque disposed thereon, said tooth in a fluid environment, said fluid environment at a first pH;
contacting said tooth with a composition comprising at least one alkalinizing recombinant bacterial cell, said cell colonizing said dental plaque and producing at least one alkali from chemicals in said fluid environment, wherein said alkali raises a pH of said fluid environment to a second pH, said second pH higher than said first pH.

15. The method of claim 14, wherein said production of an alkali occurs in the absence of exogenous nickel.

16. The method of claim 14, wherein said alkali includes ammonia.

17. The method of claim 14, wherein said subject is a human.

18. A composition comprising at least one recombinant bacterial cell including an isolated nucleic acid construct, said cell expressing at least one alkalinizing enzyme, in a carrier.

19. The composition of claim 18, wherein said nucleic acid construct comprises at least one gene cluster encoding a urease enzyme.

20. The composition of claim 19, wherein said nucleic acid construct further comprises a nickel transporter.

21. The composition of claim 18, wherein said nucleic acid construct comprises at least one gene cluster encoding an arginine deiminase system.

22. The composition of claim 18, wherein said nucleic acid construct comprises at least one gene cluster encoding an agmatine deiminase system.

23. The composition of claim 18, wherein said carrier is selected from the group consisting of a chewing gum, a toothpaste, a lozenge, a powder, a gel, an ointment, a cream, a liquid, a mouthwash, a rinse, and a candy.

24. An isolated nucleic acid comprising the nucleotide sequence of SEQ ID NO: 1.

25. An isolated nucleic acid comprising the nucleotide sequence of SEQ ID NO:2.

26. An isolated nucleic acid comprising the nucleotide sequence of SEQ ID NO:3.

27. An isolated nucleic acid comprising the nucleotide sequence of SEQ ID NO:4.

28. The cell of claim 1, wherein said nucleic acid construct comprises a pMJB8 vector.

29. The cell of claim 1, wherein said nucleic acid construct comprises a pMC321 vector.

30. The cell of claim 1, wherein said nucleic construct comprises a pMC340A vector.

31. The cell of claim 1, wherein said nucleic construct comprises a pMC340B vector.

32. The cell of claim 1, wherein said nucleic construct comprises a pMC341A vector.

33. The cell of claim 1, wherein said nucleic construct comprises a pMC341B vector.

34. A composition comprising:
a host cell;
an insertion vector comprising nucleic acid sequences encoding an alkali producing enzyme and a nickel transporter; wherein,
the vector is stably integrated into the host cell genome.

35. The composition of claim 34, wherein the alkali producing enzyme is urease.

36. The composition of claim 34, wherein the host cell is selected from cells present in the oral cavity of a subject suffering from dental caries.

37. The composition of claim 34, wherein the host cell is selected from the genus Streptococcus.

38. The composition of claim 34, wherein the vector targets a mtl gene.

39. The composition of claim 38, wherein the vector further comprises nucleic acid sequences complementary to the mtl gene.

40. The composition of claim 38, wherein the vector invades the mtl gene molecule in the region of a target sequence by denaturing the bonds between the complementary target sequences of the double stranded molecule.

41. The composition of claim 38, wherein the vector is inserted into the mtl gene by double-crossover recombination.

42. The composition of claim 38, wherein the vector is integrated into the mtl gene in a single copy.

43. The composition of claim 38, wherein the host cell is selected by its inability to grow on mannitol.

44. The composition of claim 34, wherein the vector further comprises an entire urease gene cluster.

45. The composition of claim 44, wherein transcription of the urease gene cluster is under control of a strong promoter.

46. The composition of claim 44, wherein transcription of the urease gene cluster is under control of a tetracycline resistant promoter isolated from a tetracycline resistant gene.

47. The composition of claim 44, wherein transcription of the urease gene cluster is under control of a urease cognate promoter.

48. The composition of claim 34, wherein the host cell further comprises a detectable gene marker.

49. The composition of claim 48, wherein the detectable gene marker is an antibiotic resistance marker.

50. The composition of claim 34, wherein the host cell expresses urease enzymes in the absence of any exogenous nickel.

51. The composition of claim 50, wherein the host cell produces an extracellular alkali base.

52. The composition of claim 34, wherein the host cell secretes ammonia.

53. A method for treating dental caries comprising:
a recombinant cell expressing an alkali producing enzyme and a nickel transporter;
administering the recombinant host cell to an oral cavity of a subject suffering from or susceptible to dental caries; thereby,
neutralizing acidic pH in the subject and treating dental caries.

54. The method of claim 53, the alkali produced by the alkali producing enzyme is ammonia.

55. The method of claim 53, wherein the recombinant cell is administered in a dose from about 1×10⁴ up to 2×10⁸ per mg of bacterial weight.

56. The method of claim 53, wherein the recombinant cell produces an alkali, thereby raising acidic pH up to pH 8.

57. The method of claim 53, wherein the recombinant cell is belongs to a bacterial strain that colonizes dental plaque.

58. The method of claim 57, wherein the bacterial strain is at least one cell selected from the group consisting of Streptococcus mutans, Streptococcus sanguinis, Streptococcus gordonii, Streptococcus parasanguis, Streptococcus vestibularis, Streptococcus oralis, and Streptococcus mitis.

59. The method of claim 53, wherein the recombinant cell produces an alkali in the absence of exogenous nickel.
